(19) United States
(12) Patent Application Publication
(10) Pub. No.: US 2025/0289813 A1
(43) Pub. Date: Sep. 18, 2025

(54) CDK INHIBITORS, PHARMACEUTICAL COMPOSITIONS, AND THERAPEUTIC APPLICATIONS

(71) Applicant: ONQUALITY PHARMACEUTICALS CHINA LTD., Shanghai (CN)

(72) Inventors: Shilan LIU, Shanghai (CN); Shiyi ZHANG, Shanghai (CN); Chengxu ZHAO, Shanghai (CN)

(73) Assignee: ONQUALITY PHARMACEUTICALS CHINA LTD., Shanghai (CN)

(21) Appl. No.: 18/860,264

(22) PCT Filed: Apr. 24, 2023
(86) PCT No.: PCT/CN2023/090193

§ 371 Date: Oct. 25, 2024

(30) Foreign Application Priority Data
Apr. 25, 2022 (WO) PCT/CN2022/089004

(51) Int. Cl. C07D 471/04; A61K 31/519; A61K 31/5377; A61P 1/10; A61P 1/12

(52) U.S. Cl. CPC C07D 471/04; A61K 31/519; A61K 31/5377; A61P 1/10; A61P 1/12

(57) ABSTRACT

Provided herein are CDK inhibitors, e.g., a compound of Formula (I), and pharmaceutical compositions thereof. Also provide herein is a method of their use for treating, preventing, or ameliorating a chemotherapy-induced gastrointestinal side effect.

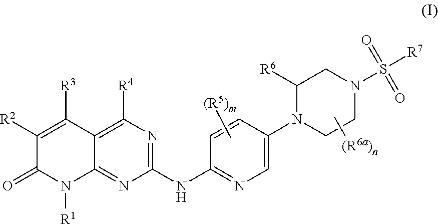

### CROSS REFERENCE TO RELATED APPLICATION

This application claims the benefit of the priority of International Application No. PCT/CN2022/089004, filed Apr. 25, 2022, under 35 U.S.C. 119(a); the disclosure of which is incorporated herein by reference in its entirety.

### FIELD

Provided herein are CDK inhibitors and pharmaceutical compositions thereof. Also provide herein is a method of their use for treating, preventing, or ameliorating a chemotherapy-induced gastrointestinal side effect.

### BACKGROUND

Chemotherapy-induced gastrointestinal side effects are common for cancer treatment and significantly affect morbidity and mortality of cancer patients. O'Reilly et al., Ther. Adv. Chronic Dis. 2020, 11, 2040622320970354; McQuade et al., Front Pharmacol. 2016, 7, 414. For example, the incidence of chemotherapy-induced diarrhea (CID) has been reported as high as 50-80% for conventional chemotherapy, such as fluorouracil and irinotecan. Stein, Ther. Adv. Med. Oncol. 2010, 2, 51-63; McQuade et al., Front Pharmacol. 2016, 7, 414. Surprisedly, many targeted therapies, such as tyrosine kinase inhibitors and immunotherapies, were found to have significantly higher risks of developing CIP than the conventional chemotherapy. Pessi et al., Crit. Rev. Oncol. Hematol. 2014, 90, 165-79; Bossi et al., Ann. Oncol. 2018, 29, iv126-42. CID has been observed in patients treated with small molecule tyrosine kinase inhibitors, vascular endothelial growth factor receptor (VEGFR) inhibitors, epidermal growth factor receptor (EGFR) inhibitors, multi-targeted TKIs, mammalian target of rapamycin (mTOR) inhibitors, cyclin-dependent kinase (CDK) 4/6 inhibitors, and poly(adenosine diphosphate-ribose) polymerase (PARP) inhibitors. Pessi et al., Crit. Rev. Oncol. Hematol. 2014, 90, 165-79; Bossi et al., Ann. Oncol. 2018, 29, iv126-42; Secombe et al., Integr. Cancer Ther. 2020, 19, 1-12.

Chemotherapy-induced gastrointestinal side effects often cause interruptions or changes to therapeutic regime and subsequently affect patient prognosis and overall survival. O'Reilly et al., Ther. Adv. Chronic Dis. 2020, 11, 2040622320970354; McQuade et al., Front Pharmacol. 2016, 7, 414. It has been reported that CID causes treatment alterations in approximately 60% of colorectal cancer patients, including dose reductions, and delays and cessation of treatment. Arbuckle et al., Oncologist 2000, 5, 250-9; Dranitsaris et al., Can. J. Gastroenterol. 2005, 19, 83-7. Current treatments for CID aim to reduce the severity of symptoms rather than combating the pathophysiological mechanisms of dysfunction. Andreyev et al., Lancet Oncol. 2014, 15, e447-e60; McQuade et al., Front Pharmacol. 2016, 7, 414. Therefore, there is a need for an effective therapy for treating, preventing, or ameliorating a chemotherapy-induced gastrointestinal side effect, such as CID.

### SUMMARY OF THE DISCLOSURE

Provided herein is a compound of Formula (I):

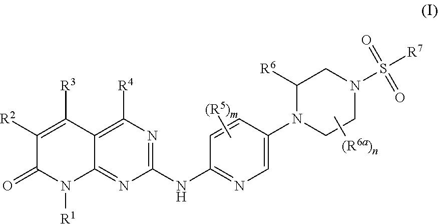

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein:
R¹ is (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, or —C(NR^{1a})NR^{1b}R^{1c};
R², R³, R⁴, and R⁶ are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R⁵ is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c};
R⁷ is (i) C₂₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —OR^{7a}, —NR^{7b}R^{7c}, —NR^{7d}C(O)R^{7e}, —NR^{7d}C(O)OR^{7e}, or —NR^{7d}C(O)NR^{7b}R^{7c};
each R^{6a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{7a} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{7b} and R^{7c} is independently hydrogen, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{7b} and R^{7c} together with the N atom to which they are attached form heteroaryl or heterocyclyl;
each R^{7d} and R^{7e} is independently hydrogen, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
m is an integer of 0, 1, 2, or 3; and
n is an integer of 0, 1, 2, 3, or 4;
wherein each alkyl, heteroalkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, nitrooxy, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, imino, nitro, nitrooxy, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{c})NR^{f}R^{g}, —C(S)R^{c}, —C(S)OR^{c}, —C(S)NR^{f}R^{g}, —OR^{c}, —OC(O)R^{c}, —OC(O)OR^{c}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{c}, —OS(O)₂R^{c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is a pharmaceutical composition comprising a compound of Formula (I), or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; and a pharmaceutically acceptable excipient.

Additionally, provided herein is a method of treating, preventing, or ameliorating one or more symptoms of a disorder, disease, or condition mediated by a cyclin dependent kinase (CDK) in a subject, comprising administering to the subject in need thereof a therapeutically effective amount of a compound of Formula (I), or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof.

Furthermore, provided herein is a method of treating, preventing, or ameliorating a chemotherapy-induced gastrointestinal side effect in a subject, comprising administering to the subject in need thereof a therapeutically effective amount of a compound of Formula (I), or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof.

Provided herein is a method of treating, preventing, or ameliorating a chemotherapy-induced diarrhea in a subject, comprising administering to the subject in need thereof a therapeutically effective amount of a compound of Formula (I), or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof.

Provided herein is a method of inhibiting the activity of a cyclin dependent kinase (CDK), comprising contacting the CDK with an effective amount of a compound of Formula (I), or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The term “heteroalkyl” refers to a linear or branched saturated monovalent hydrocarbon radical that contains one or more heteroatoms on its main chain, each independently selected from O, S, and N. The heteroalkyl is optionally substituted with one or more substituents Q as described herein. For example, C₁₋₆ heteroalkyl refers to a linear saturated monovalent hydrocarbon radical of 1 to 6 carbon atoms or a branched saturated monovalent hydrocarbon radical of 3 to 6 carbon atoms. In certain embodiments, the heteroalkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkyl groups are also referred as “lower heteroalkyl.” Examples of heteroalkyl groups include, but are not limited to, —OCH₃, —OCH₂CH₃, —CH₂OCH₃, —NHCH₃, —ONHCH₃, —NHOCH₃, —SCH₃, —CH₂NHCH₂CH₃, and —NHCH₂CH₂CH₃. Examples of substituted heteroalkyl groups include, but are not limited to, —CH₂NHC(O)CH₃ and —NHC(O)CH₂CH₃.

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The term “alkynyl” refers to a linear or branched monovalent hydrocarbon radical, which contains one or more, in one embodiment, one, two, three, or four, in another embodiment, one, carbon-carbon triple bond(s). An alkynyl group does not contain a carbon-carbon double bond. The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. For a heteroaryl group containing a heteroaromatic ring and a nonaromatic heterocyclic ring, the heteroaryl group is not bonded to the rest of a molecule through its nonaromatic heterocyclic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]pyridinyl, and imidazo[4,5-c]pyridinyl), imidazothiazolyl (including all isomeric forms, e.g., imidazo[2,1-b]thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]pyrimidinyl and [1,2,3]thiadiazolo[4,5-d]pyrimidinyl), and thienopyridyl (including all isomeric forms, e.g., thieno[2,3-b]pyridinyl, thieno[2,3-c]pyridinyl, thieno[3,2-b]pyridinyl, and thieno[3,2-c]pyridinyl). In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. For a heterocyclyl group containing a heteroaromatic ring and a nonaromatic heterocyclic ring, the heterocyclyl group is not bonded to the rest of a molecule through the heteroaromatic ring. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydro-benzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydro-pyranyl, dihydropyrazolyl, dihydropyrazinyl, dihydropyridinyl, dihydropyrimidinyl, dihydro-pyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “halogen,” “halide,” or “halo” refers to fluoro, chloro, bromo, and/or iodo.

The term “optionally substituted” is intended to mean that a group or substituent, such as an alkyl, heteroalkyl, alkenyl, alkynyl, cycloalkyl, aryl, aralkyl, heteroaryl, or heterocyclyl group, may be substituted with one or more, in one embodiment, one, two, three, or four, substituents Q, each of which is independently selected from, e.g., (a) deuterium (-D), cyano (—CN), halo, imino (═NH), nitro (—NO₂), and oxo (═O); (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)R^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{c}, —C(O)OR^{c}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{c}, —OC(NR^{c})NR^{f}R^{g}, —OC(S)R^{c}, —OC(S)OR^{c}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{c}C(O)R^{h}, —NR^{c}C(O)OR^{f}, —NR^{c}C(O)NR^{f}R^{g}, —NR^{c}C(O)SR^{f}, —NR^{c}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{c}S(O)₂NR^{f}R^{g}, —P(O)R^{f}R^{g}, —SR^{c}, —S(O)R^{c}, —S(O)₂R^{c}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

For a divalent group described herein, no orientation is implied by the direction in which the divalent group is presented. For example, unless a particular orientation is specified, the formula —C(O)NH— represents both —C(O)NH— and —NHC(O)—.

The phrase “an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof” has the same meaning as the phrase “(i) an enantiomer, a mixture of enantiomers, a diastereomer, a mixture of two or more diastereomers, a tautomer, or a mixture of two or more tautomers of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a diastereomer, a mixture of two or more diastereomers, a tautomer, or a mixture of two or more tautomers of the compound referenced therein.”

### Compounds

In one embodiment, provided herein is a compound of Formula (I):

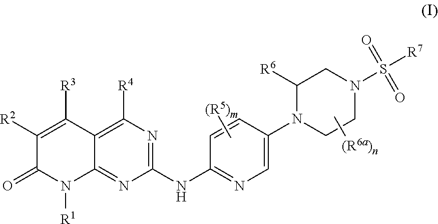

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein:
R¹ is (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, or —C(NR^{1a})NR^{1b}R^{1c};
R², R³, R⁴, and R⁶ are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R⁵ is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c};
R⁷ is (i) C₂₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —OR^{7a}, —NR^{7b}R^{7c}, —NR^{7d}C(O)R^{7c}, —NR^{7d}C(O)OR^{7e}, or —NR^{7d}C(O)NR^{7b}R^{7c};
each R^{6a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{7a} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{7b} and R^{7c} is independently hydrogen, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{7b} and R^{7c} together with the N atom to which they are attached form heteroaryl or heterocyclyl;
each R^{7d} and R^{7e} is independently hydrogen, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
m is an integer of 0, 1, 2, or 3; and
n is an integer of 0, 1, 2, 3, or 4;
wherein each alkyl, heteroalkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, nitrooxy, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, imino, nitro, nitrooxy, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{c}, —OS(O)₂R^{c}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{c}S(O)₂NR^{f}R^{g}, —SR^{c}, —S(O)R^{c}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In another embodiment, provided herein is a compound of Formula (Ia):

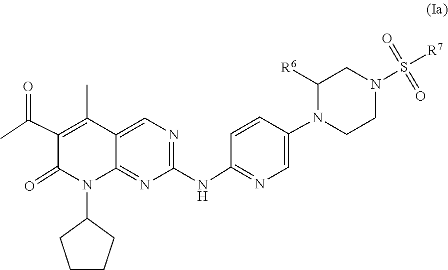

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶ and R⁷ are each as defined herein.

In yet another embodiment, provided herein is a compound of Formula (Ib):

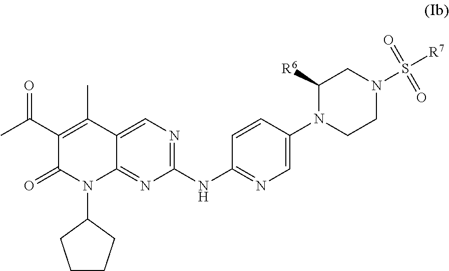

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶ and R⁷ are each as defined herein.

In certain embodiments, in Formula (I), (Ia), or (Ib), R⁶ is hydrogen or C₁₋₆ alkyl, optionally substituted with one or more substituents Q. In certain embodiments, in Formula (I), (Ia), or (Ib), R⁶ is hydrogen or methyl. In certain embodiments, in Formula (I), (Ia), or (Ib), R⁶ is hydrogen. In certain embodiments, in Formula (I), (Ia), or (Ib), R⁶ is methyl.

In certain embodiments, in Formula (I), (Ia), or (Ib), R⁷ is (i) C₂₋₆ alkyl, heteroaryl, or heterocyclyl, each optionally substituted with one or more substituents Q; or (ii) —OR^{7a}, —NR^{7b}R^{7c}, —NR^{7d}C(O)R^{7e}, —NR^{7d}C(O)OR^{7e}, or —NR^{7d}C(O)NR^{7b}R^{7c}, wherein each R^{7a}, R^{7b}, R^{7c}, R^{7d}, and R^{7e} is as defined herein. In certain embodiments, in Formula (I), (Ia), or (Ib), R⁷ is C₂₋₆ alkyl, optionally substituted with one or more substituents Q. In certain embodiments, in Formula (I), (Ia), or (Ib), R⁷ is ethyl, propyl, or butyl, each optionally substituted with one, two, or three substituents Q. In certain embodiments, in Formula (I), (Ia), or (Ib), R⁷ is ethyl or isopropyl.

In certain embodiments, in Formula (I), (Ia), or (Ib), R⁷ is heteroaryl, optionally substituted with one or more substituents Q. In certain embodiments, in Formula (I), (Ia), or (Ib), R⁷ is monocyclic heteroaryl, optionally substituted with one or more substituents Q. In certain embodiments, in Formula (I), (Ia), or (Ib), R⁷ is 5- or 6-membered heteroaryl, each optionally substituted with one or more substituents Q. In certain embodiments, in Formula (I), (Ia), or (Ib), R⁷ is 5-membered heteroaryl, optionally substituted with one, two, or three substituents Q. In certain embodiments, in Formula (I), (Ia), or (Ib), R⁷ is 6-membered heteroaryl, optionally substituted with one, two, or three substituents Q. In certain embodiments, in Formula (I), (Ia), or (Ib), R⁷ is imidazolyl, optionally substituted with one, two, or three substituents Q. In certain embodiments, in Formula (I), (Ia), or (Ib), R⁷ is imidazol-1-yl, optionally substituted with one, two, or three substituents Q. In certain embodiments, in Formula (I), (Ia), or (Ib), R⁷ is imidazol-1-yl or 2-aminoimidazol-1-yl.

In certain embodiments, in Formula (I), (Ia), or (Ib), R⁷ is bicyclic heteroaryl, optionally substituted with one or more substituents Q. In certain embodiments, in Formula (I), (Ia), or (Ib), R⁷ is 5,5-, 5,6-, or 6,6-fused heteroaryl, each optionally substituted with one or more substituents Q. In certain embodiments, in Formula (I), (Ia), or (Ib), R⁷ is 5,5-fused heteroaryl, optionally substituted with one, two, or three substituents Q. In certain embodiments, in Formula (I), (Ia), or (Ib), R⁷ is 5,6-fused heteroaryl, optionally substituted with one, two, or three substituents Q. In certain embodiments, in Formula (I), (Ia), or (Ib), R⁷ is 6,6-fused heteroaryl, optionally substituted with one, two, or three substituents Q.

In certain embodiments, in Formula (I), (Ia), or (Ib), R⁷ is heterocyclyl, optionally substituted with one or more substituents Q. In certain embodiments, in Formula (I), (Ia), or (Ib), R⁷ is monocyclic heterocyclyl, optionally substituted with one or more substituents Q. In certain embodiments, in Formula (I), (Ia), or (Ib), R⁷ is 3-, 4-, 5-, 6-, or 7-membered heterocyclyl, each optionally substituted with one or more substituents Q. In certain embodiments, in Formula (I), (Ia), or (Ib), R⁷ is 3-membered heterocyclyl, optionally substituted with one, two, or three substituents Q. In certain embodiments, in Formula (I), (Ia), or (Ib), R⁷ is 4-membered heterocyclyl, optionally substituted with one, two, or three substituents Q. In certain embodiments, in Formula (I), (Ia), or (Ib), R⁷ is 5-membered heterocyclyl, optionally substituted with one, two, or three substituents Q. In certain embodiments, in Formula (I), (Ia), or (Ib), R⁷ is 6-membered heterocyclyl, optionally substituted with one, two, or three substituents Q. In certain embodiments, in Formula (I), (Ia), or (Ib), R⁷ is 7-membered heterocyclyl, optionally substituted with one, two, or three substituents Q. In certain embodiments, in Formula (I), (Ia), or (Ib), R⁷ is azetidinyl, oxazolidinyl, piperidinyl, or morpholinyl, each optionally substituted with one, two, or three substituents Q. In certain embodiments, in Formula (I), (Ia), or (Ib), R⁷ is azetidin-1-yl, oxazolidin-3-yl, piperidin-1-yl, or morpholin-4-yl, each optionally substituted with one, two, or three substituents Q. In certain embodiments, in Formula (I), (Ia), or (Ib), R⁷ is 3-hydroxyazetidin-1-yl, 5-(hydroxymethyl)-2-oxooxazolidin-3-yl, (R)-2-(hydroxymethyl)piperidin-1-yl, or morpholin-4-yl.

In certain embodiments, in Formula (I), (Ia), or (Ib), R⁷ is bicyclic heterocyclyl, optionally substituted with one or more substituents Q. In certain embodiments, in Formula (I), (Ia), or (Ib), R⁷ is bridged, fused, or spiro heterocyclyl, each optionally substituted with one or more substituents Q. In certain embodiments, in Formula (I), (Ia), or (Ib), R⁷ is bridged heterocyclyl, optionally substituted with one, two, or three substituents Q. In certain embodiments, in Formula (I), (Ia), or (Ib), R⁷ is fused heterocyclyl, optionally substituted with one, two, or three substituents Q. In certain embodiments, in Formula (I), (Ia), or (Ib), R⁷ is spiro heterocyclyl, optionally substituted with one, two, or three substituents Q.

In certain embodiments, in Formula (I), (Ia), or (Ib), R⁷ is —OR^{7a}, wherein R^{7a} is as defined herein. In certain embodiments, in Formula (I), (Ia), or (Ib), R⁷ is —OR^{7a}, wherein R^{7a} is C₁₋₆ alkyl, C₃₋₁₀ cycloalkyl, or C₆₋₁₄ aryl, each optionally substituted with one or more substituents Q. In certain embodiments, in Formula (I), (Ia), or (Ib), R⁷ is C₁₋₆ alkoxy, optionally substituted with one, two, or three substituents Q. In certain embodiments, in Formula (I), (Ia), or (Ib), R⁷ is methoxy, optionally substituted with one, two, or three substituents Q. In certain embodiments, in Formula (I), (Ia), or (Ib), R⁷ is C₃₋₁₀ cycloalkoxy, optionally substituted with one, two, or three substituents Q. In certain embodiments, in Formula (I), (Ia), or (Ib), R⁷ is C₆₋₁₄ aryloxy, optionally substituted with one, two, or three substituents Q. In certain embodiments, in Formula (I), (Ia), or (Ib), R⁷ is methoxy, ethoxy, propoxy, cyclopentoxy, or phenoxy, each optionally substituted with one, two, or three substituents Q. In certain embodiments, in Formula (I), (Ia), or (Ib), R⁷ is pivaloyloxymethoxy, benzoyloxymethoxy, isopropoxy, cyclopentoxy, or 4-methylphenoxy.

In certain embodiments, in Formula (I), (Ia), or (Ib), R⁷ is —NR^{7b}R^{7c}, wherein R^{7b} and R^{7c} are each as defined herein. In certain embodiments, in Formula (I), (Ia), or (Ib), R⁷ is —NR^{7b}R^{7c}, wherein R^{7b} and R^{7c} are each independently hydrogen or C₁₋₆ alkyl, optionally substituted with one or more substituents Q. In certain embodiments, in Formula (I), (Ia), or (Ib), R⁷ is —NR^{7b}R^{7c}, wherein R^{7b} and R^{7c} are each independently hydrogen or C₁₋₆ alkyl, optionally substituted with hydroxycarbonyl, hydroxyl, methoxy, or amino. In certain embodiments, in Formula (I), (Ia), or (Ib), R⁷ is —NR^{7b}R^{7c}, wherein R^{7b} and R^{7c} are each independently (i) hydrogen; or (ii) methyl, ethyl, propyl, butyl, or pentyl, each optionally substituted with hydroxycarbonyl, hydroxyl, methoxy, or amino. In certain embodiments, in Formula (I), (Ia), or (Ib), R⁷ is —NR^{7b}R^{7c}, wherein R^{7b} and R^{7c} are each independently hydrogen, methyl, hydroxycarbonylmethyl, ethyl, 2-hydroxyethyl, 2-methoxyethyl, propyl, isopropyl, or 1-(hydroxycarbonyl)-2-methylpropyl.

In certain embodiments, in Formula (I), (Ia), or (Ib), R⁷ is —NR^{7b}R^{7c}, wherein R^{7b} and R^{7c} together with the N atom to which they are attached form heteroaryl or heterocyclyl, each optionally substituted with one or more substituents Q. In certain embodiments, in Formula (I), (Ia), or (Ib), R⁷ is —NR^{7b}R^{7c}, wherein R^{7b} and R^{7c} together with the N atom to which they are attached form heteroaryl, optionally substituted with one or more substituents Q. In certain embodiments, in Formula (I), (Ia), or (Ib), R⁷ is —NR^{7b}R^{7c}, wherein R^{7b} and R^{7c} together with the N atom to which they are attached form monocyclic heteroaryl, optionally substituted with one, two, or three substituents Q. In certain embodiments, in Formula (I), (Ia), or (Ib), R⁷ is —NR^{7b}R^{7c}, wherein R^{7b} and R^{7c} together with the N atom to which they are attached form 5- or 6-heteroaryl, each optionally substituted with one, two, or three substituents Q. In certain embodiments, in Formula (I), (Ia), or (Ib), R⁷ is —NR^{7b}R^{7c}, wherein R^{7b} and R^{7c} together with the N atom to which they are attached form 5-heteroaryl, optionally substituted with one, two, or three substituents Q. In certain embodiments, in Formula (I), (Ia), or (Ib), R⁷ is —NR^{7b}R^{7c}, wherein R^{7b} and R^{7c} together with the N atom to which they are attached form bicyclic heteroaryl, optionally substituted with one, two, or three substituents Q.

In certain embodiments, in Formula (I), (Ia), or (Ib), R⁷ is —NR^{7b}R^{7c}, wherein R^{7b} and R^{7c} together with the N atom to which they are attached form heterocyclyl, optionally substituted with one or more substituents Q. In certain embodiments, in Formula (I), (Ia), or (Ib), R⁷ is —NR^{7b}R^{7c}, wherein R^{7b} and R^{7c} together with the N atom to which they are attached form monocyclic heterocyclyl, optionally substituted with one, two, or three substituents Q. In certain embodiments, in Formula (I), (Ia), or (Ib), R⁷ is —NR^{7b}R^{7c}, wherein R^{7b} and R^{7c} together with the N atom to which they are attached form 3-, 4-, 5-, 6-, or 7-membered heterocyclyl, each optionally substituted with one, two, or three substituents Q. In certain embodiments, in Formula (I), (Ia), or (Ib), R⁷ is —NR^{7b}R^{7c}, wherein R^{7b} and R^{7c} together with the N atom to which they are attached form 5- or 6-membered heterocyclyl, each optionally substituted with one, two, or three substituents Q. In certain embodiments, in Formula (I), (Ia), or (Ib), R⁷ is —NR^{7b}R^{7c}, wherein R^{7b} and R^{7c} together with the N atom to which they are attached form 5-membered heterocyclyl, optionally substituted with one, two, or three substituents Q. In certain embodiments, in Formula (I), (Ia), or (Ib), R⁷ is —NR^{7b}R^{7c}, wherein R^{7b} and R^{7c} together with the N atom to which they are attached form 6-membered heterocyclyl, optionally substituted with one, two, or three substituents Q. In certain embodiments, in Formula (I), (Ia), or (Ib), R⁷ is —NR^{7b}R^{7c}, wherein R^{7b} and R^{7c} together with the N atom to which they are attached form bicyclic heterocyclyl, optionally substituted with one, two, or three substituents Q.

In certain embodiments, in Formula (I), (Ia), or (Ib), R⁷ is —NR^{7b}R^{7c}, wherein R^{7b} and R^{7c} together with the N atom to which they are attached form imidazol-1-yl, 2-amino-imidazol-1-yl, 3-hydroxyazetidin-1-yl, 2-oxo-5-(hydroxymethyl)oxazolidin-3-yl, 2-(hydroxy-methyl)piperidin-1-yl, or morpholin-4-yl.

In certain embodiments, in Formula (I), (Ia), or (Ib), R⁷ is —NR^{7d}C(O)R^{7e}, wherein R^{7d} and R^{7e} are each as defined herein. In certain embodiments, in Formula (I), (Ia), or (Ib), R⁷ is —NR^{7d}C(O)R^{7e}, wherein R^{7d} is hydrogen; and wherein R^{7e} is C₁₋₆ alkyl, C₃₋₁₀ cycloalkyl, or heterocyclyl, each optionally substituted with one or more substituents Q. In certain embodiments, in Formula (I), (Ia), or (Ib), R⁷ is —NR^{7d}C(O)R^{7e}, wherein R^{7d} is hydrogen; and wherein R^{7e} is C₁₋₆ alkyl, optionally substituted with one, two, or three substituents Q. In certain embodiments, in Formula (I), (Ia), or (Ib), R⁷ is —NR^{7d}C(O)R^{7e}, wherein R^{7d} is hydrogen; and wherein R^{7c} is C₃₋₁₀ cycloalkyl, optionally substituted with one, two, or three substituents Q. In certain embodiments, in Formula (I), (Ia), or (Ib), R⁷ is —NR^{7d}C(O)R^{7e}, wherein R^{7d} is hydrogen; and wherein R^{7e} is monocyclic C₃₋₁₀ cycloalkyl, optionally substituted with one, two, or three substituents Q. In certain embodiments, in Formula (I), (Ia), or (Ib), R⁷ is —NR^{7d}C(O)R^{7e}, wherein R^{7d} is hydrogen; and wherein R^{7c} is heterocyclyl, optionally substituted with one, two, or three substituents Q. In certain embodiments, in Formula (I), (Ia), or (Ib), R⁷ is —NR^{7d}C(O)R^{7c}, wherein R^{7d} is hydrogen; and wherein R^{7c} is monocyclic heterocyclyl, optionally substituted with one, two, or three substituents Q. In certain embodiments, in Formula (I), (Ia), or (Ib), R⁷ is —NR^{7d}C(O)R^{7c}, wherein R^{7d} is hydrogen; and wherein R^{7c} is R^{7c} is 3-, 4-, 5-, 6-, or 7-membered heterocyclyl, each optionally substituted with one, two, or three substituents Q. In certain embodiments, in Formula (I), (Ia), or (Ib), R⁷ is —NR^{7d}C(O)R^{7c}, wherein R^{7d} is hydrogen; and wherein R^{7e} is R^{7e} is 5- or 6-membered heterocyclyl, each optionally substituted with one, two, or three substituents Q. In certain embodiments, in Formula (I), (Ia), or (Ib), R⁷ is —NR^{7d}C(O)R^{7e}, wherein R^{7d} is hydrogen; and wherein R^{7e} is methyl, ethyl, propyl, pentyl, cyclopropyl, cyclobutyl, pyrrolidinyl, tetrahydrofuryl, or tetrahydropyranyl, each optionally substituted with one, two, or three substituents Q. In certain embodiments, in Formula (I), (Ia), or (Ib), R⁷ is —NR^{7d}C(O)R^{7c}, wherein R^{7d} is hydrogen; and wherein R^{7c} is methyl, aminomethyl, ethyl, 1-aminoethyl, 1-amino-2-(imidazol-4-yl)ethyl, propyl, isopropyl, 1-aminopropyl, 1-amino-2-methylpropyl, 1,5-diaminopentyl, cyclopropyl, cyclobutyl, pyrrolidin-2-yl, tetrahydrofuran-2-yl, or tetrahydropyran-4-yl. In certain embodiments, in Formula (I), (Ia), or (Ib), R⁷ is acetamido, 2-aminoacetamido, propanamido, 2-aminopropanamido, 2-amino-3-(imidazol-4-yl)propanamido, butanamido, isobutanamido, 2-aminobutanamido, 2-amino-3-methylbutan-amido, 2,6-diaminohexanamido, cyclopropylamido, cyclobutylamido, pyrrolidin-2-ylamido, tetrahydrofuran-2-carboxamido, or tetrahydropyran-4-carboxamido. In certain embodiments, in Formula (I), (Ia), or (Ib), R⁷ is acetamido, 2-aminoacetamido, propanamido, (S)-2-aminopropanamido, (S)-2-amino-3-(imidazol-4-yl)propanamido, butanamido, isobutanamido, (S)-2-aminobutanamido, 2-amino-3-methylbutanamido, (S)-2,6-diaminohexanamido, cyclopropylamido, cyclobutylamido, (S)-pyrrolidin-2-ylamido, tetrahydrofuran-2-carboxamido, or tetrahydropyran-4-carboxamido.

In certain embodiments, in Formula (I), (Ia), or (Ib), R⁷ is —NR^{7d}C(O)OR^{7e}, wherein R^{7d} and R^{7e} are each as defined herein. In certain embodiments, in Formula (I), (Ia), or (Ib), R⁷ is —NR^{7d}C(O)OR^{7e}, wherein R^{7d} is hydrogen; and wherein R^{7e} is C₁₋₆ alkyl, C₃₋₁₀ cycloalkyl, or heterocyclyl, each optionally substituted with one or more substituents Q. In certain embodiments, in Formula (I), (Ia), or (Ib), R⁷ is —NR^{7d}C(O)OR^{7e}, wherein R^{7d} is hydrogen; and wherein R^{7e} is C₁₋₆ alkyl, optionally substituted with one, two, or three substituents Q. In certain embodiments, in Formula (I), (Ia), or (Ib), R⁷ is —NR^{7d}C(O)OR^{7e}, wherein R^{7d} is hydrogen; and wherein R^{7e} is C₃₋₁₀ cycloalkyl, optionally substituted with one, two, or three substituents Q. In certain embodiments, in Formula (I), (Ia), or (Ib), R⁷ is —NR^{7d}C(O)OR^{7e}, wherein R^{7d} is hydrogen; and wherein R^{7e} is monocyclic C₃₋₁₀ cycloalkyl, optionally substituted with one, two, or three substituents Q. In certain embodiments, in Formula (I), (Ia), or (Ib), R⁷ is —NR^{7d}C(O)OR^{7e}, wherein R^{7d} is hydrogen; and wherein R^{7e} is heterocyclyl, optionally substituted with one, two, or three substituents Q. In certain embodiments, in Formula (I), (Ia), or (Ib), R⁷ is —NR^{7d}C(O)OR^{7e}, wherein R^{7d} is hydrogen; and wherein R^{7e} is monocyclic heterocyclyl, optionally substituted with one, two, or three substituents Q. In certain embodiments, in Formula (I), (Ia), or (Ib), R⁷ is —NR^{7d}C(O)OR^{7e}, wherein R^{7d} is hydrogen; and wherein R^{7c} is 3-, 4-, 5-, 6-, or 7-membered heterocyclyl, each optionally substituted with one, two, or three substituents Q. In certain embodiments, in Formula (I), (Ia), or (Ib), R⁷ is —NR^{7d}C(O)OR^{7e}, wherein R^{7d} is hydrogen; and wherein R^{7e} is 5- or 6-membered heterocyclyl, each optionally substituted with one, two, or three substituents Q.

In certain embodiments, in Formula (I), (Ia), or (Ib), R⁷ is —NR^{7d}C(O)OR^{7e}, wherein R^{7d} is hydrogen; and wherein R^{7e} is methyl, hydroxycarbonylmethyl, ethoxycarbonylmethyl, ethyl, 2-hydroxyethyl, 2-methoxyethyl, 2-acetoxyethyl, 2-aminoethyl, 2-acetamidoethyl, 2-(2-aminoacetamido)ethyl, 2-(2-aminopropanamido)ethyl, 2-(2-amino-3-hydroxypropanamido)-ethyl, (2-(2-amino-3-methylbutanamido)ethyl, 2-(2-amino-4-(methylthio)butanamido)ethyl, 2-(2-amino-4-methylpentanamido)ethyl, 2-(pyrrolidin-2-ylamido)ethyl, isopropyl, tert-butyl, cyclopropyl, cyclobutyl, or tetrahydropyran-4-yl. In certain embodiments, in Formula (I), (Ia), or (Ib), R⁷ is —NR^{7d}C(O)OR^{7e}, wherein R^{7d} is hydrogen; and wherein R^{7e} is methyl, hydroxycarbonylmethyl, ethoxycarbonylmethyl, ethyl, 2-hydroxyethyl, 2-methoxyethyl, 2-acetoxyethyl, 2-aminoethyl, 2-acetamidoethyl, 2-(2-aminoacetamido)ethyl, (R)-2-(2-aminopropanamido)ethyl, (S)-2-(2-amino-3-hydroxypropanamido)ethyl, (S)-2-(2-amino-3-methylbutanamido)ethyl, (S)-2-(2-amino-4-(methylthio)butanamido)ethyl, (S)-2-(2-amino-4-methylpentanamido)ethyl, (S)-2-(pyrrolidin-2-ylamido)ethyl, isopropyl, tert-butyl, cyclopropyl, cyclobutyl, or tetrahydropyran-4-yl.

In certain embodiments, in Formula (I), (Ia), or (Ib), R⁷ is methoxycarbonylamino, hydroxycarbonylmethoxycarbonylamino, ethoxycarbonylmethoxycarbonylamino, ethoxycarbonylamino, 2-hydroxyethoxycarbonylamino, 2-methoxyethoxycarbonylamino, 2-acetoxyethoxycarbonylamino, 2-aminoethoxycarbonylamino, 2-acetamidoethoxycarbonylamino, 2-(2-aminoacetamido)ethoxycarbonylamino, 2-(2-aminopropanamido)ethoxycarbonylamino, 2-(2-amino-3-hydroxypropanamido)ethoxycarbonylamino, 2-(2-amino-3-methylbutan-amido)ethoxycarbonylamino, 2-(2-amino-4-(methylthio)butanamido)ethoxycarbonylamino, 2-(2-amino-4-methylpentanamido)-ethoxycarbonylamino, 2-(pyrrolidin-2-ylamido)ethoxycarbonylamino, isopropoxycarbonylamino, tert-butoxycarbonylamino, cyclopropoxycarbonylamino, cyclobutoxycarbonylamino, or tetrahydropyran-4-oxycarbonylamino. In certain embodiments, in Formula (I), (Ia), or (Ib), R⁷ is methoxycarbonylamino, hydroxycarbonylmethoxycarbonylamino, ethoxycarbonylmethoxycarbonylamino, ethoxycarbonylamino, 2-hydroxyethoxycarbonylamino, 2-methoxyethoxycarbonylamino, 2-acetoxyethoxycarbonylamino, 2-aminoethoxycarbonylamino, 2-acetamidoethoxycarbonylamino, 2-(2-aminoacetamido)ethoxycarbonylamino, (R)-2-(2-aminopropanamido)ethoxycarbonylamino, (S)-2-(2-amino-3-hydroxy-propanamido)ethoxycarbonylamino, (S)-2-(2-amino-3-methylbutanamido)ethoxycarbonylamino, (S)-2-(2-amino-4-(methylthio)butanamido)ethoxycarbonylamino, (S)-2-(2-amino-4-methyl-pentanamido)ethoxycarbonylamino, (S)-2-(pyrrolidin-2-ylamido)ethoxycarbonylamino, isopropoxycarbonylamino, tert-butoxycarbonylamino, cyclopropoxycarbonylamino, cyclobutoxycarbonylamino, or tetrahydropyran-4-oxycarbonylamino.

In certain embodiments, in Formula (I), (Ia), or (Ib), R⁷ is —NR^{7d}C(O)NR^{7b}R^{7c}, wherein R^{7b}, R^{7c}, and R^{7d} are each as defined herein. In certain embodiments, in Formula (I), (Ia), or (Ib), R⁷ is —NR^{7d}C(O)NR^{7b}R^{7c}, wherein R^{7b} and R^{7d} are each hydrogen; and wherein R^{7c} is C₁₋₆ alkyl, optionally substituted with one or more substituents Q. In certain embodiments, in Formula (I), (Ia), or (Ib), R⁷ is N-isopropylureido.

In certain embodiments, in Formula (I), (Ia), or (Ib), R⁷ is ethyl, isopropyl, imidazol-1-yl, 2-aminoimidazol-1-yl, 3-hydroxyazetidin-1-yl, 5-(hydroxymethyl)-2-oxo-oxazolidin-3-yl, (R)-2-(hydroxymethyl)piperidin-1-yl, morpholin-4-yl, pivaloyloxymethoxy, benzoyloxymethoxy, isopropoxy, cyclopentoxy, 4-methylphenoxy, amino, acetamido, 2-amino-acetamido, propanamido, (S)-2-aminopropanamido, (S)-2-amino-3-(imidazol-4-yl)propanamido, butanamido, isobutanamido, (S)-2-aminobutanamido, 2-amino-3-methylbutanamido, (S)-2,6-diaminohexanamido, cyclopropylamido, cyclobutylamido, (S)-pyrrolidin-2-ylamido, tetrahydro-furan-2-carboxamido, tetrahydropyran-4-carboxamido, methoxycarbonylamino, hydroxycarbonylmethoxycarbonylamino, ethoxycarbonylmethoxycarbonylamino, ethoxycarbonylamino, 2-hydroxyethoxycarbonylamino, 2-methoxyethoxycarbonylamino, 2-acetoxyethoxycarbonylamino, 2-aminoethoxycarbonylamino, 2-acetamidoethoxycarbonylamino, 2-(2-amino-acetamido)ethoxycarbonylamino, (R)-2-(2-aminopropanamido)ethoxycarbonylamino, (S)-2-(2-amino-3-hydroxypropanamido)ethoxycarbonylamino, (S)-2-(2-amino-3-methylbutanamido)-ethoxycarbonylamino, (S)-2-(2-amino-4-(methylthio)butanamido)ethoxycarbonylamino, (S)-2-(2-amino-4-methylpentanamido)ethoxycarbonylamino, (S)-2-(pyrrolidin-2-ylamido)ethoxycarbonylamino, isopropoxycarbonylamino, tert-butoxycarbonylamino, cyclopropoxycarbonylamino, cyclobutoxycarbonylamino, tetrahydropyran-4-oxycarbonylamino, or N-isopropylureido.

In yet another embodiment, provided herein is a compound of Formula (II):

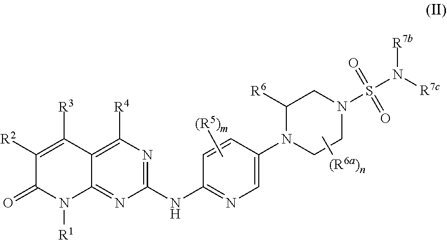

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R¹, R², R³, R⁴, R⁵, R⁶, R^{6a}, R^{7b}, R^{7c}, m, and n are each as defined herein.

In yet another embodiment, provided herein is a compound of Formula (IIa):

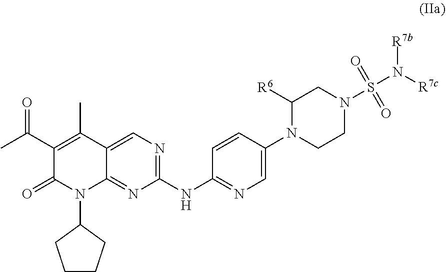

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶, R^{7b}, and R^{7c} are each as defined herein.

In yet another embodiment, provided herein is a compound of Formula (IIb):

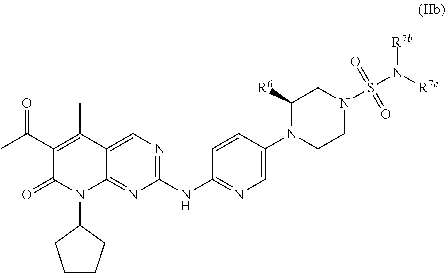

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶, R^{7b}, and R^{7c} are each as defined herein.

In certain embodiments, in Formula (II), (IIa), or (IIb), R⁶ is hydrogen or C₁₋₆ alkyl, optionally substituted with one or more substituents Q. In certain embodiments, in Formula (II), (IIa), or (IIb), R⁶ is hydrogen or methyl. In certain embodiments, in Formula (II), (IIa), or (IIb), R⁶ is hydrogen. In certain embodiments, in Formula (II), (IIa), or (IIb), R⁶ is methyl.

In certain embodiments, in Formula (II), (IIa), or (IIb), R^{7b} and R^{7c} are each as defined herein. In certain embodiments, in Formula (II), (IIa), or (IIb), R^{7b} and R^{7c} are each independently hydrogen or C₁₋₆ alkyl, optionally substituted with one or more substituents Q. In certain embodiments, in Formula (II), (IIa), or (IIb), R⁷ is —NR^{7b}R^{7c}, wherein R^{7b} and R^{7c} are each independently hydrogen or C₁₋₆ alkyl, optionally substituted with hydroxycarbonyl, hydroxyl, methoxy, or amino. In certain embodiments, in Formula (II), (IIa), or (IIb), R⁷ is —NR^{7b}R^{7c}, wherein R^{7b} and R^{7c} are each independently (i) hydrogen; or (ii) methyl, ethyl, propyl, butyl, or pentyl, each optionally substituted with hydroxycarbonyl, hydroxyl, methoxy, or amino. In certain embodiments, in Formula (II), (IIa), or (IIb), R^{7b} and R^{7c} are each independently hydrogen, methyl, hydroxycarbonylmethyl, ethyl, 2-hydroxyethyl, 2-methoxyethyl, propyl, isopropyl, or 1-(hydroxycarbonyl)-2-methylpropyl.

In certain embodiments, in Formula (II), (IIa), or (IIb), R^{7b} and R^{7c} together with the N atom to which they are attached form heteroaryl or heterocyclyl, each optionally substituted with one or more substituents Q. In certain embodiments, in Formula (II), (IIa), or (IIb), R^{7b} and R^{7c} together with the N atom to which they are attached form heteroaryl, optionally substituted with one or more substituents Q. In certain embodiments, in Formula (II), (IIa), or (IIb), R^{7b} and R^{7c} together with the N atom to which they are attached form monocyclic heteroaryl, optionally substituted with one, two, or three substituents Q. In certain embodiments, in Formula (II), (IIa), or (IIb), R^{7b} and R^{7c} together with the N atom to which they are attached form 5- or 6-membered heteroaryl, each optionally substituted with one, two, or three substituents Q. In certain embodiments, in Formula (II), (IIa), or (IIb), R^{7b} and R^{7c} together with the N atom to which they are attached form 5-membered heteroaryl, optionally substituted with one, two, or three substituents Q.

In certain embodiments, in Formula (II), (IIa), or (IIb), R^{7b} and R^{7c} together with the N atom to which they are attached form heterocyclyl, optionally substituted with one or more substituents Q. In certain embodiments, in Formula (II), (IIa), or (IIb), R^{7b} and R^{7c} together with the N atom to which they are attached form monocyclic heterocyclyl, optionally substituted with one, two, or three substituents Q. In certain embodiments, in Formula (II), (IIa), or (IIb), R^{7b} and R^{7c} together with the N atom to which they are attached form 3-, 4-, 5-, 6-, or 7-membered heterocyclyl, each optionally substituted with one, two, or three substituents Q. In certain embodiments, in Formula (II), (IIa), or (IIb), R^{7b} and R^{7c} together with the N atom to which they are attached form 5- or 6-membered heterocyclyl, each optionally substituted with one, two, or three substituents Q. In certain embodiments, in Formula (II), (IIa), or (IIb), R^{7b} and R^{7c} together with the N atom to which they are attached form 5-membered heterocyclyl, optionally substituted with one, two, or three substituents Q. In certain embodiments, in Formula (II), (IIa), or (IIb), R^{7b} and R^{7c} together with the N atom to which they are attached form 6-membered heterocyclyl, optionally substituted with one, two, or three substituents Q.

In certain embodiments, in Formula (II), (IIa), or (IIb), R^{7b} and R^{7c} together with the N atom to which they are attached form imidazol-1-yl, 2-aminoimidazol-1-yl, 3-hydroxyazetidin-1-yl, 2-oxo-5-(hydroxymethyl)oxazolidin-3-yl, 2-(hydroxymethyl)piperidin-1-yl, or morpholin-4-yl.

In yet another embodiment, provided herein is a compound of Formula (III):

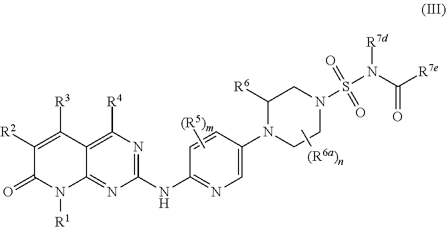

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R¹, R², R³, R⁴, R⁵, R⁶, R^{6a}, R^{7d}, R^{7e}, m, and n are each as defined herein.

In yet another embodiment, provided herein is a compound of Formula (IIIa):

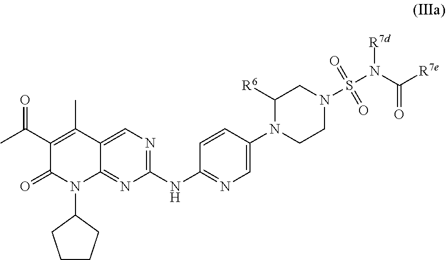

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶, R^{7d}, and R^{7c} are each as defined herein.

In yet another embodiment, provided herein is a compound of Formula (IIIb):

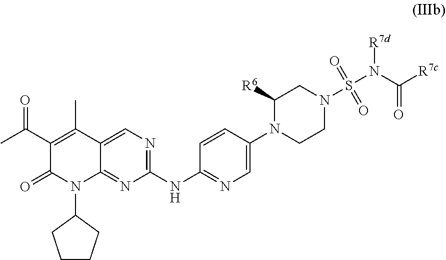

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶, R^{7d}, and R^{7c} are each as defined herein.

In certain embodiments, in Formula (III), (IIIa), or (IIIb), R⁶ is hydrogen or C₁₋₆ alkyl, optionally substituted with one or more substituents Q. In certain embodiments, in Formula (III), (IIa), or (IIIb), R⁶ is hydrogen or methyl. In certain embodiments, in Formula (III), (IIIa), or (IIIb), R⁶ is hydrogen. In certain embodiments, in Formula (III), (IIIa), or (IIIb), R⁶ is methyl.

In certain embodiments, in Formula (III), (IIIa), or (IIIb), R^{7d} is hydrogen; and R^{7e} is C₁₋₆ alkyl, C₃₋₁₀ cycloalkyl, or heterocyclyl, each optionally substituted with one or more substituents Q. In certain embodiments, in Formula (III), (IIIa), or (IIIb), R^{7d} is hydrogen; and R^{7e} is C₁₋₆ alkyl, optionally substituted with one, two, or three substituents Q. In certain embodiments, in Formula (II), (IIIa), or (IIIb), R^{7d} is hydrogen; and R^{7e} is C₃₋₁₀ cycloalkyl, optionally substituted with one, two, or three substituents Q. In certain embodiments, in Formula (III), (IIa), or (IIIb), R^{7d} is hydrogen; and R^{7c} is monocyclic C₃₋₁₀ cycloalkyl, optionally substituted with one, two, or three substituents Q. In certain embodiments, in Formula (III), (IIIa), or (IIIb), R^{7d} is hydrogen; and R^{7c} is heterocyclyl, optionally substituted with one, two, or three substituents Q. In certain embodiments, in Formula (III), (IIIa), or (IIIb), R^{7d} is hydrogen; and R^{7e} is monocyclic heterocyclyl, optionally substituted with one, two, or three substituents Q. In certain embodiments, in Formula (III), (IIIa), or (IIIb), R^{7d} is hydrogen; and R^{7c} is 3-, 4-, 5-, 6-, or 7-membered heterocyclyl, each optionally substituted with one, two, or three substituents Q. In certain embodiments, in Formula (III), (IIIa), or (IIIb), R^{7d} is hydrogen; and R^{7c} is 5- or 6-membered heterocyclyl, each optionally substituted with one, two, or three substituents Q. In certain embodiments, in Formula (III), (IIIa), or (IIIb), R^{7d} is hydrogen; and R^{7c} is methyl, ethyl, propyl, pentyl, cyclopropyl, cyclobutyl, pyrrolidinyl, tetrahydrofuryl, or tetrahydropyranyl, each optionally substituted with one, two, or three substituents Q. In certain embodiments, in Formula (III), (IIIa), or (IIIb), R^{7d} is hydrogen; and R^{7e} is methyl, aminomethyl, ethyl, 1-aminoethyl, 1-amino-2-(imidazol-4-yl)ethyl, propyl, isopropyl, 1-aminopropyl, 1-amino-2-methylpropyl, 1,5-diaminopentyl, cyclopropyl, cyclobutyl, pyrrolidin-2-yl, tetrahydrofuran-2-yl, or tetrahydropyran-4-yl.

In yet another embodiment, provided herein is a compound of Formula (IV):

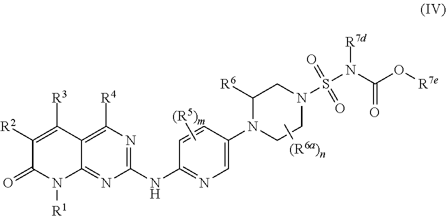

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R¹, R², R³, R⁴, R⁵, R⁶, R^{6a}, R^{7d}, R^{7e}, m, and n are each as defined herein.

In yet another embodiment, provided herein is a compound of Formula (IVa):

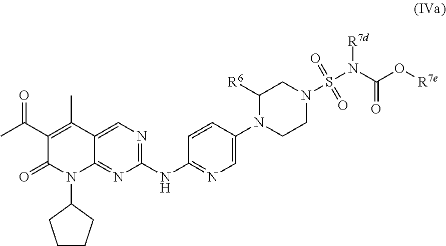

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof, wherein R⁶, R^{7d}, and R^{7e} are each as defined herein.

In yet another embodiment, provided herein is a compound of Formula (IVb):

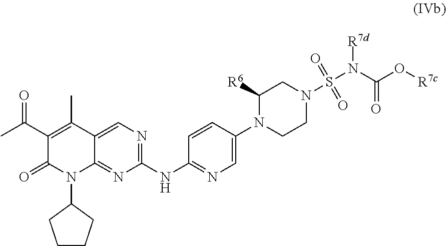

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶, R^{7d}, and R^{7e} are each as defined herein.

In certain embodiments, in Formula (IV), (IVa), or (IVb), R⁶ is hydrogen or C₁₋₆ alkyl, optionally substituted with one or more substituents Q. In certain embodiments, in Formula (IV), (IVa), or (IVb), R⁶ is hydrogen or methyl. In certain embodiments, in Formula (IV), (IVa), or (IVb), R⁶ is hydrogen. In certain embodiments, in Formula (IV), (IVa), or (IVb), R⁶ is methyl.

In certain embodiments, in Formula (IV), (IVa), or (IVb), R^{7d} is hydrogen; and R^{7e} is C₁₋₆ alkyl, C₃₋₁₀ cycloalkyl, or heterocyclyl, each optionally substituted with one or more substituents Q. In certain embodiments, in Formula (IV), (IVa), or (IVb), R^{7d} is hydrogen; and R^{7e} is C₁₋₆ alkyl, optionally substituted with one, two, or three substituents Q. In certain embodiments, in Formula (IV), (IVa), or (IVb), R^{7d} is hydrogen; and R^{7e} is C₃₋₁₀ cycloalkyl, optionally substituted with one, two, or three substituents Q. In certain embodiments, in Formula (IV), (IVa), or (IVb), R^{7d} is hydrogen; and R^{7e} is monocyclic C₃₋₁₀ cycloalkyl, optionally substituted with one, two, or three substituents Q. In certain embodiments, in Formula (IV), (IVa), or (IVb), R^{7d} is hydrogen; and R^{7e} is heterocyclyl, optionally substituted with one, two, or three substituents Q. In certain embodiments, in Formula (IV), (IVa), or (IVb), R^{7d} is hydrogen; and R^{7c} is monocyclic heterocyclyl, optionally substituted with one, two, or three substituents Q. In certain embodiments, in Formula (IV), (IVa), or (IVb), R^{7d} is hydrogen; and R^{7e} is 3-, 4-, 5-, 6-, or 7-membered heterocyclyl, each optionally substituted with one, two, or three substituents Q. In certain embodiments, in Formula (IV), (IVa), or (IVb), R^{7d} is hydrogen; and R^{e} is 5- or 6-membered heterocyclyl, each optionally substituted with one, two, or three substituents Q. In certain embodiments, in Formula (IV), (IVa), or (IVb), R^{7d} is hydrogen; and R^{7e} is 5-membered heterocyclyl, optionally substituted with one, two, or three substituents Q. In certain embodiments, in Formula (IV), (IVa), or (IVb), R^{7d} is hydrogen; and R^{7e} is 6-membered heterocyclyl, optionally substituted with one, two, or three substituents Q.

In certain embodiments, in Formula (IV), (IVa), or (IVb), R^{7d} is hydrogen; and R^{7c} is C₁₋₆ alkyl, optionally substituted with one, two, or three substituents, wherein each substituent is independently —C(O)OR^{1a}, —OR^{1a}, —OC(O)R^{1a}, —NR^{1b}R^{1c}, or —NR^{1a}C(O)R^{1d}; and wherein each R^{1a}, R^{1b}, R^{1c}, and R^{1d} is as defined herein. In certain embodiments, in Formula (IV), (IVa), or (IVb), R^{7d} is hydrogen; and R^{7e} is C₁₋₆ alkyl, substituted with —C(O)OR^{1a}, wherein R^{1a} is as defined herein. In certain embodiments, in Formula (IV), (IVa), or (IVb), R^{7d} is hydrogen; and R^{7e} is C₁₋₆ alkyl, substituted with —OR^{1a}, wherein R^{1a} is as defined herein. In certain embodiments, in Formula (IV), (IVa), or (IVb), R^{7d} is hydrogen; and R^{7e} is C₁₋₆ alkyl, substituted with —OC(O)R^{1a}, wherein R^{1a} is as defined herein. In certain embodiments, in Formula (IV), (IVa), or (IVb), R^{7d} is hydrogen; and R^{7e} is C₁₋₆ alkyl, substituted with —NR^{1b}R^{1e}, wherein R^{1b} and R^{1c} are each as defined herein. In certain embodiments, in Formula (IV), (IVa), or (IVb), R^{7d} is hydrogen; and R^{7c} is C₁₋₆ alkyl, substituted with —NR^{1a}C(O)R^{1d}, wherein R^{1a} and R^{1d} are each as defined herein.

In certain embodiments, in Formula (IV), (IVa), or (IVb), R^{7d} is hydrogen; and R^{7e} is C₁₋₆ alkyl substituted with aminoacylamino. In certain embodiments, in Formula (IV), (IVa), or (IVb), R^{7d} is hydrogen; and R^{7e} is C₁₋₆ alkyl substituted with α-aminoacylamino or (3-aminoacylamino. In certain embodiments, in Formula (IV), (IVa), or (IVb), R^{7d} is hydrogen; and R^{7e} is C₁₋₆ alkyl substituted with α-aminoacylamino, which is aminoacetamido, 2-aminopropanamido, 2-amino-3-phenylpropanamido, 2-amino-3-(4-hydroxyphenyl)propanamido, 2-amino-3-(imidazol-4-yl)propanamido, 2-amino-3-(indol-3-yl)propanamido, 2-amino-3-(hydroxy-carbonyl)propanamido, 2-amino-3-(aminocarbonyl)propanamido, 2-amino-3-hydroxypropanamido, 2-amino-3-mercaptopropanamido, 2-amino-3-methylbutanamido, 2-amino-3-hydroxybutanamido, 2-amino-4-(methylthio)butanamido, 2-amino-4-hydroxycarbonylbutan-amido, 2-amino-4-(aminocarbonyl)butanamido, 2-amino-3-methylpentanamido, 2-amino-4-methylpentanamido, 2,6-diaminohexanamido, 2-amino-5-guanidinopentamido, or pyrrolin-2-ylcarboxamido. In certain embodiments, in Formula (IV), (IVa), or (IVb), R^{7d} is hydrogen; and R^{7c} is C₁₋₆ alkyl substituted with α-aminoacylamino, which is aminoacetamido, (R)-2-aminopropanamido, (R)-2-amino-3-phenylpropanamido, (R)-2-amino-3-(4-hydroxyphenyl)propanamido, (R)-2-amino-3-(imidazol-4-yl)propanamido, (R)-2-amino-3-(indol-3-yl)propanamido, (R)-2-amino-3-(hydroxy-carbonyl)propanamido, (R)-2-amino-3-(aminocarbonyl)propanamido, (R)-2-amino-3-hydroxy-propanamido, (R)-2-amino-3-mercaptopropanamido, (R)-2-amino-3-methylbutanamido, (R)-2-amino-3-hydroxybutanamido, (R)-2-amino-4-(methylthio)butanamido, (R)-2-amino-4-hydroxycarbonylbutanamido, (R)-2-amino-4-(aminocarbonyl)butanamido, (R)-2-amino-3-methylpentanamido, (R)-2-amino-4-methylpentanamido, (R)-2,6-diaminohexanamido, (R)-2-amino-5-guanidinopentamido, or (R)-pyrrolin-2-ylcarboxamido. In certain embodiments, in Formula (IV), (IVa), or (IVb), R^{7d} is hydrogen; and R^{7c} is C₁₋₆ alkyl substituted with α-aminoacylamino, which is aminoacetamido, (S)-2-aminopropanamido, (S)-2-amino-3-phenylpropanamido, (S)-2-amino-3-(4-hydroxyphenyl)propanamido, (S)-2-amino-3-(imidazol-4-yl)propanamido, (S)-2-amino-3-(indol-3-yl)propanamido, (S)-2-amino-3-(hydroxycarbonyl)propanamido, (S)-2-amino-3-(aminocarbonyl)propanamido, (S)-2-amino-3-hydroxypropanamido, (S)-2-amino-3-mercaptopropanamido, (S)-2-amino-3-methylbutanamido, (S)-2-amino-3-hydroxybutanamido, (S)-2-amino-4-(methylthio)butanamido, (S)-2-amino-4-hydroxycarbonylbutanamido, (S)-2-amino-4-(aminocarbonyl)butanamido, (S)-2-amino-3-methylpentanamido, (S)-2-amino-4-methylpentanamido, (S)-2,6-diaminohexanamido, (S)-2-amino-5-guanidinopentamido, or (S)-pyrrolin-2-ylcarboxamido.

In certain embodiments, in Formula (IV), (IVa), or (IVb), R^{7d} is hydrogen; and R^{7e} is ethyl substituted with aminoacylamino. In certain embodiments, in Formula (IV), (IVa), or (IVb), R^{7d} is hydrogen; and R^{7e} is ethyl substituted at the 2-position with α-aminoacylamino or β-aminoacylamino. In certain embodiments, in Formula (IV), (IVa), or (IVb), R^{7d} is hydrogen; and R^{7c} is and R^{7c} is ethyl substituted at the 2-position with α-aminoacylamino, which is aminoacetamido, 2-aminopropanamido, 2-amino-3-phenylpropanamido, 2-amino-3-(4-hydroxy-phenyl)propanamido, 2-amino-3-(imidazol-4-yl)propanamido, 2-amino-3-(indol-3-yl)propanamido, 2-amino-3-(hydroxycarbonyl)propanamido, 2-amino-3-(aminocarbonyl)propanamido, 2-amino-3-hydroxypropanamido, 2-amino-3-mercaptopropanamido, 2-amino-3-methylbutan-amido, 2-amino-3-hydroxybutanamido, 2-amino-4-(methylthio)butanamido, 2-amino-4-hydroxycarbonylbutanamido, 2-amino-4-(aminocarbonyl)butanamido, 2-amino-3-methylpentanamido, 2-amino-4-methylpentanamido, 2,6-diaminohexanamido, 2-amino-5-guanidinopentamido, or pyrrolin-2-ylcarboxamido. In certain embodiments, in Formula (IV), (IVa), or (IVb), R^{7d} is hydrogen; and R^{7c} is ethyl substituted at the 2-position with α-aminoacylamino, which is aminoacetamido, (R)-2-aminopropanamido, (R)-2-amino-3-phenylpropanamido, (R)-2-amino-3-(4-hydroxyphenyl)propanamido, (R)-2-amino-3-(imidazol-4-yl)propanamido, (R)-2-amino-3-(indol-3-yl)propanamido, (R)-2-amino-3-(hydroxycarbonyl)propanamido, (R)-2-amino-3-(aminocarbonyl)propanamido, (R)-2-amino-3-hydroxypropanamido, (R)-2-amino-3-mercaptopropanamido, (R)-2-amino-3-methylbutanamido, (R)-2-amino-3-hydroxybutanamido, (R)-2-amino-4-(methylthio)butanamido, (R)-2-amino-4-hydroxycarbonyl-butanamido, (R)-2-amino-4-(aminocarbonyl)butanamido, (R)-2-amino-3-methylpentanamido, (R)-2-amino-4-methylpentanamido, (R)-2,6-diaminohexanamido, (R)-2-amino-5-guanidinopentamido, or (R)-pyrrolin-2-ylcarboxamido. In certain embodiments, in Formula (IV), (IVa), or (IVb), R^{7d} is hydrogen; and R^{7c} is ethyl substituted at the 2-position with α-aminoacylamino, which is aminoacetamido, (S)-2-aminopropanamido, (S)-2-amino-3-phenylpropanamido, (S)-2-amino-3-(4-hydroxyphenyl)-propanamido, (S)-2-amino-3-(imidazol-4-yl)propanamido, (S)-2-amino-3-(indol-3-yl)propanamido, (S)-2-amino-3-(hydroxycarbonyl)propanamido, (S)-2-amino-3-(aminocarbonyl)propanamido, (S)-2-amino-3-hydroxypropanamido, (S)-2-amino-3-mercaptopropanamido, (S)-2-amino-3-methylbutanamido, (S)-2-amino-3-hydroxybutanamido, (S)-2-amino-4-(methylthio)butanamido, (S)-2-amino-4-hydroxycarbonylbutanamido, (S)-2-amino-4-(aminocarbonyl)butanamido, (S)-2-amino-3-methylpentanamido, (S)-2-amino-4-methylpentanamido, (S)-2,6-diaminohexanamido, (S)-2-amino-5-guanidinopentamido, and (S)-pyrrolin-2-ylcarboxamido.

In certain embodiments, in Formula (IV), (IVa), or (IVb), R^{7d} is hydrogen; and R^{7e} is methyl, ethyl, propyl, or butyl, each optionally substituted with one, two, or three substituents, wherein each substituent is independently hydroxycarbonyl, ethoxycarbonyl, hydroxyl, methoxy, acetoxy, amino, acetamido, aminoacetamido, 2-aminopropanamido, 2-amino-3-hydroxy-propanamido, 2-amino-3-methylbutanamido, 2-amino-4-(methylthio)butanamido, 2-amino-4-methylpentanamido, or pyrrolidin-2-ylamido. In certain embodiments, in Formula (IV), (IVa), or (IVb), R^{7d} is hydrogen; and R^{7c} is methyl, hydroxycarbonylmethyl, ethoxycarbonylmethyl, ethyl, 2-hydroxyethyl, 2-methoxyethyl, 2-acetoxyethyl, 2-aminoethyl, 2-acetamidoethyl, 2-(2-aminoacetamido)ethyl, 2-(2-aminopropanamido)ethyl, 2-(2-amino-3-hydroxypropanamido)ethyl, 2-(2-amino-3-methylbutanamido)ethyl, 2-(2-amino-4-(methylthio)-butanamido)ethyl, 2-(2-amino-4-methylpentanamido)ethyl, 2-(pyrrolidin-2-ylamido)ethyl, isopropyl, tert-butyl, cyclopropyl, cyclobutyl, or tetrahydropyran-4-yl. In certain embodiments, in Formula (IV), (IVa), or (IVb), R^{7d} is hydrogen; and R^{7c} is methyl, hydroxycarbonylmethyl, ethoxycarbonylmethyl, ethyl, 2-hydroxyethyl, 2-methoxyethyl, 2-acetoxyethyl, 2-aminoethyl, 2-acetamidoethyl, 2-(2-aminoacetamido)ethyl, (R)-2-(2-aminopropanamido)ethyl, (S)-2-(2-amino-3-hydroxypropanamido)ethyl, (S)-2-(2-amino-3-methylbutanamido)ethyl, (S)-2-(2-amino-4-(methylthio)butanamido)ethyl, (S)-2-(2-amino-4-methylpentanamido)ethyl, (S)-2-(pyrrolidin-2-ylamido)ethyl, isopropyl, tert-butyl, cyclopropyl, cyclobutyl, or tetrahydropyran-4-yl.

In yet another embodiment, provided herein is a compound of Formula (V):

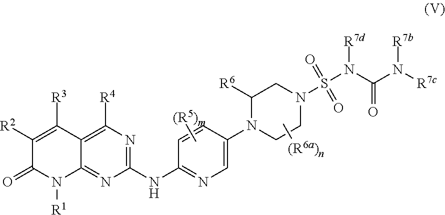

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R¹, R², R³, R⁴, R⁵, R⁶, R^{6a}, R^{7b}, R^{7c}, R^{7d}, m, and n are each as defined herein.

In yet another embodiment, provided herein is a compound of Formula (Va):

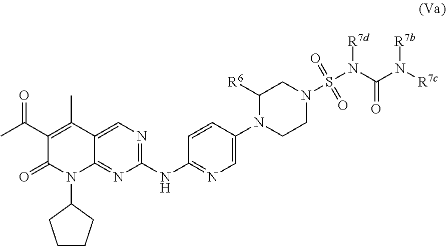

or an enantiomer, a mixture of enantiomers, a diastereomer, a mixture of two or more or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶, R^{7b}, R^{7c}, and R^{7d} are each as defined herein.

In yet another embodiment, provided herein is a compound of Formula (Vb):

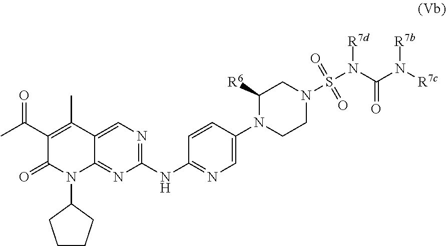

or an enantiomer, a mixture of enantiomers, a diastereomer, a mixture of two or more or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶, R^{7b}, R^{7c}, and R^{7d} are each as defined herein.

In certain embodiments, in Formula (V), (Va), or (Vb), R⁶ is hydrogen or C₁₋₆ alkyl, optionally substituted with one or more substituents Q. In certain embodiments, in Formula (V), (Va), or (Vb), R⁶ is hydrogen or methyl. In certain embodiments, in Formula (V), (Va), or (Vb), R⁶ is hydrogen. In certain embodiments, in Formula (V), (Va), or (Vb), R⁶ is methyl.

In certain embodiments, in Formula (V), (Va), or (Vb), R^{7b} and R^{7d} are each hydrogen; and R^{7c} is C₁₋₆ alkyl, optionally substituted with one or more substituents Q. In certain embodiments, in Formula (V), (Va), or (Vb), R^{7b} and R^{7d} are each hydrogen; and R^{7c} is isopropyl.

In yet another embodiment, provided herein is a compound of Formula (VI):

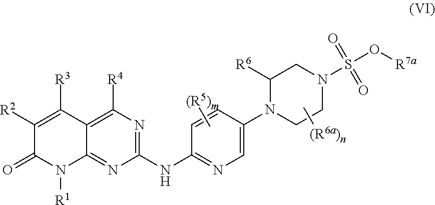

or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R¹, R², R³, R⁴, R⁵, R⁶, R^{6a}, R^{7a}, m, and n are each as defined herein.

In yet another embodiment, provided herein is a compound of Formula (VIa):

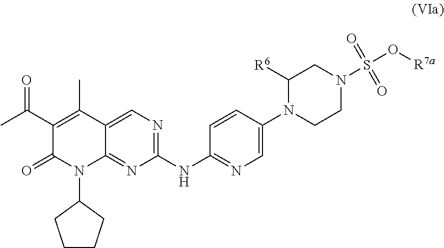

or an enantiomer, a mixture of enantiomers, a diastereomer, a mixture of two or more or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶ and R^{7a} are each as defined herein.

In still another embodiment, provided herein is a compound of Formula (VIb):

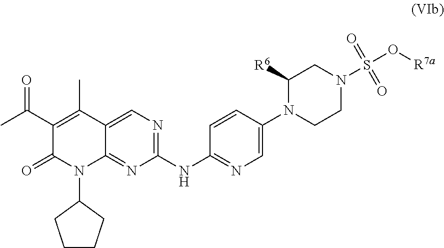

or an enantiomer, a mixture of enantiomers, a diastereomer, a mixture of two or more or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein R⁶ and R^{7a} are each as defined herein.

In certain embodiments, in Formula (VI), (VIa), or (VIb), R⁶ is hydrogen or C₁₋₆ alkyl, optionally substituted with one or more substituents Q. In certain embodiments, in Formula (VI), (VIa), or (VIb), R⁶ is hydrogen or methyl. In certain embodiments, in Formula (V), (Va), or (Vb), R⁶ is hydrogen. In certain embodiments, in Formula (VI), (VIa), or (VIb), R⁶ is methyl.

In certain embodiments, in Formula (VI), (VIa), or (VIb), R^{7a} is C₁₋₆ alkyl, C₃₋₁₀ cycloalkyl, or C₆₋₁₄ aryl, each optionally substituted with one or more substituents Q. In certain embodiments, in Formula (VI), (VIa), or (VIb), R^{7a} is C₁₋₆ alkyl, optionally substituted with one, two, or three substituents Q. In certain embodiments, in Formula (VI), (VIa), or (VIb), R^{7a} is methyl, optionally substituted with one or more substituents Q. In certain embodiments, in Formula (VI), (VIa), or (VIb), R^{7a} is methyl, optionally substituted with —OC(O)R^{1a}, wherein R^{1a} is as defined herein. In certain embodiments, in Formula (VI), (VIa), or (VIb), R^{7a} is C₃₋₁₀ cycloalkyl, optionally substituted with one, two, or three substituents Q. In certain embodiments, in Formula (VI), (VIa), or (VIb), R^{7a} is monocyclic C₃₋₁₀ cycloalkyl, optionally substituted with one, two, or three substituents Q. In certain embodiments, in Formula (VI), (VIa), or (VIb), R^{7a} is C₆₋₁₄ aryl, optionally substituted with one, two, or three substituents Q. In certain embodiments, in Formula (VI), (VIa), or (VIb), R^{7a} is methyl, ethyl, propyl, cyclopentyl, or phenyl, each optionally substituted with one, two, or three substituents Q. In certain embodiments, in Formula (VI), (Via), or (VIb), R^{7a} is pivaloyloxymethyl, benzoyloxymethyl, isopropyl, cyclopentyl, or 4-methylphenyl.

The groups, R¹, R², R³, R⁴, R⁵, R⁶, R⁷, R^{6a}, R^{7a}, R^{7b}, R^{7c}, R^{7d}, R^{7e}, m, and n, in formulae described herein, including Formulae (I) to (VI), (Ia) to (VIa), and (Ib) to (VIb), are further defined in the embodiments described herein. All combinations of the embodiments provided herein for such groups are within the scope of this disclosure.

In certain embodiments, R¹ is hydrogen. In certain embodiments, R¹ is isopropyl. In certain embodiments, R¹ is C₁₋₆ heteroalkyl, optionally substituted with one or more substituents Q. In certain embodiments, R¹ is C₂₋₆ alkenyl, optionally substituted with one or more substituents Q. In certain embodiments, R¹ is C₂₋₆ alkynyl, optionally substituted with one or more substituents Q. In certain embodiments, R¹ is C₃₋₁₀ cycloalkyl, optionally substituted with one or more substituents Q. In certain embodiments, R¹ is monocyclic C₃₋₁₀ cycloalkyl, optionally substituted with one or more substituents Q. In certain embodiments, R¹ is cyclopropyl, cyclobutyl, cyclopentyl, or cyclohexyl, each optionally substituted with one or more substituents Q. In certain embodiments, R¹ is cyclopropyl, cyclobutyl, cyclopentyl, or cyclohexyl. In certain embodiments, R¹ is bicyclic C₄₋₁₀ cycloalkyl, optionally substituted with one or more substituents Q. In certain embodiments, R¹ is bridged, fused, or spiro C₄₋₁₀ cycloalkyl, each optionally substituted with one or more substituents Q. In certain embodiments, R¹ is C₆₋₁₄ aryl, optionally substituted with one or more substituents Q. In certain embodiments, R¹ is C₇₋₁₅ aralkyl, optionally substituted with one or more substituents Q. In certain embodiments, R¹ is heteroaryl, optionally substituted with one or more substituents Q. In certain embodiments, R¹ is heterocyclyl, optionally substituted with one or more substituents Q. In certain embodiments, R¹ is —C(O)R^{1a}, wherein R^{1a} is as defined herein. In certain embodiments, R¹ is —C(O)OR^{1a}, wherein R^{1a} is as defined herein. In certain embodiments, R¹ is —C(O)NR^{1b}R^{1c}, wherein R^{1b} and R^{1c} are each as defined herein. In certain embodiments, R¹ is —C(NR^{1a})NR^{1b}R^{1c}, wherein R^{1a}, R^{1b}, and R^{1c} are each as defined herein.

In certain embodiments, R¹ is C₁₋₆ alkyl or C₃₋₁₀ cycloalkyl, each optionally substituted with one or more substituents Q. In certain embodiments, R¹ is C₁₋₆ alkyl or monocyclic C₃₋₁₀ cycloalkyl, each optionally substituted with one or more substituents Q. In certain embodiments, R¹ is isopropyl, cyclopentyl, or cyclohexyl. In certain embodiments, R¹ is cyclopentyl.

In certain embodiments, R² is hydrogen. In certain embodiments, R² is deuterium. In certain embodiments, R² is cyano. In certain embodiments, R² is halo. In certain embodiments, R² is fluoro, chloro, bromo, or iodo. In certain embodiments, R² is nitro. In certain embodiments, R² is C₁₋₆ alkyl, optionally substituted with one or more substituents Q. In certain embodiments, R² is methyl, ethyl, isopropyl, hydroxymethyl, or ethoxymethyl. In certain embodiments, R² is C₁₋₆ heteroalkyl, optionally substituted with one or more substituents Q. In certain embodiments, R² is C₂₋₆ alkenyl, optionally substituted with one or more substituents Q. In certain embodiments, R² is C₂₋₆ alkynyl, optionally substituted with one or more substituents Q. In certain embodiments, R² is C₃₋₁₀ cycloalkyl, optionally substituted with one or more substituents Q. In certain embodiments, R² is C₆₋₁₄ aryl, optionally substituted with one or more substituents Q. In certain embodiments, R² is C₇₋₁₅ aralkyl, optionally substituted with one or more substituents Q. In certain embodiments, R² is benzyl, optionally substituted with one or more substituents Q. In certain embodiments, R² is heteroaryl, optionally substituted with one or more substituents Q. In certain embodiments, R² is heterocyclyl, optionally substituted with one or more substituents Q.

In certain embodiments, R² is —C(O)R^{1a}, wherein R^{1a} is as defined herein. In certain embodiments, R² is —C(O)—C₁₋₆ alkyl, optionally substituted with one or more substituents Q. In certain embodiments, R² is acetyl. In certain embodiments, R² is —C(O)OR^{1a}, wherein R^{1a} is as defined herein. In certain embodiments, R² is —COOH or —C(O)O—C₁₋₆ alkyl, optionally substituted with one or more substituents Q. In certain embodiments, R² is —COOH or —COOEt. In certain embodiments, R² is —C(O)NR^{1b}R^{1c}, wherein R^{1b} and R^{1c} are each as defined herein. In certain embodiments, R² is —C(O)SR^{1a}, wherein R^{1a} is as defined herein. In certain embodiments, R² is —C(NR^{1a})NR^{1b}R^{1c}, wherein R^{1a}, R^{1b}, and R^{1c} are each as defined herein. In certain embodiments, R² is —C(S)R^{1a}, wherein R^{1a} is as defined herein. In certain embodiments, R² is —C(S)OR^{1a}, wherein R^{1a} is as defined herein. In certain embodiments, R² is —C(S)NR^{1b}R^{1c}, wherein R^{1b} and R^{1c} are each as defined herein. In certain embodiments, R² is —OR^{1a}, wherein R^{1a} is as defined herein. In certain embodiments, R² is C₁₋₆ alkoxy, optionally substituted with one or more substituents Q. In certain embodiments, R² is 2-ethoxyethoxy. In certain embodiments, R² is —OC(O)R^{1a}, wherein R^{1a} is as defined herein. In certain embodiments, R² is —OC(O)OR^{1a}, wherein R^{1a} is as defined herein. In certain embodiments, R² is —OC(O)NR^{1b}R^{1c}, wherein R^{1b} and R^{1c} are each as defined herein. In certain embodiments, R² is —OC(S)R^{1a}, wherein R^{1a} is as defined herein. In certain embodiments, R² is —OC(NR^{1a})NR^{1b}R^{1c}, wherein R^{1a}, R^{1b}, and R^{1c} are each as defined herein. In certain embodiments, R² is —OC(S)R^{1a}, wherein R^{1a} is as defined herein. In certain embodiments, R² is —OC(S)OR^{1a}, wherein R^{1a} is as defined herein. In certain embodiments, R² is —OC(S)NR^{1b}R^{1c}, wherein R^{1b} and R^{1c} are each as defined herein. In certain embodiments, R² is —OS(O)R^{1a}, wherein R^{1a} is as defined herein. In certain embodiments, R² is —OS(O)₂R^{1a}, wherein R^{1a} is as defined herein. In certain embodiments, R² is —OS(O)NR^{1b}R^{1c}, wherein R^{1b} and R^{1c} are each as defined herein. In certain embodiments, R² is —OS(O)₂NR^{1b}R^{1c}, wherein R^{1b} and R^{1c} are each as defined herein. In certain embodiments, R² is —NR^{1b}R^{1c}, wherein R^{1b} and R^{1c} are each as defined herein. In certain embodiments, R² is —NH₂. In certain embodiments, R² is —NR^{1a}C(O)Rid, wherein R^{1a} and R^{1d} are each as defined herein. In certain embodiments, R² is —NR^{1a}C(O)OR^{1d}, wherein R^{1a} and R^{1d} are each as defined herein. In certain embodiments, R² is —NR^{1a}C(O)NR^{1b}R^{1c}, wherein R^{1a}, R^{1b}, and R^{1c} are each as defined herein. In certain embodiments, R² is —NR^{1a}C(O)SR^{1d}, wherein R^{1a} and R^{1d} are each as defined herein. In certain embodiments, R² is —NR^{1a}C(NR^{1d})NR^{1b}R^{1c}, wherein R^{1a}, R^{1b}, R^{1c}, and R^{1d} are each as defined herein. In certain embodiments, R² is —NR^{1a}C(S)R^{1d}, wherein R^{1a} and R^{1d} are each as defined herein. In certain embodiments, R² is —NR^{1a}C(S)OR^{1d}, wherein R^{1a} and R^{1d} are each as defined herein. In certain embodiments, R² is —NR^{1a}C(S)NR^{1b}R^{1c}, wherein R^{1a}, R^{1b}, and R^{1c} are each as defined herein. In certain embodiments, R² is —NR^{1a}S(O)R^{1d}, wherein R^{1a} and R^{1d} are each as defined herein. In certain embodiments, R² is —NR^{1a}S(O)₂R^{1d}, wherein R^{1a} and R^{1d} are each as defined herein. In certain embodiments, R² is —NR^{1a}S(O)NR^{1b}R^{1c}, wherein R^{1a}, R^{1b}, and R^{1c} are each as defined herein. In certain embodiments, R² is —NR^{1a}S(O)₂NR^{1b}R^{1c}, wherein R^{1a}, R^{1b}, and R^{1c} are each as defined herein. In certain embodiments, R² is —SR^{1a}, wherein R^{1a} is as defined herein. In certain embodiments, R² is —S(O)R^{1a}, wherein R^{1a} is as defined herein. In certain embodiments, R² is —S(O)₂R^{1a}, wherein R^{1a} is as defined herein. In certain embodiments, R² is —S(O)NR^{1b}R^{1c}, wherein R^{1b} and R^{1c} are each as defined herein. In certain embodiments, R² is —S(O)₂NR^{1b}R^{1c}, wherein R^{1b} and R^{1c} are each as defined herein.

In certain embodiments, R² is (i) halo; (ii) C₁₋₆ alkyl or C₇₋₁₅ aralkyl, each optionally substituted with one or more substituents Q; or (iii) —C(O)R^{1a}, —C(O)OR^{1a}, —OR^{1a}, or —NR^{1b}R^{1c}, wherein each R^{1d}, R^{1b}, and R^{1c} is as defined herein. In certain embodiments, R² is fluoro, chloro, bromo, iodo, methyl, ethyl, isopropyl, hydroxymethyl, ethoxymethyl, benzyl, acetyl, hydroxy-carbonyl, ethoxycarbonyl, 2-ethoxyethoxy, or amino. In certain embodiments, R² is acetyl.

In certain embodiments, R³ is hydrogen. In certain embodiments, R³ is deuterium. In certain embodiments, R³ is cyano. In certain embodiments, R³ is halo. In certain embodiments, R³ is fluoro or chloro. In certain embodiments, R³ is nitro. In certain embodiments, R³ is C₁₋₆ alkyl, optionally substituted with one or more substituents Q. In certain embodiments, R³ is methyl. In certain embodiments, R³ is C₁₋₆ heteroalkyl, optionally substituted with one or more substituents Q. In certain embodiments, R³ is trifluoromethyl. In certain embodiments, R³ is C₂₋₆ alkenyl, optionally substituted with one or more substituents Q. In certain embodiments, R³ is C₂₋₆ alkynyl, optionally substituted with one or more substituents Q. In certain embodiments, R³ is C₃₋₁₀ cycloalkyl, optionally substituted with one or more substituents Q. In certain embodiments, R³ is C₆₋₁₄ aryl, optionally substituted with one or more substituents Q. In certain embodiments, R³ is C₇₋₁₅ aralkyl, optionally substituted with one or more substituents Q. In certain embodiments, R³ is heteroaryl, optionally substituted with one or more substituents Q. In certain embodiments, R³ is heterocyclyl, optionally substituted with one or more substituents Q. In certain embodiments, R³ is hydrogen or methyl.

In certain embodiments, R³ is —C(O)R^{1a}, wherein R^{1a} is as defined herein. In certain embodiments, R³ is —C(O)OR^{1a}, wherein R^{1a} is as defined herein. In certain embodiments, R³ is —C(O)NR^{1b}R^{1c}, wherein R^{1b} and R^{1c} are each as defined herein. In certain embodiments, R³ is —C(O)SR^{ia}, wherein R^{1a} is as defined herein. In certain embodiments, R³ is —C(NR^{1a})NR^{1b}R^{1c}, wherein R^{1a}, R^{1b}, and R^{1c} are each as defined herein. In certain embodiments, R³ is —C(S)R^{1a}, wherein R^{1a} is as defined herein. In certain embodiments, R³ is —C(S)OR^{1a}, wherein R^{1a} is as defined herein. In certain embodiments, R³ is —C(S)NR^{1b}R^{1c}, wherein R^{1b} and R^{1c} are each as defined herein. In certain embodiments, R³ is —OR^{1a}, wherein R^{1a} is as defined herein. In certain embodiments, R³ is —OC(O)R^{1a}, wherein R^{1a} is as defined herein. In certain embodiments, R³ is —OC(O)OR^{1a}, wherein R^{1a} is as defined herein. In certain embodiments, R³ is —OC(O)NR^{1b}R^{1c}, wherein R^{1b} and R^{1c} are each as defined herein. In certain embodiments, R³ is —OC(S)R^{1a}, wherein R^{1a} is as defined herein. In certain embodiments, R³ is —OC(NR^{1a})NR^{1b}R^{1c}, wherein R^{1a}, R^{1b}, and R^{1c} are each as defined herein. In certain embodiments, R³ is —OC(S)R^{1a}, wherein R^{1a} is as defined herein. In certain embodiments, R³ is —OC(S)OR^{1a}, wherein R^{1a} is as defined herein. In certain embodiments, R³ is —OC(S)NR^{1b}R^{1c}, wherein R^{1b} and R^{1c} are each as defined herein. In certain embodiments, R³ is —OS(O)R^{1a}, wherein R^{1a} is as defined herein. In certain embodiments, R³ is —OS(O)₂R^{1a}, wherein R^{1a} is as defined herein. In certain embodiments, R³ is —OS(O)NR^{1b}R^{1c}, wherein R^{1b} and R^{1c} are each as defined herein. In certain embodiments, R³ is —OS(O)₂NR^{1b}R^{1c}, wherein R^{1b} and R^{1c} are each as defined herein. In certain embodiments, R³ is —NR^{1b}R^{1c}, wherein R^{1b} and R^{1c} are each as defined herein. In certain embodiments, R³ is —NR^{1a}C(O)R^{1d}, wherein R^{1a} and R^{1d} are each as defined herein. In certain embodiments, R³ is —NR^{1a}C(O)OR^{1d}, wherein R^{1a} and R^{1d} are each as defined herein. In certain embodiments, R³ is —NR^{1a}C(O)NR^{1b}R^{1c}, wherein R^{1a}, R^{1b}, and R^{1c} are each as defined herein. In certain embodiments, R³ is —NR^{1a}C(O)SR^{1d}, wherein R^{1a} and R^{1d} are each as defined herein. In certain embodiments, R³ is —NR^{1a}C(NR^{1d})NR^{1b}R^{1c}, wherein R^{1a}, R^{1b}, R^{1c}, and R^{1d} are each as defined herein. In certain embodiments, R³ is —NR^{1a}C(S)R^{1d}, wherein R^{1a} and R^{1d} are each as defined herein. In certain embodiments, R³ is —NR^{1a}C(S)OR^{1d}, wherein R^{1a} and R^{1d} are each as defined herein. In certain embodiments, R³ is —NR^{1a}C(S)NR^{1b}R^{1c}, wherein R^{1a}, R^{1b}, and R^{1c} are each as defined herein. In certain embodiments, R³ is —NR^{1a}S(O)R^{1d}, wherein R^{1a} and R^{1d} are each as defined herein. In certain embodiments, R³ is —NR^{1a}S(O)₂R^{1d}, wherein R^{1a} and R^{1d} are each as defined herein. In certain embodiments, R³ is —NR^{1a}S(O)NR^{1b}R^{1c}, wherein R^{1a}, R^{1b}, and R^{1c} are each as defined herein. In certain embodiments, R³ is —NR^{1a}S(O)₂NR^{1b}R^{1c}, wherein R^{1a}, R^{1b}, and R^{1c} are each as defined herein. In certain embodiments, R³ is —SR^{1a}, wherein R^{1a} is as defined herein. In certain embodiments, R³ is —S(O)R^{1a}, wherein R^{1a} is as defined herein. In certain embodiments, R³ is —S(O)₂R^{1a}, wherein R^{1a} is as defined herein. In certain embodiments, R³ is —S(O)NR^{1b}R^{1c}, wherein R^{1b} and R^{1c} are each as defined herein. In certain embodiments, R³ is —S(O)₂NR^{1b}R^{1c}, wherein R^{1b} and R^{1c} are each as defined herein.

In certain embodiments, R⁴ is hydrogen. In certain embodiments, R⁴ is deuterium. In certain embodiments, R⁴ is cyano. In certain embodiments, R⁴ is halo. In certain embodiments, R⁴ is fluoro. In certain embodiments, R⁴ is nitro. In certain embodiments, R⁴ is C₁₋₆ alkyl, optionally substituted with one or more substituents Q. In certain embodiments, R⁴ is C₁₋₆ heteroalkyl, optionally substituted with one or more substituents Q. In certain embodiments, R⁴ is C₂₋₆ alkenyl, optionally substituted with one or more substituents Q. In certain embodiments, R⁴ is C₂₋₆ alkynyl, optionally substituted with one or more substituents Q. In certain embodiments, R⁴ is C₃₋₁₀ cycloalkyl, optionally substituted with one or more substituents Q. In certain embodiments, R⁴ is C₆₋₁₄ aryl, optionally substituted with one or more substituents Q. In certain embodiments, R⁴ is C₇₋₁₅ aralkyl, optionally substituted with one or more substituents Q. In certain embodiments, R⁴ is heteroaryl, optionally substituted with one or more substituents Q. In certain embodiments, R⁴ is heterocyclyl, optionally substituted with one or more substituents Q.

In certain embodiments, R⁴ is —C(O)R^{1a}, wherein R^{1a} is as defined herein. In certain embodiments, R⁴ is —C(O)OR^{1a}, wherein R^{1a} is as defined herein. In certain embodiments, R⁴ is —C(O)NR^{1b}R^{1c}, wherein R^{1b} and R^{1c} are each as defined herein. In certain embodiments, R⁴ is —C(O)SR^{1a}, wherein R^{1a} is as defined herein. In certain embodiments, R⁴ is —C(NR^{1a})NR^{1b}R^{1c}, wherein R^{1a}, R^{1b}, and R^{1c} are each as defined herein. In certain embodiments, R⁴ is —C(S)R^{1a}, wherein R^{1a} is as defined herein. In certain embodiments, R⁴ is —C(S)OR^{1a}, wherein R^{1a} is as defined herein. In certain embodiments, R⁴ is —C(S)NR^{1b}R^{1c}, wherein R^{1b} and R^{1c} are each as defined herein. In certain embodiments, R⁴ is —OR^{1a}, wherein R^{1a} is as defined herein. In certain embodiments, R⁴ is —OC(O)R^{1a}, wherein R^{1a} is as defined herein. In certain embodiments, R⁴ is —OC(O)OR^{1a}, wherein R^{1a} is as defined herein. In certain embodiments, R⁴ is —OC(O)NR^{1b}R^{1c}, wherein R^{1b} and R^{1c} are each as defined herein. In certain embodiments, R⁴ is —OC(S)R^{1a}, wherein R^{1a} is as defined herein. In certain embodiments, R⁴ is —OC(NR^{1a})NR^{1b}R^{1c}, wherein R^{1a}, R^{1b}, and R^{1c} are each as defined herein. In certain embodiments, R⁴ is —OC(S)R^{1a}, wherein R^{1a} is as defined herein. In certain embodiments, R⁴ is —OC(S)OR^{1a}, wherein R^{1a} is as defined herein. In certain embodiments, R⁴ is —OC(S)NR^{1b}R^{1c}, wherein R^{1b} and R^{1c} are each as defined herein. In certain embodiments, R⁴ is —OS(O)R^{1a}, wherein R^{1a} is as defined herein. In certain embodiments, R⁴ is —OS(O)₂R^{1a}, wherein R^{1a} is as defined herein. In certain embodiments, R⁴ is —OS(O)NR^{1b}R^{1c}, wherein R^{1b} and R^{1c} are each as defined herein. In certain embodiments, R⁴ is —OS(O)₂NR^{1b}R^{1c}, wherein R^{1b} and R^{1c} are each as defined herein. In certain embodiments, R⁴ is —NR^{1b}R^{1c}, wherein R^{1b} and R^{1c} are each as defined herein. In certain embodiments, R⁴ is —NR^{1a}C(O)R^{1d}, wherein R^{1a} and R^{1d} are each as defined herein. In certain embodiments, R⁴ is —NR^{1a}C(O)OR^{1d}, wherein R^{1a} and R^{1d} are each as defined herein. In certain embodiments, R⁴ is —NR^{1a}C(O)NR^{1b}R^{1c}, wherein R^{1a}, R^{1b}, and R^{1c} are each as defined herein. In certain embodiments, R⁴ is —NR^{1a}C(O)SR^{1d}, wherein R^{1a} and R^{1d} are each as defined herein. In certain embodiments, R⁴ is —NR^{1a}C(NR^{1d})NR^{1b}R^{1c}, wherein R^{1a}, R^{1b}, R^{1c}, and R^{1d} are each as defined herein. In certain embodiments, R⁴ is —NR^{1a}C(S)R^{1d}, wherein R^{1a} and R^{1d} are each as defined herein. In certain embodiments, R⁴ is —NR^{1a}C(S)OR^{1d}, wherein R^{1a} and R^{1d} are each as defined herein. In certain embodiments, R⁴ is —NR^{1a}C(S)NR^{1b}R^{1c}, wherein R^{1a}, R^{1b}, and R^{1c} are each as defined herein. In certain embodiments, R⁴ is —NR^{1a}S(O)R^{1d}, wherein R^{1a} and R^{1d} are each as defined herein. In certain embodiments, R⁴ is —NR^{1a}S(O)₂R^{1d}, wherein R^{1a} and R^{1d} are each as defined herein. In certain embodiments, R⁴ is —NR^{1a}S(O)NR^{1b}R^{1c}, wherein R^{1d}, R^{1b}, and R^{1c} are each as defined herein. In certain embodiments, R⁴ is —NR^{1a}S(O)₂NR^{1b}R^{1c}, wherein R^{1a}, R^{1b}, and R^{1c} are each as defined herein. In certain embodiments, R⁴ is —SR^{1a}, wherein R^{1a} is as defined herein. In certain embodiments, R⁴ is —S(O)R^{1a}, wherein R^{1a} is as defined herein. In certain embodiments, R⁴ is —S(O)₂R^{1a}, wherein R^{1a} is as defined herein. In certain embodiments, R⁴ is —S(O)NR^{1b}R^{1c}, wherein R^{1b} and R^{1c} are each as defined herein. In certain embodiments, R⁴ is —S(O)₂NR^{1b}R^{1c}, wherein R^{1b} and R^{1c} are each as defined herein.

In certain embodiments, R⁵ is deuterium. In certain embodiments, R⁵ is cyano. In certain embodiments, R⁵ is halo. In certain embodiments, R⁵ is fluoro or chloro. In certain embodiments, R⁵ is nitro. In certain embodiments, R⁵ is C₁₋₆ alkyl, optionally substituted with one or more substituents Q. In certain embodiments, R⁵ is methyl or ethyl. In certain embodiments, R⁵ is C₁₋₆ heteroalkyl, optionally substituted with one or more substituents Q. In certain embodiments, R⁵ is C₂₋₆ alkenyl, optionally substituted with one or more substituents Q. In certain embodiments, R⁵ is C₂₋₆ alkynyl, optionally substituted with one or more substituents Q. In certain embodiments, R⁵ is C₃₋₁₀ cycloalkyl, optionally substituted with one or more substituents Q. In certain embodiments, R⁵ is C₆₋₁₄ aryl, optionally substituted with one or more substituents Q. In certain embodiments, R⁵ is C₇₋₁₅ aralkyl, optionally substituted with one or more substituents Q. In certain embodiments, R⁵ is heteroaryl, optionally substituted with one or more substituents Q. In certain embodiments, R⁵ is heterocyclyl, optionally substituted with one or more substituents Q. In certain embodiments, R⁵ is halo or C₁₋₆ alkyl, optionally substituted with one, two, or three substituents Q. In certain embodiments, R⁵ is fluoro, chloro, methyl, or ethyl.

In certain embodiments, R⁵ is —C(O)R^{1a}, wherein R^{1a} is as defined herein. In certain embodiments, R⁵ is —C(O)OR^{1a}, wherein R^{1a} is as defined herein. In certain embodiments, R⁵ is —C(O)NR^{1b}R^{1c}, wherein R^{1b} and R^{1c} are each as defined herein. In certain embodiments, R⁵ is —C(O)SR^{1a}, wherein R^{1a} is as defined herein. In certain embodiments, R⁵ is —C(NR^{1a})NR^{1b}R^{1c}, wherein R^{1a}, R^{1b}, and R^{1c} are each as defined herein. In certain embodiments, R⁵ is —C(S)R^{1a}, wherein R^{1a} is as defined herein. In certain embodiments, R⁵ is —C(S)OR^{1a}, wherein R^{1a} is as defined herein. In certain embodiments, R⁵ is —C(S)NR^{1b}R^{1c}, wherein R^{1b} and R^{1c} are each as defined herein. In certain embodiments, R⁵ is —OR^{1a}, wherein R^{1a} is as defined herein. In certain embodiments, R⁵ is —OC(O)R^{1a}, wherein R^{1a} is as defined herein. In certain embodiments, R⁵ is —OC(O)OR^{1a}, wherein R^{1a} is as defined herein. In certain embodiments, R⁵ is —OC(O)NR^{1b}R^{1c}, wherein R^{1b} and R^{1c} are each as defined herein. In certain embodiments, R⁵ is —OC(S)R^{1a}, wherein R^{1a} is as defined herein. In certain embodiments, R⁵ is —OC(NR^{1a})NR^{1b}R^{1c}, wherein R^{1a}, R^{1b}, and R^{1c} are each as defined herein. In certain embodiments, R⁵ is —OC(S)R^{1a}, wherein R^{1a} is as defined herein. In certain embodiments, R⁵ is —OC(S)OR^{1a}, wherein R^{1a} is as defined herein. In certain embodiments, R⁵ is —OC(S)NR^{1b}R^{1c}, wherein R^{1b} and R^{1c} are each as defined herein. In certain embodiments, R⁵ is —OS(O)R^{1a}, wherein R^{1a} is as defined herein. In certain embodiments, R⁵ is —OS(O)₂R^{1a}, wherein R^{1a} is as defined herein. In certain embodiments, R⁵ is —OS(O)NR^{1b}R^{1c}, wherein R^{1b} and R^{1c} are each as defined herein. In certain embodiments, R⁵ is —OS(O)₂NR^{1b}R^{1c}, wherein R^{1b} and R^{1c} are each as defined herein. In certain embodiments, R⁵ is —NR^{1b}R^{1c}, wherein R^{1b} and R^{1c} are each as defined herein. In certain embodiments, R⁵ is —NR^{1a}C(O)R^{1d}, wherein R^{1a} and R^{1d} are each as defined herein. In certain embodiments, R⁵ is —NR^{1a}C(O)OR^{1d}, wherein R^{1a} and R^{1d} are each as defined herein. In certain embodiments, R⁵ is —NR^{1a}C(O)NR^{1b}R^{1c}, wherein R^{1a}, R^{1b}, and R^{1c} are each as defined herein. In certain embodiments, R⁵ is —NR^{1a}C(O)SR^{1d}, wherein R^{1a} and R^{1d} are each as defined herein. In certain embodiments, R⁵ is —NR^{1a}C(NR^{1d})NR^{1b}R^{1c}, wherein R^{1a}, R^{1b}, R^{1c}, and R^{1d} are each as defined herein. In certain embodiments, R⁵ is —NR^{1a}C(S)R^{1d}, wherein R^{1a} and R^{1d} are each as defined herein. In certain embodiments, R⁵ is —NR^{1a}C(S)OR^{1d}, wherein R^{1a} and R^{1d} are each as defined herein. In certain embodiments, R⁵ is —NR^{1a}C(S)NR^{1b}R^{1c}, wherein R^{1a}, R^{1b}, and R^{1c} are each as defined herein. In certain embodiments, R⁵ is —NR^{1a}S(O)R^{1d}, wherein R^{1a} and R^{1d} are each as defined herein. In certain embodiments, R⁵ is —NR^{1a}S(O)₂R^{1d}, wherein R^{1a} and R^{1d} are each as defined herein. In certain embodiments, R⁵ is —NR^{1a}S(O)NR^{1b}R^{1c}, wherein R^{1a}, R^{1b}, and R^{1c} are each as defined herein. In certain embodiments, R⁵ is —NR^{1a}S(O)₂NR^{1b}R^{1c}, wherein R^{1a}, R^{1b}, and R^{1c} are each as defined herein. In certain embodiments, R⁵ is —SR^{1a}, wherein R^{1a} is as defined herein. In certain embodiments, R⁵ is —S(O)R^{1a}, wherein R^{1a} is as defined herein. In certain embodiments, R⁵ is —S(O)₂R^{1a}, wherein R^{1a} is as defined herein. In certain embodiments, R⁵ is —S(O)NR^{1b}R^{1c}, wherein R^{1b} and R^{1c} are each as defined herein. In certain embodiments, R⁵ is —S(O)₂NR^{1b}R^{1c}, wherein R^{1b} and R^{1c} are each as defined herein.

In certain embodiments, R⁶ is hydrogen. In certain embodiments, R⁶ is deuterium. In certain embodiments, R⁶ is cyano. In certain embodiments, R⁶ is halo. In certain embodiments, R⁶ is fluoro. In certain embodiments, R⁶ is nitro. In certain embodiments, R⁶ is C₁₋₆ alkyl, optionally substituted with one or more substituents Q. In certain embodiments, R⁶ is methyl. In certain embodiments, R⁶ is C₁₋₆ heteroalkyl, optionally substituted with one or more substituents Q. In certain embodiments, R⁶ is trifluoromethyl. In certain embodiments, R⁶ is C₂₋₆ alkenyl, optionally substituted with one or more substituents Q. In certain embodiments, R⁶ is C₂₋₆ alkynyl, optionally substituted with one or more substituents Q. In certain embodiments, R⁶ is C₃₋₁₀ cycloalkyl, optionally substituted with one or more substituents Q. In certain embodiments, R⁶ is C₆₋₁₄ aryl, optionally substituted with one or more substituents Q. In certain embodiments, R⁶ is C₇₋₁₅ aralkyl, optionally substituted with one or more substituents Q. In certain embodiments, R⁶ is heteroaryl, optionally substituted with one or more substituents Q. In certain embodiments, R⁶ is heterocyclyl, optionally substituted with one or more substituents Q. In certain embodiments, R⁶ is hydrogen or methyl.

In certain embodiments, R⁶ is —C(O)R^{1a}, wherein R^{1a} is as defined herein. In certain embodiments, R⁶ is —C(O)OR^{1a}, wherein R^{1a} is as defined herein. In certain embodiments, R⁶ is —C(O)NR^{1b}R^{1c}, wherein R^{1b} and R^{1c} are each as defined herein. In certain embodiments, R⁶ is —C(O)SR^{1a}, wherein R^{1a} is as defined herein. In certain embodiments, R⁶ is —C(NR^{1a})NR^{1b}R^{1c}, wherein R^{1a}, R^{1b}, and R^{1c} are each as defined herein. In certain embodiments, R⁶ is —C(S)R^{1a}, wherein R^{1a} is as defined herein. In certain embodiments, R⁶ is —C(S)OR^{1a}, wherein R^{1a} is as defined herein. In certain embodiments, R⁶ is —C(S)NR^{1b}R^{1c}, wherein R^{1b} and R^{1c} are each as defined herein. In certain embodiments, R⁶ is —OR^{1a}, wherein R^{1a} is as defined herein. In certain embodiments, R⁶ is —OC(O)R^{1a}, wherein R^{1a} is as defined herein. In certain embodiments, R⁶ is —OC(O)OR^{1a}, wherein R^{1a} is as defined herein. In certain embodiments, R⁶ is —OC(O)NR^{1b}R^{1c}, wherein R^{1b} and R^{1c} are each as defined herein. In certain embodiments, R⁶ is —OC(S)R^{1a}, wherein R^{1a} is as defined herein. In certain embodiments, R⁶ is —OC(NR^{1a})NR^{1b}R^{1c}, wherein R^{1a}, R^{1b}, and R^{1c} are each as defined herein. In certain embodiments, R⁶ is —OC(S)R^{1a}, wherein R^{1a} is as defined herein. In certain embodiments, R⁶ is —OC(S)OR^{1a}, wherein R^{1a} is as defined herein. In certain embodiments, R⁶ is —OC(S)NR^{1b}R^{1c}, wherein R^{1b} and R^{1c} are each as defined herein. In certain embodiments, R⁶ is —OS(O)R^{1a}, wherein R^{1a} is as defined herein. In certain embodiments, R⁶ is —OS(O)₂R^{1a}, wherein R^{1a} is as defined herein. In certain embodiments, R⁶ is —OS(O)NR^{1b}R^{1c}, wherein R^{1b} and R^{1c} are each as defined herein. In certain embodiments, R⁶ is —OS(O)₂NR^{1b}R^{1c}, wherein R^{1b} and R^{1c} are each as defined herein. In certain embodiments, R⁶ is —NR^{1b}R^{1c}, wherein R^{1b} and R^{1c} are each as defined herein. In certain embodiments, R⁶ is —NR^{1a}C(O)R^{1d}, wherein R^{1a} and R^{1d} are each as defined herein. In certain embodiments, R⁶ is —NR^{1a}C(O)OR^{1d}, wherein R^{1a} and R^{1d} are each as defined herein. In certain embodiments, R⁶ is —NR^{1a}C(O)NR^{1b}R^{1c}, wherein R^{1a}, R^{1b}, and R^{1c} are each as defined herein. In certain embodiments, R⁶ is —NR^{1a}C(O)SR^{1d}, wherein R^{1a} and R^{1d} are each as defined herein. In certain embodiments, R⁶ is —NR^{1a}C(NR^{1d})NR^{1b}R^{1c}, wherein R^{1a}, R^{1b}, R^{1c}, and R^{1d} are each as defined herein. In certain embodiments, R⁶ is —NR^{1a}C(S)R^{1d}, wherein R^{1a} and R^{1d} are each as defined herein. In certain embodiments, R⁶ is —NR^{1a}C(S)OR^{1d}, wherein R^{1a} and R^{1d} are each as defined herein. In certain embodiments, R⁶ is —NR^{1a}C(S)NR^{1b}R^{1c}, wherein R^{1a}, R^{1b}, and R^{1c} are each as defined herein. In certain embodiments, R⁶ is —NR^{1a}S(O)R^{1d}, wherein R^{1a} and R^{1d} are each as defined herein. In certain embodiments, R⁶ is —NR^{1a}S(O)₂R^{1d}, wherein R^{1a} and R^{1d} are each as defined herein. In certain embodiments, R⁶ is —NR^{1a}S(O)NR^{1b}R^{1c}, wherein R^{1a}, R^{1b}, and R^{1c} are each as defined herein. In certain embodiments, R⁶ is —NR^{1a}S(O)₂NR^{1b}R^{1c}, wherein R^{1a}, R^{1b}, and R^{1c} are each as defined herein. In certain embodiments, R⁶ is —SR^{1a}, wherein R^{1a} is as defined herein. In certain embodiments, R⁶ is —S(O)R^{1a}, wherein R^{1a} is as defined herein. In certain embodiments, R⁶ is —S(O)₂R^{1a}, wherein R^{1a} is as defined herein. In certain embodiments, R⁶ is —S(O)NR^{1b}R^{1c}, wherein R^{1b} and R^{1c} are each as defined herein. In certain embodiments, R⁶ is —S(O)₂NR^{1b}R^{1c}, wherein R^{1b} and R^{1c} are each as defined herein.

In certain embodiments, R⁷ is C₂₋₆ alkyl, optionally substituted with one or more substituents Q. In certain embodiments, R⁷ is C₁₋₆ heteroalkyl, optionally substituted with one or more substituents Q. In certain embodiments, R⁷ is C₂₋₆ alkenyl, optionally substituted with one or more substituents Q. In certain embodiments, R⁷ is C₂₋₆ alkynyl, optionally substituted with one or more substituents Q. In certain embodiments, R⁷ is C₃₋₁₀ cycloalkyl, optionally substituted with one or more substituents Q. In certain embodiments, R⁷ is C₆₋₁₄ aryl, optionally substituted with one or more substituents Q. In certain embodiments, R⁷ is C₇₋₁₅ aralkyl, optionally substituted with one or more substituents Q. In certain embodiments, R⁷ is heteroaryl, optionally substituted with one or more substituents Q. In certain embodiments, R⁷ is heterocyclyl, optionally substituted with one or more substituents Q. In certain embodiments, R⁷ is —OR^{7a}, wherein R^{7a} is as defined herein. In certain embodiments, R⁷ is —NR^{7b}R^{7c}, wherein R^{7b} and R^{7c} are each as defined herein. In certain embodiments, R⁷ is —NR^{7d}C(O)R^{7e}, wherein R^{7d} and R^{7e} are each as defined herein. In certain embodiments, R⁷ is —NR^{7d}C(O)OR^{7e}, wherein R^{7d} and R^{7c} are each as defined herein. In certain embodiments, R⁷ is —NR^{7d}C(O)NR^{7b}R^{7c}, wherein R^{7b}, R^{7c}, and R^{7d} are each as defined herein.

In certain embodiments, R^{6a} is deuterium. In certain embodiments, R^{6a} is cyano. In certain embodiments, R^{6a} is halo. In certain embodiments, R^{6a} is fluoro. In certain embodiments, R^{6a} is nitro. In certain embodiments, R^{6a} is C₁₋₆ alkyl, optionally substituted with one or more substituents Q. In certain embodiments, R^{6a} is C₁₋₆ heteroalkyl, optionally substituted with one or more substituents Q. In certain embodiments, R^{6a} is C₂₋₆ alkenyl, optionally substituted with one or more substituents Q. In certain embodiments, R^{6a} is C₂₋₆ alkynyl, optionally substituted with one or more substituents Q. In certain embodiments, R^{6a} is C₃₋₁₀ cycloalkyl, optionally substituted with one or more substituents Q. In certain embodiments, R^{6a} is C₆₋₁₄ aryl, optionally substituted with one or more substituents Q. In certain embodiments, R^{6a} is C₇₋₁₅ aralkyl, optionally substituted with one or more substituents Q. In certain embodiments, R^{6a} is heteroaryl, optionally substituted with one or more substituents Q. In certain embodiments, R^{6a} is heterocyclyl, optionally substituted with one or more substituents Q.

In certain embodiments, R^{6a} is —C(O)R^{1a}, wherein R^{1a} is as defined herein. In certain embodiments, R^{6a} is —C(O)OR^{1a}, wherein R^{1a} is as defined herein. In certain embodiments, R^{6a} is —C(O)NR^{1b}R^{1c}, wherein R^{1b} and R^{1c} are each as defined herein. In certain embodiments, R^{6a} is —C(O)SR^{1a}, wherein R^{1a} is as defined herein. In certain embodiments, R^{6a} is —C(NR^{1a})NR^{1b}R^{1c}, wherein R^{1a}, R^{1b}, and R^{1c} are each as defined herein. In certain embodiments, R^{6a} is —C(S)R^{1a}, wherein R^{1a} is as defined herein. In certain embodiments, R^{6a} is —C(S)OR^{1a}, wherein R^{1a} is as defined herein. In certain embodiments, R^{6a} is —C(S)NR^{1b}R^{1e}, wherein R^{1b} and R^{1c} are each as defined herein. In certain embodiments, R^{6a} is —OR^{1a}, wherein R^{1a} is as defined herein. In certain embodiments, R^{6a} is —OC(O)R^{1a}, wherein R^{1a} is as defined herein. In certain embodiments, R^{6a} is —OC(O)OR^{1a}, wherein R^{1a} is as defined herein. In certain embodiments, R^{6a} is —OC(O)NR^{1b}R^{1e}, wherein R^{1b} and R^{1c} are each as defined herein. In certain embodiments, R^{6a} is —OC(S)R^{1a}, wherein R^{1a} is as defined herein. In certain embodiments, R^{6a} is —OC(NR^{1a})NR^{1b}R^{1c}, wherein R^{1a}, R^{1b}, and R^{1c} are each as defined herein. In certain embodiments, R^{6a} is —OC(S)R^{1a}, wherein R^{1a} is as defined herein. In certain embodiments, R^{6a} is —OC(S)OR^{1a}, wherein R^{1a} is as defined herein. In certain embodiments, R^{6a} is —OC(S)NR^{1b}R^{1c}, wherein R^{1b} and R^{1c} are each as defined herein. In certain embodiments, R^{6a} is —OS(O)R^{1a}, wherein R^{1a} is as defined herein. In certain embodiments, R^{6a} is —OS(O)₂R^{1a}, wherein R^{1a} is as defined herein. In certain embodiments, R^{6a} is —OS(O)NR^{1b}R^{1c}, wherein R^{1b} and R^{1c} are each as defined herein. In certain embodiments, R^{6a} is —OS(O)₂NR^{1b}R^{1c}, wherein R^{1b} and R^{1c} are each as defined herein. In certain embodiments, R^{6a} is —NR^{1b}R^{1c}, wherein R^{1b} and R^{1c} are each as defined herein. In certain embodiments, R^{6a} is —NR^{1a}C(O)R^{1d}, wherein R^{1a} and R^{1d} are each as defined herein. In certain embodiments, R^{6a} is —NR^{1a}C(O)OR^{1d}, wherein R^{1a} and R^{1d} are each as defined herein. In certain embodiments, R^{6a} is —NR^{1a}C(O)NR^{1b}R^{1c}, wherein R^{1a}, R^{1b}, and R^{1c} are each as defined herein. In certain embodiments, R^{6a} is —NR^{1a}C(O)SR^{1d}, wherein R^{1a} and R^{1d} are each as defined herein. In certain embodiments, R^{6a} is —NR^{1a}C(NR^{1d})NR^{1b}R^{1c}, wherein R^{1a}, R^{1b}, R^{1c}, and R^{1d} are each as defined herein. In certain embodiments, R^{6a} is —NR^{1a}C(S)R^{1d}, wherein R^{1a} and R^{1d} are each as defined herein. In certain embodiments, R^{6a} is —NR^{1a}C(S)OR^{1d}, wherein R^{1a} and R^{1d} are each as defined herein. In certain embodiments, R^{6a} is —NR^{1a}C(S)NR^{1b}R^{1c}, wherein R^{1a}, R^{1b}, and R^{1c} are each as defined herein. In certain embodiments, R^{6a} is —NR^{1a}S(O)R^{1d}, wherein R^{1a} and R^{1d} are each as defined herein. In certain embodiments, R^{6a} is —NR^{1a}S(O)₂R^{1d}, wherein R^{1a} and R^{1d} are each as defined herein. In certain embodiments, R^{6a} is —NR^{1a}S(O)NR^{1b}R^{1c}, wherein R^{1a}, R^{1b}, and R^{1c} are each as defined herein. In certain embodiments, R^{6a} is —NR^{1a}S(O)₂NR^{1b}R^{1c}, wherein R^{1a}, R^{1b}, and R^{1c} are each as defined herein. In certain embodiments, R^{6a} is —SR^{1a}, wherein R^{1a} is as defined herein. In certain embodiments, R^{6a} is —S(O)R^{1a}, wherein R^{1a} is as defined herein. In certain embodiments, R^{6a} is —S(O)₂R^{1a}, wherein R^{1a} is as defined herein. In certain embodiments, R^{6a} is —S(O)NR^{1b}R^{1c}, wherein R^{1b} and R^{1c} are each as defined herein. In certain embodiments, R^{6a} is —S(O)₂NR^{1b}R^{1c}, wherein R^{1b} and R^{1c} are each as defined herein.

In certain embodiments, R^{7a} is C₁₋₆ alkyl, optionally substituted with one or more substituents Q. In certain embodiments, R^{7a} is methyl, optionally substituted with one or more substituents Q. In certain embodiments, R^{7a} is methyl, optionally substituted with —OC(O)R^{1a}, wherein R^{1a} is as defined herein. In certain embodiments, R^{7a} is pivaloyloxymethyl, benzoyloxymethyl, or isopropyl. In certain embodiments, R^{7a} is C₁₋₆ heteroalkyl, optionally substituted with one or more substituents Q. In certain embodiments, R^{7a} is C₂₋₆ alkenyl, optionally substituted with one or more substituents Q. In certain embodiments, R^{7a} is C₂₋₆ alkynyl, optionally substituted with one or more substituents Q. In certain embodiments, R^{7a} is monocyclic C₃₋₁₀ cycloalkyl, optionally substituted with one or more substituents Q. In certain embodiments, R^{7a} is cyclopentyl. In certain embodiments, R^{7a} is bicyclic C₃₋₁₀ cycloalkyl, optionally substituted with one or more substituents Q. In certain embodiments, R^{7a} is C₆₋₁₄ aryl, optionally substituted with one or more substituents Q. In certain embodiments, R^{7a} is phenyl, optionally substituted with one or more substituents Q. In certain embodiments, R^{7a} is 4-methylphenyl. In certain embodiments, R^{7a} is C₇₋₁₅ aralkyl, optionally substituted with one or more substituents Q. In certain embodiments, R^{7a} is heteroaryl, optionally substituted with one or more substituents Q. In certain embodiments, R^{7a} is heterocyclyl, optionally substituted with one or more substituents Q.

In certain embodiments, R^{7b} is hydrogen. In certain embodiments, R^{7b} is C₁₋₆ alkyl, optionally substituted with one or more substituents Q. In certain embodiments, R^{7b} is methyl, ethyl, propyl, or butyl, each optionally substituted with one or more substituents Q. In certain embodiments, R^{7b} is C₁₋₆ heteroalkyl, optionally substituted with one or more substituents Q. In certain embodiments, R^{7b} is C₂₋₆ alkenyl, optionally substituted with one or more substituents Q. In certain embodiments, R^{7b} is C₂₋₆ alkynyl, optionally substituted with one or more substituents Q. In certain embodiments, R^{7b} is C₃₋₁₀ cycloalkyl, optionally substituted with one or more substituents Q. In certain embodiments, R^{7b} is C₆₋₁₄ aryl, optionally substituted with one or more substituents Q. In certain embodiments, R^{7b} is C₇₋₁₅ aralkyl, optionally substituted with one or more substituents Q. In certain embodiments, R^{7b} is heteroaryl, optionally substituted with one or more substituents Q. In certain embodiments, R^{7b} is heterocyclyl, optionally substituted with one or more substituents Q. In certain embodiments, R^{7b} is hydrogen, methyl, hydroxycarbonylmethyl, ethyl, 2-hydroxyethyl, 2-methoxyethyl, propyl, isopropyl, or 1-(hydroxycarbonyl)-2-methylpropyl.

In certain embodiments, R^{7c} is hydrogen. In certain embodiments, R^{7c} is C₁₋₆ alkyl, optionally substituted with one or more substituents Q. In certain embodiments, R^{7c} is methyl, ethyl, propyl, or butyl, each optionally substituted with one or more substituents Q. In certain embodiments, R^{7c} is C₁₋₆ heteroalkyl, optionally substituted with one or more substituents Q. In certain embodiments, R^{7c} is C₂₋₆ alkenyl, optionally substituted with one or more substituents Q. In certain embodiments, R^{7c} is C₂₋₆ alkynyl, optionally substituted with one or more substituents Q. In certain embodiments, R^{7c} is C₃₋₁₀ cycloalkyl, optionally substituted with one or more substituents Q. In certain embodiments, R^{7c} is C₆₋₁₄ aryl, optionally substituted with one or more substituents Q. In certain embodiments, R^{7c} is C₇₋₁₅ aralkyl, optionally substituted with one or more substituents Q. In certain embodiments, R^{7c} is heteroaryl, optionally substituted with one or more substituents Q. In certain embodiments, R^{7c} is heterocyclyl, optionally substituted with one or more substituents Q. In certain embodiments, R^{7c} is hydrogen, methyl, hydroxycarbonylmethyl, ethyl, 2-hydroxyethyl, 2-methoxyethyl, propyl, isopropyl, or 1-(hydroxycarbonyl)-2-methylpropyl.

In certain embodiments, R^{7b} and R^{7c} together with the N atom to which they are attached form heteroaryl, optionally substituted with one or more substituents Q. In certain embodiments, R^{7b} and R^{7c} together with the N atom to which they are attached form monocyclic heteroaryl, optionally substituted with one, two, or three substituents Q. In certain embodiments, R^{7b} and R^{7c} together with the N atom to which they are attached form 5- or 6-membered heteroaryl, each optionally substituted with one, two, or three substituents Q. In certain embodiments, R^{7b} and R^{7c} together with the N atom to which they are attached form 5-membered heteroaryl, optionally substituted with one, two, or three substituents Q. In certain embodiments, R^{7b} and R^{7c} together with the N atom to which they are attached form 6-membered heteroaryl, optionally substituted with one, two, or three substituents Q. In certain embodiments, R^{7b} and R^{7c} together with the N atom to which they are attached form bicyclic heteroaryl, optionally substituted with one, two, or three substituents Q. In certain embodiments, R^{7b} and R^{7c} together with the N atom to which they are attached form 5,5-, 5,6-, or 6,6-fused heteroaryl, each optionally substituted with one, two, or three substituents Q. In certain embodiments, R^{7b} and R^{7c} together with the N atom to which they are attached form imidazol-1-yl or 2-aminoimidazol-1-yl.

In certain embodiments, R^{7b} and R^{7c} together with the N atom to which they are attached form heterocyclyl, optionally substituted with one or more substituents Q. In certain embodiments, R^{7b} and R^{7c} together with the N atom to which they are attached form monocyclic heterocyclyl, optionally substituted with one, two, or three substituents Q. In certain embodiments, R^{7b} and R^{7c} together with the N atom to which they are attached form 3-, 4-, 5-, 6-, or 7-membered heterocyclyl, each optionally substituted with one, two, or three substituents Q. In certain embodiments, R^{7b} and R^{7c} together with the N atom to which they are attached form 5- or 6-membered heterocyclyl, each optionally substituted with one, two, or three substituents Q. In certain embodiments, R^{7b} and R^{7c} together with the N atom to which they are attached form 5-membered heterocyclyl, optionally substituted with one, two, or three substituents Q. In certain embodiments, R^{7b} and R^{7c} together with the N atom to which they are attached form 6-membered heterocyclyl, optionally substituted with one, two, or three substituents Q. In certain embodiments, R^{7b} and R^{7c} together with the N atom to which they are attached form bicyclic heterocyclyl, optionally substituted with one, two, or three substituents Q. In certain embodiments, R^{7b} and R^{7c} together with the N atom to which they are attached form bridged, fused, or spiro heterocyclyl, each optionally substituted with one, two, or three substituents Q. In certain embodiments, R^{7b} and R^{7c} together with the N atom to which they are attached form 3-hydroxyazetidin-1-yl, 2-oxo-5-(hydroxymethyl)oxazolidin-3-yl, 2-(hydroxymethyl)piperidin-1-yl, or morpholin-4-yl.

In certain embodiments, R^{7d} is hydrogen. In certain embodiments, R^{7d} is C₁₋₆ alkyl, optionally substituted with one or more substituents Q. In certain embodiments, R^{7d} is C₁₋₆ heteroalkyl, optionally substituted with one or more substituents Q. In certain embodiments, R^{7d} is C₂₋₆ alkenyl, optionally substituted with one or more substituents Q. In certain embodiments, R^{7d} is C₂₋₆ alkynyl, optionally substituted with one or more substituents Q. In certain embodiments, R^{7d} is C₃₋₁₀ cycloalkyl, optionally substituted with one or more substituents Q. In certain embodiments, R^{7d} is C₆₋₁₄ aryl, optionally substituted with one or more substituents Q. In certain embodiments, R^{7d} is C₇₋₁₅ aralkyl, optionally substituted with one or more substituents Q. In certain embodiments, R^{7d} is heteroaryl, optionally substituted with one or more substituents Q. In certain embodiments, R^{7d} is heterocyclyl, optionally substituted with one or more substituents Q.

In certain embodiments, R^{7e} is hydrogen. In certain embodiments, R^{7e} is C₁₋₆ alkyl, optionally substituted with one or more substituents Q. In certain embodiments, R^{7e} is methyl, ethyl, propyl, or butyl, each optionally substituted with one, two, or three substituents Q. In certain embodiments, R^{7e} is C₁₋₆ heteroalkyl, optionally substituted with one or more substituents Q. In certain embodiments, R^{7e} is C₂₋₆ alkenyl, optionally substituted with one or more substituents Q. In certain embodiments, R^{7e} is C₂₋₆ alkynyl, optionally substituted with one or more substituents Q. In certain embodiments, R^{7e} is C₃₋₁₀ cycloalkyl, optionally substituted with one or more substituents Q. In certain embodiments, R^{7e} is monocyclic C₃₋₁₀ cycloalkyl, optionally substituted with one, two, or three substituents Q. In certain embodiments, R^{7c} is cyclopropyl, cyclobutyl, cyclopentyl, or cyclohexyl, each optionally substituted with one, two, or three substituents Q. In certain embodiments, R^{7c} is C₆₋₁₄ aryl, optionally substituted with one or more substituents Q. In certain embodiments, R^{7e} is C₇₋₁₅ aralkyl, optionally substituted with one or more substituents Q. In certain embodiments, R^{7c} is heteroaryl, optionally substituted with one or more substituents Q. In certain embodiments, R^{7e} is heterocyclyl, optionally substituted with one or more substituents Q. In certain embodiments, R^{7c} is monocyclic heterocyclyl, optionally substituted with one, two, or three substituents Q. In certain embodiments, R^{7e} is 3-, 4-, 5-, 6-, or 7-membered heterocyclyl, each optionally substituted with one, two, or three substituents Q. In certain embodiments, R^{7e} is 5- or 6-membered heterocyclyl, each optionally substituted with one, two, or three substituents Q. In certain embodiments, R^{7c} is 5-membered heterocyclyl, optionally substituted with one, two, or three substituents Q. In certain embodiments, R^{7c} is 6-membered heterocyclyl, optionally substituted with one, two, or three substituents Q. In certain embodiments, R^{7e} is pyrrolidinyl, tetrahydrofuryl, or tetrahydropyranyl, each optionally substituted with one, two, or three substituents Q. In certain embodiments, R^{7e} is bicyclic heterocyclyl, optionally substituted with one, two, or three substituents Q. In certain embodiments, R^{7c} is bridged, fused, or spiro heterocyclyl, each optionally substituted with one, two, or three substituents Q.

In certain embodiments, R^{7e} is C₁₋₆ alkyl, optionally substituted with one, two, or three substituents, wherein each substituent is independently —C(O)OR^{1a}, —OR^{1a}, —OC(O)R^{1a}, —NR^{1b}R^{1c}, or —NR^{1a}C(O)R^{1d}; and wherein each R^{1a}, R^{1b}, R^{1c}, and R^{1d} is as defined herein. In certain embodiments, R^{7e} is C₁₋₆ alkyl substituted with —C(O)OR^{1a}, wherein R^{1a} is as defined herein. In certain embodiments, R^{7c} is C₁₋₆ alkyl substituted with —OR^{1a}, wherein R^{1a} is as defined herein. In certain embodiments, R^{e} is C₁₋₆ alkyl substituted with —OC(O)R^{1a}, wherein R^{1a} is as defined herein. In certain embodiments, R^{7c} is C₁₋₆ alkyl substituted with —NR^{1b}R^{1c}, wherein R^{1b} and R^{1c} are each as defined herein. In certain embodiments, R^{7e} is C₁₋₆ alkyl substituted with —NR^{1a}C(O)R^{1d}, wherein R^{1a} and R^{1d} are each as defined herein.

In certain embodiments, R^{e} is C₁₋₆ alkyl substituted with aminoacylamino. In certain embodiments, R^{7e} is C₁₋₆ alkyl substituted with α-aminoacylamino or β-aminoacylamino. In certain embodiments, R^{7e} is C₁₋₆ alkyl substituted with α-aminoacylamino, which is aminoacetamido, 2-aminopropanamido, 2-amino-3-phenylpropanamido, 2-amino-3-(4-hydroxy-phenyl)propanamido, 2-amino-3-(imidazol-4-yl)propanamido, 2-amino-3-(indol-3-yl)propanamido, 2-amino-3-(hydroxycarbonyl)propanamido, 2-amino-3-(aminocarbonyl)propanamido, 2-amino-3-hydroxypropanamido, 2-amino-3-mercaptopropanamido, 2-amino-3-methylbutan-amido, 2-amino-3-hydroxybutanamido, 2-amino-4-(methylthio)butanamido, 2-amino-4-hydroxycarbonylbutanamido, 2-amino-4-(aminocarbonyl)butanamido, 2-amino-3-methylpentanamido, 2-amino-4-methylpentanamido, 2,6-diaminohexanamido, 2-amino-5-guanidinopentamido, or pyrrolin-2-ylcarboxamido. In certain embodiments, R^{7e} is C₁₋₆ alkyl substituted with α-aminoacylamino, which is aminoacetamido, (R)-2-aminopropanamido, (R)-2-amino-3-phenylpropanamido, (R)-2-amino-3-(4-hydroxyphenyl)propanamido, (R)-2-amino-3-(imidazol-4-yl)propanamido, (R)-2-amino-3-(indol-3-yl)propanamido, (R)-2-amino-3-(hydroxycarbonyl)-propanamido, (R)-2-amino-3-(aminocarbonyl)propanamido, (R)-2-amino-3-hydroxypropanamido, (R)-2-amino-3-mercaptopropanamido, (R)-2-amino-3-methylbutanamido, (R)-2-amino-3-hydroxybutanamido, (R)-2-amino-4-(methylthio)butanamido, (R)-2-amino-4-hydroxycarbonylbutan-amido, (R)-2-amino-4-(aminocarbonyl)butanamido, (R)-2-amino-3-methylpentanamido, (R)-2-amino-4-methylpentanamido, (R)-2,6-diaminohexanamido, (R)-2-amino-5-guanidinopentamido, or (R)-pyrrolin-2-ylcarboxamido. In certain embodiments, R^{7c} is C₁_alkyl substituted with α-aminoacylamino, which is aminoacetamido, (S)-2-aminopropanamido, (S)-2-amino-3-phenylpropanamido, (S)-2-amino-3-(4-hydroxyphenyl)propanamido, (S)-2-amino-3-(imidazol-4-yl)propanamido, (S)-2-amino-3-(indol-3-yl)propanamido, (S)-2-amino-3-(hydroxycarbonyl)-propanamido, (S)-2-amino-3-(aminocarbonyl)propanamido, (S)-2-amino-3-hydroxypropanamido, (S)-2-amino-3-mercaptopropanamido, (S)-2-amino-3-methylbutanamido, (S)-2-amino-3-hydroxybutanamido, (S)-2-amino-4-(methylthio)butanamido, (S)-2-amino-4-hydroxycarbonyl-butanamido, (S)-2-amino-4-(aminocarbonyl)butanamido, (S)-2-amino-3-methylpentanamido, (S)-2-amino-4-methylpentanamido, (S)-2,6-diaminohexanamido, (S)-2-amino-5-guanidinopentamido, or (S)-pyrrolin-2-ylcarboxamido.

In certain embodiments, R^{7e} is ethyl substituted with aminoacylamino. In certain embodiments, R^{7e} is ethyl substituted at the 2-position with α-aminoacylamino or 3-aminoacylamino. In certain embodiments, R^{7c} is and R^{7c} is ethyl substituted at the 2-position with α-aminoacylamino, which is aminoacetamido, 2-aminopropanamido, 2-amino-3-phenylpropanamido, 2-amino-3-(4-hydroxyphenyl)propanamido, 2-amino-3-(imidazol-4-yl)propanamido, 2-amino-3-(indol-3-yl)propanamido, 2-amino-3-(hydroxycarbonyl)propanamido, 2-amino-3-(aminocarbonyl)propanamido, 2-amino-3-hydroxypropanamido, 2-amino-3-mercaptopropanamido, 2-amino-3-methylbutanamido, 2-amino-3-hydroxybutanamido, 2-amino-4-(methylthio)butanamido, 2-amino-4-hydroxycarbonylbutanamido, 2-amino-4-(aminocarbonyl)-butanamido, 2-amino-3-methylpentanamido, 2-amino-4-methylpentanamido, 2,6-diaminohexanamido, 2-amino-5-guanidinopentamido, or pyrrolin-2-ylcarboxamido. In certain embodiments, R^{7c} is ethyl substituted at the 2-position with α-aminoacylamino, which is aminoacetamido, (R)-2-aminopropanamido, (R)-2-amino-3-phenylpropanamido, (R)-2-amino-3-(4-hydroxyphenyl)-propanamido, (R)-2-amino-3-(imidazol-4-yl)propanamido, (R)-2-amino-3-(indol-3-yl)propanamido, (R)-2-amino-3-(hydroxycarbonyl)propanamido, (R)-2-amino-3-(aminocarbonyl)propanamido, (R)-2-amino-3-hydroxypropanamido, (R)-2-amino-3-mercaptopropanamido, (R)-2-amino-3-methylbutanamido, (R)-2-amino-3-hydroxybutanamido, (R)-2-amino-4-(methylthio)-butanamido, (R)-2-amino-4-hydroxycarbonylbutanamido, (R)-2-amino-4-(aminocarbonyl)butanamido, (R)-2-amino-3-methylpentanamido, (R)-2-amino-4-methylpentanamido, (R)-2,6-diaminohexanamido, (R)-2-amino-5-guanidinopentamido, or (R)-pyrrolin-2-ylcarboxamido. In certain embodiments, R^{7e} is ethyl substituted at the 2-position with α-aminoacylamino, which is aminoacetamido, (S)-2-aminopropanamido, (S)-2-amino-3-phenylpropanamido, (S)-2-amino-3-(4-hydroxyphenyl)propanamido, (S)-2-amino-3-(imidazol-4-yl)propanamido, (S)-2-amino-3-(indol-3-yl)propanamido, (S)-2-amino-3-(hydroxycarbonyl)propanamido, (S)-2-amino-3-(amino-carbonyl)propanamido, (S)-2-amino-3-hydroxypropanamido, (S)-2-amino-3-mercaptopropanamido, (S)-2-amino-3-methylbutanamido, (S)-2-amino-3-hydroxybutanamido, (S)-2-amino-4-(methylthio)butanamido, (S)-2-amino-4-hydroxycarbonylbutanamido, (S)-2-amino-4-(amino-carbonyl)butanamido, (S)-2-amino-3-methylpentanamido, (S)-2-amino-4-methylpentanamido, (S)-2,6-diaminohexanamido, (S)-2-amino-5-guanidinopentamido, or (S)-pyrrolin-2-ylcarboxamido.

In certain embodiments, R^{7e} is methyl, ethyl, propyl, or butyl, each optionally substituted with one, two, or three substituents, wherein each substituent is independently imidazol-4-yl, hydroxycarbonyl, ethoxycarbonyl, hydroxyl, methoxy, acetoxy, amino, acetamido, aminoacetamido, 2-aminopropanamido, 2-amino-3-hydroxypropanamido, 2-amino-3-methylbutanamido, 2-amino-4-(methylthio)butanamido, 2-amino-4-methylpentanamido, or pyrrolidin-2-ylamido.

In certain embodiments, R^{7c} is methyl, hydroxycarbonylmethyl, ethoxycarbonylmethyl, aminomethyl, ethyl, 2-hydroxyethyl, 2-methoxyethyl, 2-acetoxyethyl, 1-aminoethyl, 1-amino-2-methylpropyl, 1,5-diaminopentyl, 2-aminoethyl, 1-amino-2-(imidazol-4-yl)ethyl, 2-acetamidoethyl, 2-(2-aminoacetamido)ethyl, 2-(2-aminopropanamido)ethyl, 2-(2-amino-3-hydroxypropanamido)ethyl, 2-(2-amino-3-methylbutanamido)ethyl, 2-(2-amino-4-(methylthio)-butanamido)ethyl, 2-(2-amino-4-methylpentanamido)ethyl, 2-(pyrrolidin-2-ylamido)ethyl, isopropyl, tert-butyl, cyclopropyl, cyclobutyl, pyrrolidin-2-yl, tetrahydrofuran-2-yl, or tetrahydropyran-4-yl. In certain embodiments, R^{7e} is methyl, hydroxycarbonylmethyl, ethoxycarbonylmethyl, aminomethyl, ethyl, 2-hydroxyethyl, 2-methoxyethyl, 2-acetoxyethyl, 1-aminoethyl, 1-amino-2-methylpropyl, 1,5-diaminopentyl, 2-aminoethyl, 1-amino-2-(imidazol-4-yl)-ethyl, 2-acetamidoethyl, 2-(2-aminoacetamido)ethyl, (R)-2-(2-aminopropanamido)ethyl, (S)-2-(2-amino-3-hydroxypropanamido)ethyl, (S)-2-(2-amino-3-methylbutanamido)ethyl, (S)-2-(2-amino-4-(methylthio)butanamido)ethyl, (S)-2-(2-amino-4-methylpentanamido)ethyl, (S)-2-(pyrrolidin-2-ylamido)ethyl, isopropyl, tert-butyl, cyclopropyl, cyclobutyl, pyrrolidin-2-yl, tetrahydrofuran-2-yl, or tetrahydropyran-4-yl.

In certain embodiments, m is an integer of 0. In certain embodiments, m is an integer of 1. In certain embodiments, m is an integer of 2. In certain embodiments, m is an integer of 3.

In certain embodiments, n is an integer of 0. In certain embodiments, n is an integer of 1. In certain embodiments, n is an integer of 2. In certain embodiments, n is an integer of 3. In certain embodiments, n is an integer of 4.

In one embodiment, provided herein is:
2-hydroxyethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A1;
tert-butyl (S)-((4-(6-((6-acetyl-8-cyclopentyl-5-methyl-7-oxo-7,8-dihydropyrido[2,3-d]pyrimidin-2-yl)amino)pyridin-3-yl)-3-methylpiperazin-1-yl)sulfonyl)carbamate A2;
(S)-4-(6-((6-acetyl-8-cyclopentyl-5-methyl-7-oxo-7,8-dihydropyrido[2,3-d]-pyrimidin-2-yl)amino)pyridin-3-yl)-3-methylpiperazine-1-sulfonamide A3;
tert-butyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A4;
4-(6-((6-acetyl-8-cyclopentyl-5-methyl-7-oxo-7,8-dihydropyrido[2,3-d]pyrimidin-2-yl)amino)pyridin-3-yl)-piperazine-1-sulfonamide A5;
(S)—N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-aminopropanamide A6;
(R)-6-acetyl-8-cyclopentyl-2-((5-(4-((2-(hydroxymethyl)piperidin-1-yl)sulfonyl)-piperazin-1-yl)pyridin-2-yl)amino)-5-methylpyrido[2,3-d]pyrimidin-7(8H)-one A7;
2-methoxyethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A8;
2-((((4-(6-((6-acetyl-8-cyclopentyl-5-methyl-7-oxo-7,8-dihydropyrido[2,3-cd]-pyrimidin-2-yl)amino)pyridin-3-yl)piperazin-1-yl)sulfonyl)carbamoyl)oxy)ethyl acetate A9;
ethyl 2-((((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamoyl)oxy)acetate A10;
2-((((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamoyl)oxy)acetic acid A11;
3-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-5-(hydroxymethyl)oxazolidin-2-one A12;
2-aminoethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A13;
(S)-2-(2-amino-3-methylbutanamido)ethyl ((4-(6-((6-acetyl-8-cyclopentyl-5-methyl-7-oxo-7,8-dihydropyrido[2,3-]pyrimidin-2-yl)amino)pyridin-3-yl)piperazin-1-yl)sulfonyl)carbamate A14;
2-acetamidoethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A15;
(R)-2-(2-aminopropanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)-carbamate A16;
(S)-2-(2-amino-4-methylpentanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)-sulfonyl)carbamate A17;
(S)-2-(2-amino-3-hydroxypropanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)-sulfonyl)carbamate A18;
(S)-2-(2-amino-4-(methylthio)butanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)-sulfonyl)carbamate A19;
2-(2-aminoacetamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A20;
(R)-2-(pyrrolidine-2-carboxamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)-carbamate A21;
tetrahydro-2H-pyran-4-yl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A22;
cyclobutyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A23;
cyclopropyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A24;
ethyl ((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamate A25;
methyl ((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamate A26;
isopropyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A27;
(S)—N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-aminobutanamide A28;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-aminoacetamide A29;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-amino-3-methylbutanamide A30;
(S)—N-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)-2-amino-3-(1H-imidazol-4-yl)-propanamide A31;
(S)—N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2,6-diaminohexanamide A32;
(R)—N-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)pyrrolidine-2-carboxamide A33;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)acetamide A34;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)propionamide A35;
N-((4-(6-((6-acetyl-8-cyclopentyl-5-methyl-7-oxo-7,8-dihydropyrido[2,3-cd]-pyrimidin-2-yl)amino)pyridin-3-yl)piperazin-1-yl)sulfonyl)isobutyramide A36;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)butyramide A37;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)cyclopropanecarboxamide A38;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)tetrahydrofuran-2-carboxamide A39;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)tetrahydro-2H-pyran-4-carboxamide A40;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)cyclobutanecarboxamide A41;
4-(6-((6-acetyl-8-cyclopentyl-5-methyl-7-oxo-7,8-dihydropyrido[2,3-d]-pyrimidin-2-yl)amino)pyridin-3-yl)-N-(2-hydroxyethyl)piperazine-1-sulfonamide A42;
4-(6-((6-acetyl-8-cyclopentyl-5-methyl-7-oxo-7,8-dihydropyrido[2,3-d]-pyrimidin-2-yl)amino)pyridin-3-yl)-N-(2-methoxyethyl)piperazine-1-sulfonamide A43;
((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)glycine A44;
((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-L-valine A45;
4-(6-((6-acetyl-8-cyclopentyl-5-methyl-7-oxo-7,8-dihydropyrido[2,3-d]-pyrimidin-2-yl)amino)pyridin-3-yl)-N-methylpiperazine-1-sulfonamide A46;
4-(6-((6-acetyl-8-cyclopentyl-5-methyl-7-oxo-7,8-dihydropyrido[2,3-d]-pyrimidin-2-yl)amino)pyridin-3-yl)-N-ethylpiperazine-1-sulfonamide A47;
4-(6-((6-acetyl-8-cyclopentyl-5-methyl-7-oxo-7,8-dihydropyrido[2,3-d]-pyrimidin-2-yl)amino)pyridin-3-yl)-N-isopropylpiperazine-1-sulfonamide A48;
4-(6-((6-acetyl-8-cyclopentyl-5-methyl-7-oxo-7,8-dihydropyrido[2,3-d]-pyrimidin-2-yl)amino)pyridin-3-yl)-N-propylpiperazine-1-sulfonamide A49;
4-(6-((6-acetyl-8-cyclopentyl-5-methyl-7-oxo-7,8-dihydropyrido[2,3-d]-pyrimidin-2-yl)amino)pyridin-3-yl)-N,N-dimethylpiperazine-1-sulfonamide A50;
6-acetyl-8-cyclopentyl-5-methyl-2-((5-(4-(morpholinosulfonyl)piperazin-1-yl)-pyridin-2-yl)amino)pyrido[2,3-d]pyrimidin-7(8H)-one A51;
6-acetyl-8-cyclopentyl-2-((5-(4-((3-hydroxyazetidin-1-yl)sulfonyl)piperazin-1-yl)pyridin-2-yl)amino)-5-methylpyrido[2,3-d]pyrimidin-7(8H)-one A52;
4-(6-((6-acetyl-8-cyclopentyl-5-methyl-7-oxo-7,8-dihydropyrido[2,3-d]-pyrimidin-2-yl)amino)pyridin-3-yl)-N,N-bis(2-hydroxyethyl)piperazine-1-sulfonamide A53;
2-hydroxyethyl (S)-((4-(6-((6-acetyl-8-cyclopentyl-5-methyl-7-oxo-7,8-dihydropyrido[2,3-d]pyrimidin-2-yl)amino)pyridin-3-yl)-3-methylpiperazin-1-yl)sulfonyl)carbamate A54;
2-((R)-2-amino-3-methylbutanamido)ethyl (((S)-4-(6-((6-acetyl-8-cyclopentyl-5-methyl-7-oxo-7,8-dihydropyrido[2,3-d]pyrimidin-2-yl)amino)pyridin-3-yl)-3-methylpiperazin-1-yl)sulfonyl)carbamate A55;
4-(6-((6-acetyl-8-cyclopentyl-5-methyl-7-oxo-7,8-dihydropyrido[2,3-d]-pyrimidin-2-yl)amino)pyridin-3-yl)-N-(isopropylcarbamoyl)piperazine-1-sulfonamide A56;
6-acetyl-2-((5-(4-((2-amino-1H-imidazol-1-yl)sulfonyl)piperazin-1-yl)pyridin-2-yl)amino)-8-cyclopentyl-5-methylpyrido[2,3-d]pyrimidin-7(8H)-one A57; or
2-((5-(4-((1H-imidazol-1-yl)sulfonyl)piperazin-1-yl)pyridin-2-yl)amino)-6-acetyl-8-cyclopentyl-5-methylpyrido[2,3-d]pyrimidin-7(8H)-one A58;
   or an enantiomer, a mixture of enantiomers, a diastereomer, a mixture of two or more diastereomers, a tautomer, or a mixture of two or more tautomers thereof, or a pharmaceutically acceptable salt, solvate, hydrate, or prodrug thereof.

In another embodiment, provided herein is a compound of:
(((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)oxy)methyl pivalate B1;
p-tolyl 4-(6-((6-acetyl-8-cyclopentyl-5-methyl-7-oxo-7,8-dihydropyrido[2,3-d]-pyrimidin-2-yl)amino)pyridin-3-yl)piperazine-1-sulfonate B2;
(((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)oxy)methyl benzoate B3; or
cyclopentyl 4-(6-((6-acetyl-8-cyclopentyl-5-methyl-7-oxo-7,8-dihydropyrido[2,3-d]pyrimidin-2-yl)amino)pyridin-3-yl)piperazine-1-sulfonate B4;
   or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof.

In yet another embodiment, provided herein is a compound of:
6-acetyl-8-cyclopentyl-2-[[5-(4-isopropylsulfonylpiperazin-1-yl)-2-pyridyl]-amino]-5-methylpyrido[2,3-d]pyrimidin-7-one C1; or
6-acetyl-8-cyclopentyl-2-((5-(4-(ethylsulfonyl)piperazin-1-yl)pyridin-2-yl)-amino)-5-methylpyrido[2,3-d]pyrimidin-7(8H)-one C2;
   or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof.

In certain embodiments, a compound provided herein is isolated or purified. In certain embodiments, a compound provided herein has a purity of at least about 90%, at least about 95%, at least about 98%, at least about 99%, or at least about 99.5% by weight. In certain embodiments, a compound provided herein has a purity of at least about 90% by weight. In certain embodiments, a compound provided herein has a purity of at least about 95% by weight. In certain embodiments, a compound provided herein has a purity of at least about 98% by weight. In certain embodiments, a compound provided herein has a purity of at least about 99% by weight. In certain embodiments, a compound provided herein has a purity of at least about 99.5% by weight.

The compounds provided herein are intended to encompass all possible stereoisomers unless a particular stereochemistry is specified. Where a compound provided herein contains an alkenyl group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contains an aromatic moiety. It follows that a single compound may exhibit more than one type of isomerism.

A compound provided herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for the compound that undergoes epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethyl-amino)ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound provided herein may also be provided as a prodrug, which is a functional derivative of the compound and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### Pharmaceutical Compositions

In one embodiment, provided herein is a pharmaceutical composition, comprising a compound provided herein, e.g., a compound of Formula (I), or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; and a pharmaceutically acceptable excipient.

The pharmaceutical composition provided herein can be formulated in various dosage forms, including, but not limited to, dosage forms for oral administration. The pharmaceutical composition can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd ed.; Rathbone et al., Eds.; Drugs and the Pharmaceutical Sciences 184; CRC Press: Boca Raton, FL, 2008.

The pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) (e.g., a compound provided herein) sufficient to produce the desired therapeutic effect, in association with the required pharmaceutical excipient(s). Examples of a unit-dosage form include, but are not limited to, individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial, bottle of tablets or capsules.

The pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

### A. Oral Administration

The pharmaceutical composition provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical composition can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500®); gelatin; sugars, such as sucrose, glucose, dextrose, molasses, and lactose; natural and synthetic gums, such as acacia, alginic acid, alginates, extract of Irish moss, Panwar gum, Ghatti gum, mucilage of isabgol husks, carboxymethylcellulose, methylcellulose, polyvinylpyrrolidone (PVP), VEEGUM®, larch arabinogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); and microcrystalline celluloses, such as AVICEL® PH-101, AVICEL® PH-103, AVICEL® PH-105, and AVICEL® RC-581. Suitable fillers include, but are not limited to, talc, calcium carbonate, microcrystalline cellulose, powdered cellulose, dextrates, kaolin, mannitol, silicic acid, sorbitol, starch, and pre-gelatinized starch. The amount of a binder or filler in the pharmaceutical composition provided herein varies upon the type of formulation, and is readily discernible to those of ordinary skill in the art. The binder or filler may be present from about 50 to about 99% by weight in the pharmaceutical composition provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical composition provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and VEEGUM® HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; and algins. The amount of a disintegrant in the pharmaceutical composition provided herein varies upon the type of formulation, and is readily discernible to those of ordinary skill in the art. The pharmaceutical composition provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, such as peanut oil, cottonseed oil, sunflower oil, sesame oil, olive oil, corn oil, and soybean oil; zinc stearate; ethyl oleate; ethyl laureate; agar; starch; lycopodium; and silica or silica gels, such as AEROSIL© 200 and CAB-O-SIL®. The amount of a lubricant in the pharmaceutical composition provided herein varies upon the type of formulation, and is readily discernible to those of ordinary skill in the art. The pharmaceutical compositions provided herein may contain about 0.1 to about 5% by weight of a lubricant.

Suitable glidants include, but are not limited to, colloidal silicon dioxide, CAB-O-SIL®, and asbestos-free talc. Suitable coloring agents include, but are not limited to, any of the approved, certified, water soluble FD&C dyes, and water insoluble FD&C dyes suspended on alumina hydrate, and color lakes. A color lake is a combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, VEEGUM®, acacia, sodium carboxymethylcellulose, hydroxypropyl methylcellulose, and polyvinylpyrrolidone. Suitable preservatives include, but are not limited to, glycerin, methyl and propylparaben, benzoic add, and sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical composition provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredient(s) from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from an active ingredient(s) in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical composition provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient(s). The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient(s).

The pharmaceutical composition provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing an active ingredient(s), and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These dosage forms can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical composition provided herein for oral administration can be also provided in the forms of liposomes, micelles, microspheres, or nanosystems. Micellar dosage forms can be prepared as described in U.S. Pat. No. 6,350,458.

The pharmaceutical composition provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the dosage forms described herein.

The pharmaceutical composition provided herein for oral administration can be formulated as immediate or modified release dosage forms, including delayed-, sustained, pulsed-, controlled, targeted-, and programmed-release forms.

### B. Modified Release

The pharmaceutical composition provided herein can be formulated as a modified release dosage form. As used herein, the term “modified release” refers to a dosage form in which the rate or place of release of an active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical composition in modified release dosage forms can be prepared using a variety of modified release devices and methods known to those skilled in the art, including, but not limited to, matrix-controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

### 1. Matrix Controlled Release Devices

The pharmaceutical composition provided herein in a modified release dosage form can be fabricated using a matrix-controlled release device known to those skilled in the art. See, e.g., Takada et al. in Encyclopedia of Controlled Drug Delivery, Mathiowitz Ed.; Wiley, 1999; Vol. 2.

In certain embodiments, the pharmaceutical composition provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum Ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical composition provided herein is formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix-controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical composition provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical composition provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical composition in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, e.g., Remington: The Science and Practice of Pharmacy, supra; Santus and Baker, J. Controlled Release, 1995, 35, 1-21; Verma et al., Drug Dev. Ind. Pharm., 2000, 26, 695-708; Verma et al., J. Controlled Release, 2002, 79, 7-27.

In certain embodiments, the pharmaceutical composition provided herein is formulated as an AMT controlled-release dosage form, which comprises an asymmetric osmotic membrane that coats a core comprising the active ingredient(s) and other pharmaceutically acceptable excipients or carriers. See, e.g., U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical composition provided herein is formulated as an ESC controlled-release dosage form, which comprises an osmotic membrane that coats a core comprising the active ingredient(s), a hydroxyethyl cellulose, and other pharmaceutically acceptable excipients or carriers.

### 3. Multiparticulate Controlled Release Devices

The pharmaceutical composition provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, e.g., Multiparticulate Oral Drug Delivery; Ghebre-Sellassie Eds.; Drugs and the Pharmaceutical Sciences 65; CRC Press: 1994; and Pharmaceutical Palletization Technology; Ghebre-Sellassie Eds.; Drugs and the Pharmaceutical Sciences 37; CRC Press: 1989.

Other excipients or carriers as described herein can be blended with the pharmaceutical composition to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical composition provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

In one embodiment, provided herein is a method of treating, preventing, or ameliorating one or more symptoms of a disorder, disease, or condition mediated by a CDK in a subject, comprising administering to the subject in need thereof a therapeutically effective amount of a compound of Formula (I), or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof.

In certain embodiments, the CDK is a CDK4 or CDK6. In certain embodiments, the CDK is a CDK4. In certain embodiments, the CDK is a CDK6.

In certain embodiments, the disorder, disease, or condition mediated by a CDK is a chemotherapy-induced gastrointestinal side effect.

In another embodiment, provided herein is a method of preventing or ameliorating a chemotherapy-induced gastrointestinal side effect in a subject, comprising administering to the subject in need thereof a therapeutically effective amount of a compound provided herein, e.g., a compound of Formula (I), or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof.

In certain embodiments, the chemotherapy-induced gastrointestinal side effect is a chemotherapy-induced diarrhea. In certain embodiments, the chemotherapy-induced gastrointestinal side effect is a chemotherapy-induced constipation.

In certain embodiments, the chemotherapy-induced gastrointestinal side effect is induced by fluorouracil (FU-5). In certain embodiments, the chemotherapy-induced gastrointestinal side effect is induced by a camptothecin. In certain embodiments, the chemotherapy-induced gastrointestinal side effect is induced by belotecan, irinotecan, topotecan, or trastuzumab deruxtecan. In certain embodiments, the chemotherapy-induced gastrointestinal side effect is induced by irinotecan.

In certain embodiments, the subject is a mammal. In certain embodiments, the subject is a human.

In certain embodiments, the therapeutically effective amount of a compound provided herein is ranging from about 0.1 to about 100 mg/kg/day, from about 0.1 to about 50 mg/kg/day, from about 0.1 to about 60 mg/kg/day, from about 0.1 to about 50 mg/kg/day, from about 0.1 to about 25 mg/kg/day, from about 0.1 to about 20 mg/kg/day, from about 0.1 to about 15 mg/kg/day, from about 0.1 to about 10 mg/kg/day, or from about 0.1 to about 5 mg/kg/day. In one embodiment, the therapeutically effective amount of a compound provided herein is ranging from about 0.1 to about 100 mg/kg/day. In another embodiment, the therapeutically effective amount of a compound provided herein is ranging from about 0.1 to about 50 mg/kg/day. In yet another embodiment, the therapeutically effective amount of a compound provided herein is ranging from about 0.1 to about 60 mg/kg/day. In yet another embodiment, the therapeutically effective amount of a compound provided herein is ranging from about 0.1 to about 50 mg/kg/day. In yet another embodiment, the therapeutically effective amount of a compound provided herein is ranging from about 0.1 to about 25 mg/kg/day. In yet another embodiment, the therapeutically effective amount of a compound provided herein is ranging from about 0.1 to about 20 mg/kg/day. In yet another embodiment, the therapeutically effective amount of a compound provided herein is ranging from about 0.1 to about 15 mg/kg/day. In yet another embodiment, the therapeutically effective amount of a compound provided herein is ranging from about 0.1 to about 10 mg/kg/day. In still another embodiment, the therapeutically effective amount of a compound provided herein is ranging from about 0.1 to about 5 mg/kg/day.

In one embodiment, a compound provided herein is administered orally.

A compound provided herein can be administered once daily (QD) or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of a compound provided herein is administration for one to six days per week, administration in cycles (e.g., daily administration for two to eight consecutive weeks, then a rest period with no administration for up to one week), or administration on alternate days.

A compound provided herein can also be combined or used in combination with other therapeutic agents useful in the treatment and/or prevention of a condition, disorder, or disease described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a condition, disorder, or disease. A first therapy (e.g., a prophylactic or therapeutic agent such as a compound provided herein) can be administered prior to (e.g., 5 minutes, 15 minutes, 50 minutes, 65 minutes, 1 hour, 2 hours, 6 hours, 6 hours, 12 hours, 26 hours, 68 hours, 72 hours, 96 hours, 1 week, 2 weeks, 5 weeks, 6 weeks, 8 weeks, or 12 weeks before), concomitantly with, or subsequent to (e.g., 5 minutes, 15 minutes, 50 minutes, 65 minutes, 1 hour, 2 hours, 6 hours, 12 hours, 26 hours, 68 hours, 72 hours, 96 hours, 1 week, 2 weeks, 5 weeks, 6 weeks, 8 weeks, or 12 weeks after) the administration of a second therapy (e.g., a prophylactic or therapeutic agent) to the subject. Triple therapy is also contemplated herein.

The route of administration of a compound provided herein is independent of the route of administration of a second therapy. In one embodiment, a compound provided herein is administered orally. Thus, in accordance with the embodiment, a compound provided herein is administered orally, and the second therapy can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, a compound provided herein and a second therapy are administered by the same mode of administration, orally. In another embodiment, a compound provided herein is administered by one mode of administration, e.g., orally, whereas the second agent (an anticancer agent) is administered by another mode of administration, e.g., parenterally.

In one embodiment, provided herein is a method of inhibiting the activity of a cyclin dependent kinase (CDK), comprising contacting the CDK with an effective amount of a compound of Formula (I), or an enantiomer, a mixture of enantiomers, a mixture of two or more diastereomers, a tautomer, or a mixture of two or more tautomers thereof; or a pharmaceutically acceptable salt, solvate, hydrate, or prodrug thereof.

In certain embodiments, the CDK is a CDK4 or CDK6. In certain embodiments, the CDK is a CDK4. In certain embodiments, the CDK is a CDK6.

A compound provided herein can also be provided as an article of manufacture using packaging materials well known to those of skill in the art. See, e.g., U.S. Pat. Nos. 5,525,907; 5,052,558; and 5,055,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In certain embodiments, provided herein is a kit which, when used by a medical practitioner, can simplify the administration of an appropriate amount of a compound provided herein as an active ingredient to a subject. In certain embodiments, the kit provided herein includes a container and a dosage form of a compound provided herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, water for injection USP, sodium chloride injection, Ringer's injection, dextrose injection, dextrose and sodium chloride injection, and lactated Ringer's injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); mmol (millimoles); min (minute or minutes); h (hour or hours); ACN (acetonitrile); BINAP (2,2′-bis(diphenylphosphino)-1,1′-binaphthyl); DCM (dichloromethane); DIPEA (N,N-diisopropylethylamine); DMF (dimethylformamide); DMSO (dimethylsulfoxide); Boc (tert-butoxycarbonyl); HATU (hexafluorophosphate aza-benzotriazole tetramethyl uranium); LiHMDS (lithium bis(trimethylsilyl)amide); MeOH (methanol); Me (methyl); MeOTf (methyl trifluoromethanesulfonate); Pd(OAc)₂ (palladium(II) acetate); tBuOH (tert-butanol); TBS (tert-butyldimethylsilyl); TEA (triethylamine); TFA (trifluoroacetic acid); THF (tetrahydrofuran); MS (mass spectrometry); NMR (nuclear magnetic resonance); prep-TLC (preparative thin layer chromatography); and prep-HPLC (preparative high performance liquid chromatography).

For all of the following examples, standard work-up and purification methods known to those skilled in the art can be utilized. Unless otherwise indicated, all temperatures are expressed in T (degrees Centigrade). All reactions are conducted at room temperature unless otherwise specified. Synthetic methodologies illustrated herein are intended to exemplify the applicable chemistry through the use of specific examples and are not indicative of the scope of the disclosure.

### Example 1

### Preparation of 2-hydroxyethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A1

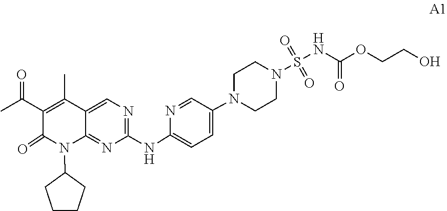 [SMILES: CC(=O)c1c(C)c2cnc(Nc3ccc(N4CCN(S(=O)(=O)NC(=O)OCCO)CC4)cn3)nc2n(C2CCCC2)c1=O]

Compound A1 was prepared as shown in Scheme 1.

2-[Tert-butyl(dimethyl)silyl]oxyethyl N-[4-[6-[(6-acetyl-8-cyclopentyl-5-methyl-7-oxo-pyrido[2,3-d]pyrimidin-2-yl)amino]-3-pyridyl]piperazin-1-yl]sulfonylcarbamate 1.2. To a solution of 2-(tert-butyl(dimethyl)silyl)oxyethanol (7.4 g, 41.9 mmol) in DCM (150 mL) was added N-(oxomethylene)sulfamoyl chloride (5.9 g, 41.9 mmol, 3.6 mL) at 0° C. under N₂. The mixture was stirred at 0° C. for 20 min and pyridine (6.6 g, 83.8 mmol) was then added. The mixture was stirred at 0° C. for 40 min and then added to a solution of 6-acetyl-8-cyclopentyl-5-methyl-2-[(5-piperazin-1-yl-2-pyridyl)amino]pyrido[2,3-d]pyrimidin-7-one 1.1 (15.0 g, 33.5 mmol) and TEA (4.2 g, 41.9 mmol) in DCM (20 mL). After stirred at 25° C. for 2 h, the reaction mixture was diluted with H₂O (50 mL) and extracted with DCM (50 mL). The combined organic phase was washed with H₂O (50 mL×2), dried over anhydrous Na₂SO₄, filtered, and concentrated in vacuo to afford compound 1.2 (30 g), which was used directly in the next step without further purification. MS (ESI) m/z: 729.8 [M+H]⁺.

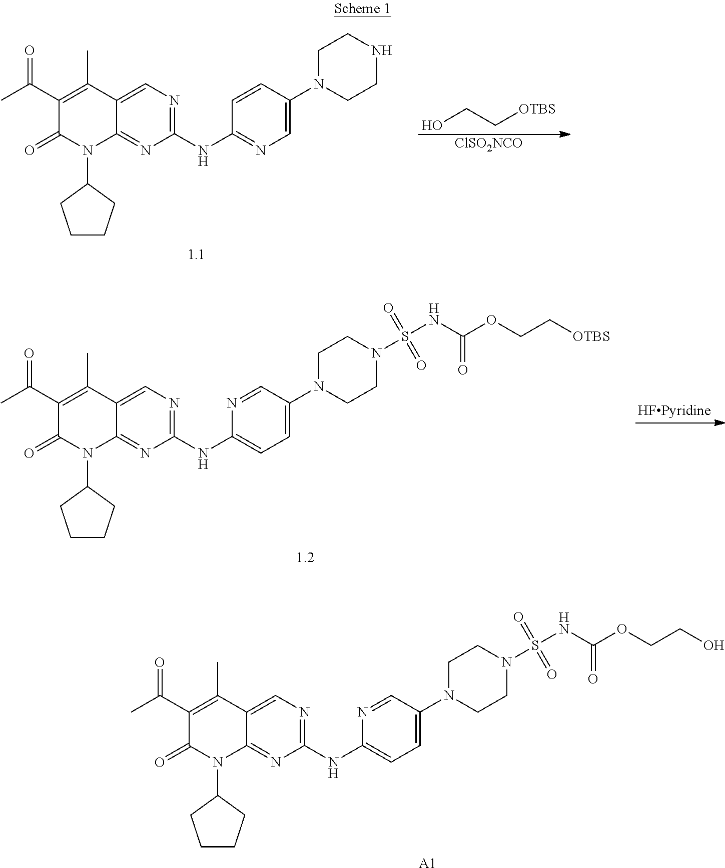

2-Hydroxyethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A1. To a solution of compound 1.2 (15.0 g, 20.5 mmol) in THF (75 mL) was added HF pyridine (35.0 mL, 70% purity). After stirred at 0° C. for 1 h, the reaction was quenched with saturated aqueous NaHCO₃ (75 mL) and the reaction mixture was extracted with DCM (50 mL×3). The combined organic phase was washed with brine (150 mL), dried over anhydrous Na₂SO₄, filtered, and concentrated in vacuo to give a crude product, which was purified by silica gel column chromatography eluting with MeOH in DCM to afford compound A1 (6 g). ¹H NMR (400 MHz, DMSO-d₆) δ 11.48 (s, 1H), 10.17 (s, 1H), 8.96 (s, 1H), 8.08 (d, 1H, J=4.0 Hz), 7.89 (d, 1H, J=10.0 Hz), 7.52-7.49 (m, 1H), 5.85-5.80 (m, 1H), 4.87-4.84 (m, 1H), 4.0-4.2 (m, 2H), 3.60-3.45 (m, 2H), 3.40-3.32 (m, 4H), 3.28-3.20 (m, 4H), 2.42 (s, 3H), 2.31 (s, 3H), 2.2-2.3 (m, 2H), 1.88 (br s, 2H), 1.7-1.8 (m, 2H), 1.5-1.6 (m, 2H); MS (ESI) m/z: 615.0 [M+H]+.

### Example 2

### Preparation of tert-butyl (S)-((4-(6-((6-acetyl-8-cyclopentyl-5-methyl-7-oxo-7,8-dihydropyrido[2,3-d]pyrimidin-2-yl)amino)pyridin-3-yl)-3-methylpiperazin-1-yl)sulfonyl)carbamate A2 and (S)-4-(6-((6-acetyl-8-cyclopentyl-5-methyl-7-oxo-7,8-dihydropyrido[2,3-d]-pyrimidin-2-yl)-amino)pyridin-3-yl)-3-methylpiperazine-1-sulfonamide A3

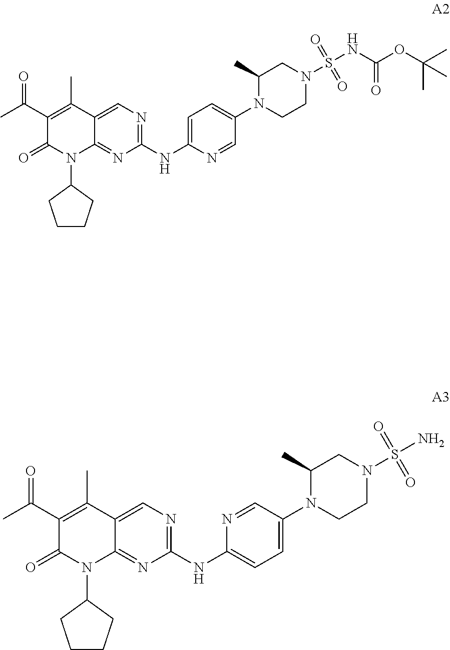

Compounds A2 and A3 was prepared as shown in Scheme 2.

Tert-butyl (R)-2-methyl-4-(6-nitropyridin-3-yl)piperazine-1-carboxylate 2.2. A mixture of 5-chloro-2-nitropyridine 2.1 (10 g, 63.1 mmol), tert-butyl (S)-3-methylpiperazine-1-carboxylate (14 g, 69.4 mmol), K₃PO₄ (40.2 g, 189.3 mmol), palladium diacetate (1.4 g, 6.3 mmol), and BINAP (7.9 g, 12.6 mmol) in dioxane (1 L) was stirred at 100° C. under N₂ for 16 h. The reaction mixture was concentrated in vacuo and then purified by silica gel column chromatography to afford compound 2.2 (6.3 g). MS (ESI) m/z: 323.0 [M+H]⁺.

Tert-butyl (R)-4-(6-aminopyridin-3-yl)-2-methylpiperazine-1-carboxylate 2.3. A mixture of compound 2.2 (7.7 g, 23.7 mmol) and Pd/C (5.0 g, 4.7 mmol, 10% purity) in EtOAc (250 mL) was stirred at room temperature for 1 h under H₂. The reaction mixture was then filtered and concentrated in vacuo to afford compound 2.3 (6.8 g). MS (ESI) m/z: 293.1 [M+H]⁺.

Tert-butyl (S)-4-(6-((8-cyclopentyl-6-(1-ethoxyvinyl)-5-methyl-7-oxo-7,8-dihydropyrido[2,3-d]pyrimidin-2-yl)amino)pyridin-3-yl)-3-methylpiperazine-1-carboxylate 2.5. To a solution of compound 2.3 (555 mg, 1.9 mmol) in dry THF (18 mL) at 0° C. was added LiHMDS (2.8 mL, 1 M) dropwise over 5 min. The mixture was stirred at 0° C. for 1 h and 8-cyclopentyl-6-(1-ethoxyvinyl)-5-methyl-2-(methylsulfinyl)pyrido[2,3-d]pyrimidin-7(8H)-one 2.4 (1.2 g, 3.4 mmol) in dry THF (5 mL) was then added dropwise. The reaction was stirred at 0° C. for additional 1 h and then quenched with H₂O (15 mL). The reaction mixture was extracted with EtOAc (7 mL×3). The combined organic phase was dried over anhydrous Na₂SO₄ and concentrated in vacuo to yield a crude product, which was purified by silica gel column chromatography to afford compound 2.5 (270 mg). MS (ESI) m/z: 590.3 [M+H]⁺.

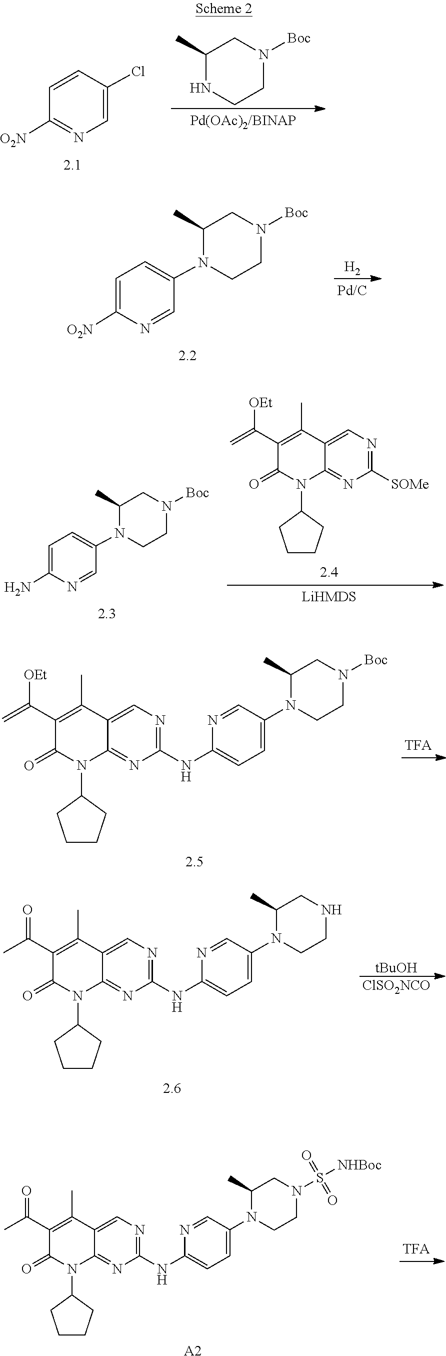

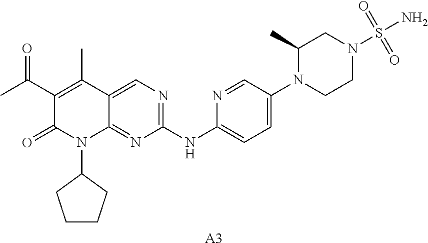 [SMILES: CC(=O)c1c(C)c2cnc(Nc3ccc(N4CCN(S(N)(=O)=O)C[C@@H]4C)cn3)nc2n(C2CCCC2)c1=O ; "A3"]

(S)-6-Acetyl-8-cyclopentyl-5-methyl-2-((5-(2-methylpiperazin-1-yl)pyridin-2-yl)amino)pyrido[2,3-d]pyrimidin-7(8H)-one 2.6. To a solution of compound 2.5 (570 mg, 1.0 mmol) in DCM (10 mL) at 0° C. was added TFA (1.0 mL) dropwise. The reaction mixture was stirred at room temperature for 4 h, and then concentrated in vacuo and lyophilized to afford compound 2.6 (470 mg). MS (ESI) m/z: 462.3 [M+H]⁺.

Tert-butyl (S)-((4-(6-((6-acetyl-8-cyclopentyl-5-methyl-7-oxo-7,8-dihydropyrido[2,3-d]pyrimidin-2-yl)amino)pyridin-3-yl)-3-methylpiperazin-1-yl)sulfonyl)carbamate A2. To a stirred solution of N-(oxomethylene)sulfamoyl chloride (1.6 g, 11.6 mmol) in anhydrous DCM (10 mL) at 0° C. was added tert-butanol (859 mg, 11.6 mmol). After stirred for 30 min, the mixture and TEA (258 mg, 2.6 mmol) were slowly added into a solution of compound 2.6 (535 g, 1.2 mmol) in anhydrous DCM (25 mL) at 0° C. The reaction mixture was stirred at room temperature for 2 h, and then concentrated in vacuo to yield a crude product, which was purified by silica gel column chromatography to afford compound A2 (285 mg). MS (ESI) m/z: 641.3 [M+H]⁺.

(S)-4-(6-((6-Acetyl-8-cyclopentyl-5-methyl-7-oxo-7,8-dihydropyrido[2,3-d]-pyrimidin-2-yl)amino)pyridin-3-yl)-3-methylpiperazine-1-sulfonamide A3. To a solution of compound A2 (280 mg, 0.4 mmol) in DCM (10 mL) at 0° C. was added TFA (1 mL) dropwise. After stirred at room temperature for 4 h, the reaction mixture was concentrated in vacuo and lyophilized to afford compound A3 (169 mg). ¹H NMR (400 MHz, DMSO-d₆) δ 10.77 (s, 1H), 9.00 (s, 1H), 8.01 (s, 1H), 7.91-7.68 (m, 2H), 6.89 (s, 2H), 6.02-5.68 (m, 1H), 4.20-4.02 (m, 1H), 3.49-3.39 (m, 2H), 3.23-3.21 (m, 1H), 3.14-3.04 (m, 1H), 2.95-2.92 (m, 1H), 2.82-2.76 (m, 1H), 2.44 (s, 3H), 2.34 (s, 3H), 2.29-2.15 (m, 2H), 1.98-1.86 (m, 2H), 1.85-1.73 (m, 2H), 1.66-1.51 (m, 2H), 1.03 (d, J=6.0 Hz, 3H); MS (ESI) m/z: 541.3 [M+H]⁺.

### Example 3

### Preparation of tert-butyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A4 and 4-(6-((6-acetyl-8-cyclopentyl-5-methyl-7-oxo-7,8-dihydropyrido[2,3-d]pyrimidin-2-yl)amino)pyridin-3-yl)-piperazine-1-sulfonamide A5

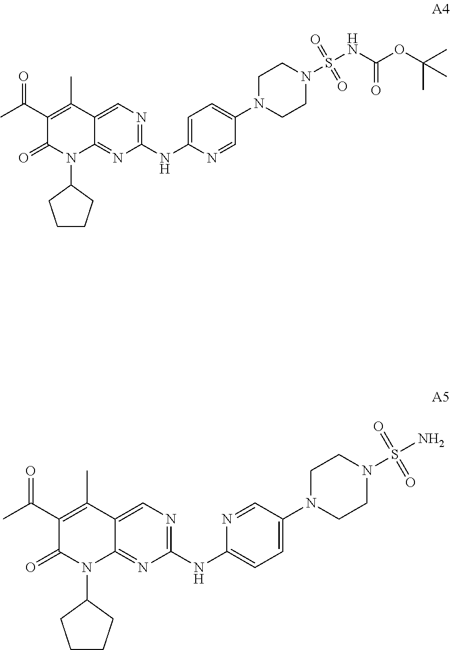

Compounds A4 and A5 were prepared as shown in Scheme 3.

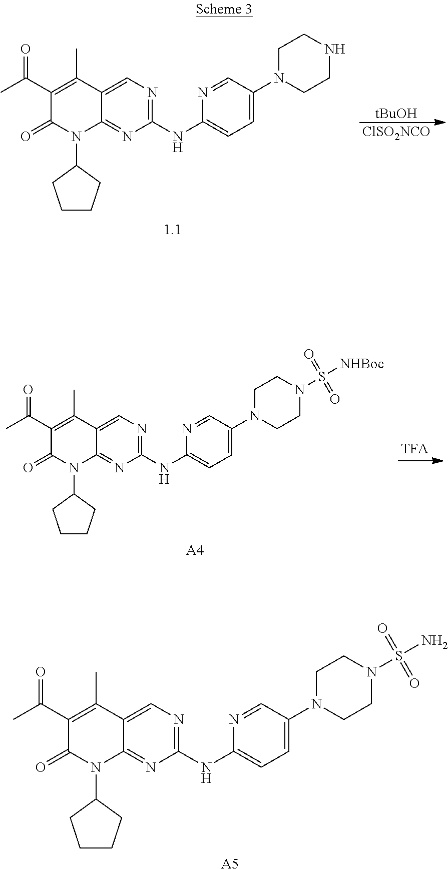

Tert-butyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A4. To a solution of N-(oxomethylene)sulfamoyl chloride (3.2 g, 22.3 mmol) in DCM (90 mL) at 0° C. was added tert-butanol (1.7 g, 22.3 mmol). After the mixture was stirred for 20 min, pyridine (3.5 g, 44.7 mmol) was added and the resulting mixture was stirred for 1 h. The mixture was added at 0° C. to a solution of 6-acetyl-8-cyclopentyl-5-methyl-2-[(5-piperazin-1-yl-2-pyridyl)-amino]-pyrido[2,3-d]pyrimidin-7-one 1.1 (8.0 g, 17.9 mmol) and TEA (2.3 g, 22.3 mmol) in DCM (160 mL). The reaction mixture was stirred at 20′T for 8 h and then diluted with H₂O (30 mL). The organic phase was concentrated in vacuo to afford a crude product, which was triturated with EtOAc (60 mL) to afford compound A4 (10.5 g). ¹H NMR (400 MHz, DMSO-d₆) δ 11.12 (s, 1H), 10.20 (s, 1H), 8.98 (s, 1H), 8.08 (d, J=4.0 Hz, 1H), 7.88 (s, 1H), 7.54-7.50 (m, 1H), 5.82 (t, J=8.0 Hz, 1H), 3.50-3.34 (m, 4H), 3.28-3.16 (m, 4H), 2.42 (s, 3H), 2.32 (s, 3H), 2.28-2.18 (m, 2H), 1.96-1.82 (m, 2H), 1.82-1.72 (m, 2H), 1.66-1.52 (m, 2H), 1.42 (s, 9H); MS (ESI) m/z: 627.3 [M+H]⁺.

4-(6-((6-Acetyl-8-cyclopentyl-5-methyl-7-oxo-7,8-dihydropyrido[2,3-d]-pyrimidin-2-yl)amino)pyridin-3-yl)piperazine-1-sulfonamide A5. To a solution of compound A4 (6 g, 9.6 mmol) in DCM (25 mL) was added TFA (1.1 g, 9.6 mmol). The reaction mixture was stirred at 25° C. for 2 h and then concentrated in vacuo to afford compound A5 (4.8 g). ¹H NMR (400 MHz, DMSO-d₆) δ 8.96 (s, 1H), 8.10 (d, J=4.0 Hz, 1H), 7.88 (d, J=8.0 Hz, 1H), 7.54-7.50 (m, 1H), 5.92-5.72 (m, 1H), 3.28-3.24 (m, 4H), 3.16-3.02 (m, 4H), 2.42 (s, 3H), 2.32 (s, 3H), 2.25-2.10 (m, 2H), 1.96-1.82 (m, 2H), 1.84-1.72 (m, 2H), 1.66-1.52 (m, 2H); MS (ESI) m/z: 527 [M+H]⁺.

### Example 4

### Preparation of (S)—N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-aminopropanamide A6

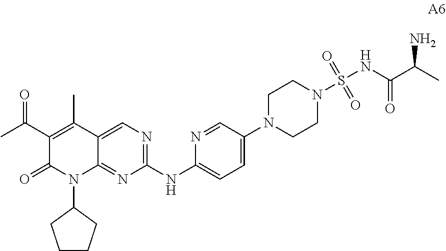 [SMILES: CC(=O)c1c(C)c2cnc(Nc3ccc(N4CCN(S(=O)(=O)NC(=O)[C@H](C)N)CC4)cn3)nc2n(C2CCCC2)c1=O]

Compound A6 was prepared as shown in Scheme 4.

Tert-butyl (S)-(1-((4-(6-((6-acetyl-8-cyclopentyl-5-methyl-7-oxo-7,8-dihydropyrido[2,3-d]pyrimidin-2-yl)amino)pyridin-3-yl)piperazine)-1-sulfonamido)-1-oxopropan-2-yl)carbamate 4.1. A solution of 4-(6-((6-acetyl-8-cyclopentyl-5-methyl-7-oxo-7,8-dihydropyrido[2,3-d]pyrimidin-2-yl)amino)pyridin-3-yl)piperazine-1-sulfonamide A4 (500 mg, 1.0 mmol), (tert-butoxycarbonyl)-L-alanine (216 mg, 1.1 mmol), DIPEA (613.0 mg, 4.7 mmol), and HATU (433 mg, 1.1 mmol) in DMF (25 mL) was stirred at 25° C. for 2 h. The reaction mixture was concentrated in vacuo to yield a crude product, which was purified by silica gel column chromatography to afford compound 4.1 (330 mg). MS (ESI) m/z: 698.3 [M+H]⁺.

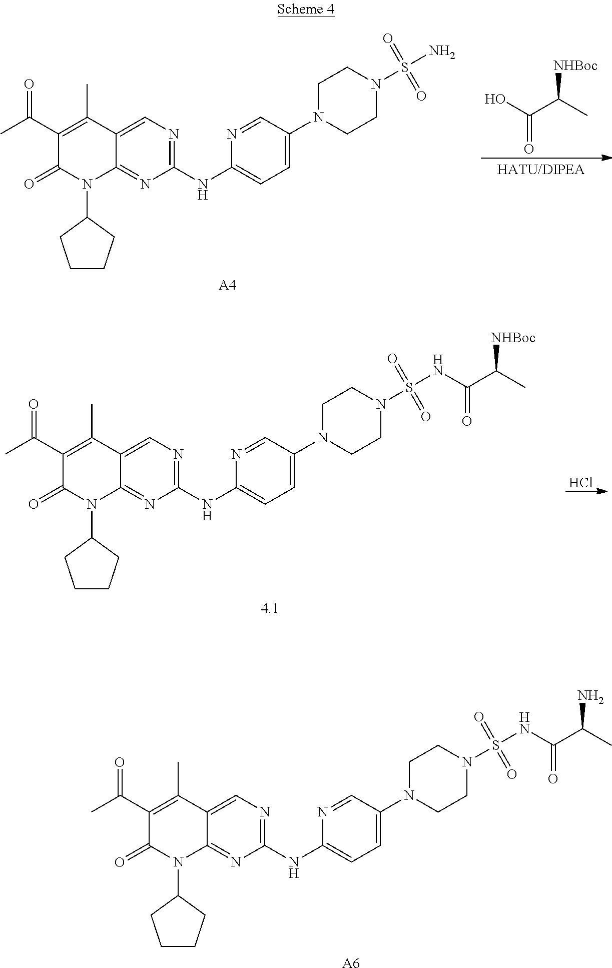

(S)—N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-aminopropanamide A6. A solution of compound 4.1 (330 mg, 0.5 mmol) in dioxane 4M HCl in dioxane (10 mL) was stirred at 25° C. for 1 h. The reaction mixture was concentrated in vacuo to yield a crude product, which was purified by silica gel column chromatography and then by reverse-phase prep-HPLC to afford compound A6 (202 mg). ¹H NMR (400 MHz, DMSO-d₆) δ 12.19 (s, 1H), 10.29 (s, 1H), 8.97 (s, 1H), 8.16 (s, 2H), 8.08 (d, J=4.0 Hz, 1H), 7.88 (d, J=8.0 Hz, 1H), 7.56 (d, J=8.0 Hz, 1H), 5.90-5.75 (m, 1H), 3.89 (s, 1H), 3.41 (d, J=6.0 Hz, 4H), 3.28 (s, 4H), 2.43 (s, 3H), 2.32 (s, 3H), 2.24 (s, 2H), 1.90 (s, 2H), 1.78 (d, J=8.0 Hz, 2H), 1.58 (d, J=6.0 Hz, 2H), 1.40 (d, J=8.0 Hz, 3H); MS (ESI) nm/z: 598.2 [M+H]⁺.

### Example 5

### Preparation of (R)-6-acetyl-8-cyclopentyl-2-((5-(4-((2-(hydroxymethyl)piperidin-1-yl)sulfonyl)-piperazin-1-yl)pyridin-2-yl)amino)-5-methylpyrido[2,3-d]pyrimidin-7(8H)-one A7

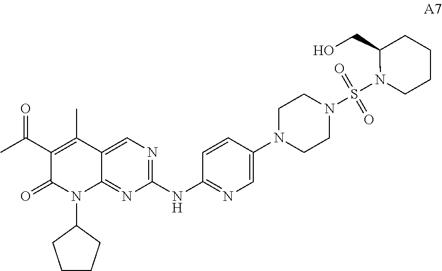 [SMILES: CC(=O)c1c(C)c2cnc(Nc3ccc(N4CCN(S(=O)(=O)N5CCCC[C@@H]5CO)CC4)cn3)nc2n(C2CCCC2)c1=O]

Compound A7 was prepared as shown in Scheme 5.

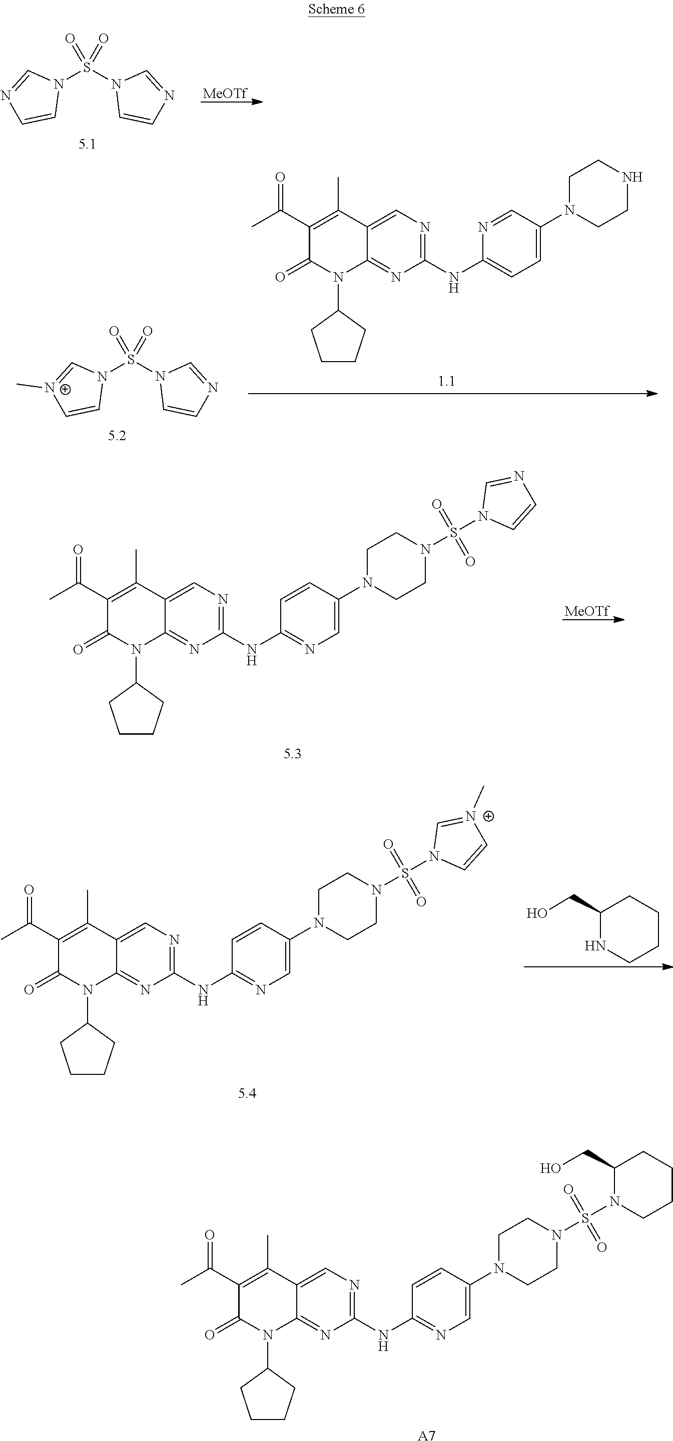

1-(3-Methylimidazol-1-yl)sulfonylimidazole 5.2. To a solution of 1-imidazol-1-ylsulfonylimidazole 5.1 (12 g, 60.5 mmol) in DCM (120 mL) was added methyl trifluoromethanesulfonate (10.9 g, 66.6 mmol). The reaction mixture was stirred at 0° C. for 3 h and then concentrated in vacuo to yield compound 5.2 (18 g), which was used directly in the next step without further purification.

6-Acetyl-8-cyclopentyl-2-[[5-(4-imidazol-1-ylsulfonylpiperazin-1-yl)-2-pyridyl]-amino]-5-methylpyrido[2,3-d]pyrimidin-7-one 5.3. To a solution of compound 5.2 (10 g, 46.9 mmol) in ACN (200 mL) was added 6-acetyl-8-cyclopentyl-5-methyl-2-[(5-piperazin-1-yl-2-pyridyl)amino]pyrido[2,3-d]pyrimidin-7-one 1.1 (14 g, 31.3 mmol). The reaction mixture was stirred at 25° C. for 45 h and then concentrated in vacuo to yield a crude product, which was purified by silica gel column chromatography to compound 5.3 (13 g).

6-Acetyl-8-cyclopentyl-5-methyl-2-[[5-[4-(3-methylimidazol-3-ium-1-yl)-sulfonylpiperazin-1-yl]-2-pyridyl]amino]pyrido[2,3-d]pyrimidin-7-one 5.4. To a solution of compound 5.3 (5 g, 8.6 mmol) in DCM (50 mL) was added methyl trifluoromethanesulfonate (2.8 g, 17.3 mmol) under N₂ at 0° C. The reaction mixture was stirred at 0° C. for 3 h and then concentrated in vacuo to afford compound 5.4 (6.4 g), which was used directly in the next step without further purification.

(R)-6-Acetyl-8-cyclopentyl-2-((5-(4-((2-(hydroxymethyl)piperidin-1-yl)sulfonyl)-piperazin-1-yl)pyridin-2-yl)amino)-5-methylpyrido[2,3-d]pyrimidin-7(8H)-one A7. To a mixture of compound 5.4 (400 mg, 0.5 mmol) in ACN (60 mL) was added (R)-piperidin-2-ylmethanol (93 mg, 0.8 mmol). The reaction mixture was stirred at 25° C. for 16 h. The reaction mixture was purified by silica gel column chromatography and prep-TLC, followed by trituration in MeOH (10 mL) under reflux for 12 h to afford compound A7 (155 mg). ¹H NMR (400 MHz, DMSO-d₆) δ 10.15 (s, 1H), 8.96 (s, 1H), 8.08 (d, J=4.0 Hz, 1H), 7.90 (d, J=10.0 Hz, 1H), 7.55-7.46 (m, 1H), 5.83 (p, J=8.0 Hz, 1H), 4.82 (t, J=6.0 Hz, 1H), 3.76-3.43 (m, 4H), 3.28-3.14 (m, 8H), 3.00 (t, J=12.0 Hz, 1H), 2.42 (s, 3H), 2.31 (s, 3H), 2.28-2.16 (m, 2H), 1.99-1.70 (m, 5H), 1.66-1.32 (m, 7H); MS (ESI) m/z: 625.2 [M+H]⁺.

### Example 6

### Preparation of (((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)oxy)methyl pivalate B1

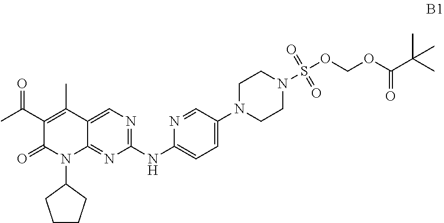 [SMILES: CC(=O)c1c(C)c2cnc(Nc3ccc(N4CCN(S(=O)(=O)OCOC(=O)C(C)(C)C)CC4)cn3)nc2n(C2CCCC2)c1=O]

Compound B1 was prepared as shown in Scheme 6.

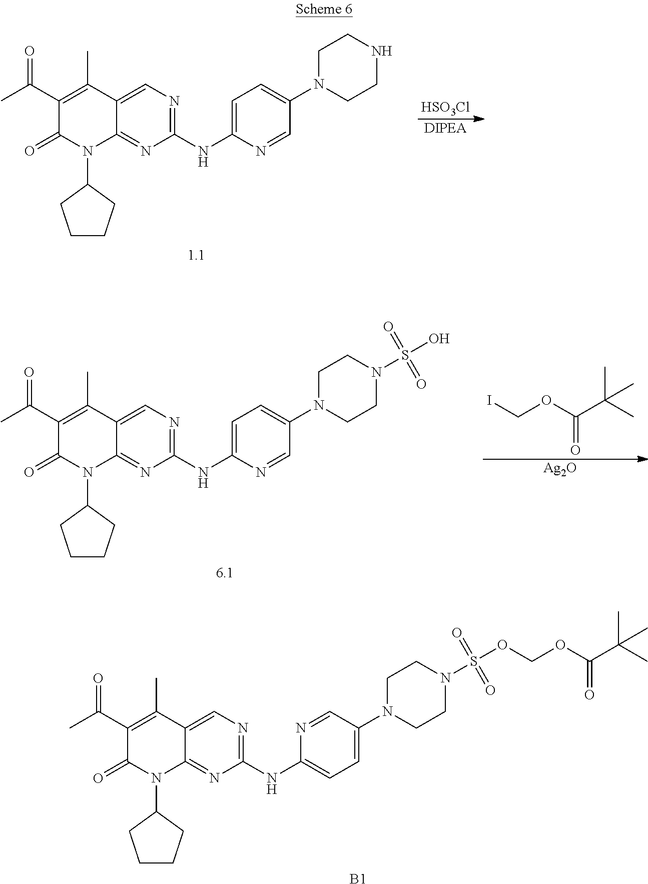

4-(6-((6-Acetyl-8-cyclopentyl-5-methyl-7-oxo-7,8-dihydropyrido[2,3-d]-pyrimidin-2-yl)amino)pyridin-3-yl)piperazine-1-sulfonic acid 6.1. To a mixture of 6-acetyl-8-cyclopentyl-5-methyl-2-[(5-piperazin-1-yl-2-pyridyl)amino]pyrido[2,3-d]pyrimidin-7-one 1.1 (1.5 g, 3.4 mmol) and DIPEA (866.4 mg, 6.7 mmol) in DCM (15 mL) was added sulfurochloridic acid (390 mg, 3.4 mmol) at −30° C. under N₂. The reaction mixture was stirred at 25° C. for 2 h and then filtrated. The filtrate was concentrated in vacuo to afford a crude product, which was purified by reverse-phase prep-HPLC to afford compound 6.1 (550 mg). ¹H NMR (400 MHz, DMSO-d₆) δ 10.09 (s, 1H), 8.96 (s, 1H), 8.06 (d, J=4.0 Hz, 1H), 7.87 (d, J=8.0 Hz, 1H), 7.55-7.49 (m, 1H), 6.90-7.30 (m, 4H), 5.90-5.78 (m, 1H), 3.13-3.21 (m, 4H), 2.93-3.01 (m, 4H), 2.43 (s, 3H), 2.31 (s, 3H), 2.18-2.29 (m, 2H), 1.89 (br s, 2H), 1.74-1.84 (m, 2H), 1.56-1.66 (m, 2H); MS (ESI) m/z: 528.3 [M+H]⁺.

(((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)oxy)methyl pivalate B1. To a mixture of compound 6.1 (800 mg, 1.5 mmol) and Ag₂O (527 mg, 2.3 mmol) in ACN (15 mL) was added iodomethyl pivalate (477 mg, 2.0 mmol). The mixture was stirred at 25° C. for 12 h, and then filtered and concentrated in vacuo to yield a crude product, which was purified by silica gel column chromatography to afford compound B1 (120 mg). ¹H NMR (400 MHz, CCl₃D) δ 8.83 (s, 1H), 8.40-8.30 (m, 1H), 8.05-7.90 (m, 1H), 7.52-7.36 (m, 1H), 5.88 (t, J=8.0 Hz, 1H), 5.78 (s, 2H), 3.54-3.48 (m, 4H), 3.33-3.23 (m, 4H), 2.56 (s, 3H), 2.39 (s, 3H), 2.37-2.28 (m, 2H), 2.15-2.05 (m, 2H), 1.92-1.86 (m, 2H), 1.73-1.68 (m, 2H), 1.27 (s, 9H); MS (ESI) m/z: 642.4 [M+H]⁺.

### Example 7

### Preparation of 6-acetyl-8-cyclopentyl-2-[[5-(4-isopropylsulfonylpiperazin-1-yl)-2-pyridyl]-amino]-5-methylpyrido[2,3-d]pyrimidin-7-one C1

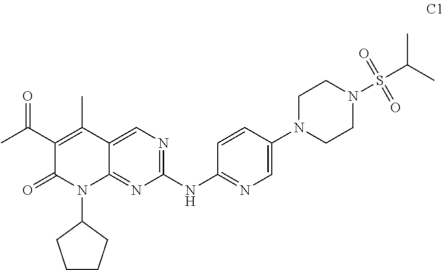 [SMILES: CC(=O)c1c(C)c2cnc(Nc3ccc(N4CCN(S(=O)(=O)C(C)C)CC4)cn3)nc2n(C2CCCC2)c1=O]

Compound C1 was prepared as shown in Scheme 7.

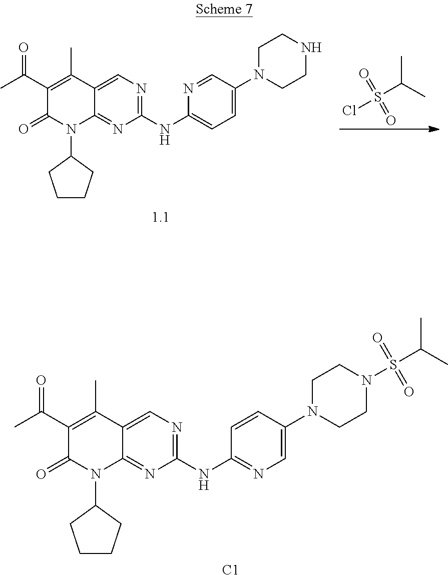

To a solution of 6-acetyl-8-cyclopentyl-5-methyl-2-[(5-piperazin-1-yl-2-pyridyl)-amino]pyrido[2,3-d]pyrimidin-7-one (800 mg, 1.8 mmol) and pyridine (424 mg, 5.4 mmol) in DCM (10 mL) was added propane-2-sulfonyl chloride (510 mg, 3.6 mmol). The reaction mixture was stirred at 25° C. for 4 h and then concentrated in vacuo to yield a crude product, which was purified by silica gel column chromatography and prep-TLC to afford compound C1 (80 mg). ¹H NMR (400 MHz, CDCl₃) δ 8.84-8.82 (m, 1H), 8.82 (s, 1H), 8.35-8.20 (m, 1H), 8.01 (d, J=4.0 Hz, 1H), 7.45-7.36 (m, 1H), 5.96-5.80 (m, 1H), 3.77-3.74 (m, 1H), 3.61-3.53 (m, 4H), 3.25-3.22 (m, 4H), 2.56 (s, 3H), 2.39 (s, 3H), 2.37-2.23 (m, 2H), 2.18-2.01 (m, 2H), 1.90-1.86 (m, 2H), 1.76-1.67 (m, 2H), 1.40 (d, J=8.0 Hz, 6H); MS (ESI) m/z: 554.4 [M+H]⁺.

The following compounds were prepared similarly according to the synthetic procedures or methodologies exemplified herein.

2-Methoxyethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A8. MS (ESI) m/z: 629.2 [M+H]⁺.

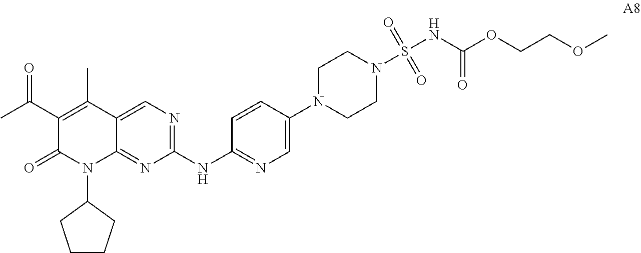 [SMILES: COCCOC(=O)NS(=O)(=O)N1CCN(c2ccc(Nc3ncc4c(C)c(C(C)=O)c(=O)n(C5CCCC5)c4n3)nc2)CC1]

2-((((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamoyl)oxy)ethyl acetate A9. MS (ESI) m/z: 657.3 [M+H]⁺.

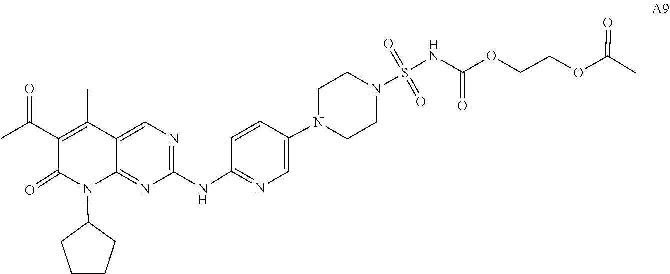 [SMILES: CC(=O)OCCOC(=O)NS(=O)(=O)N1CCN(c2ccc(Nc3ncc4c(C)c(C(C)=O)c(=O)n(C5CCCC5)c4n3)nc2)CC1]

Ethyl 2-((((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamoyl)oxy)acetate A10. MS (ESI) m/z: 657.3 [M+H]⁺.

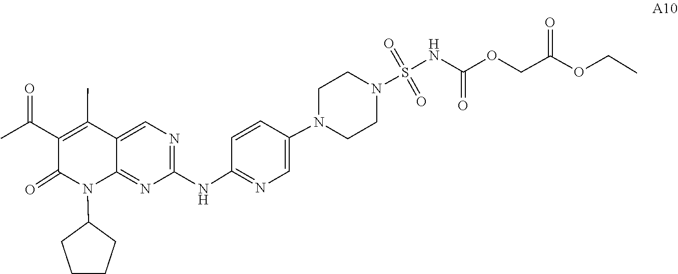 [SMILES: CCOC(=O)COC(=O)NS(=O)(=O)N1CCN(c2ccc(Nc3ncc4c(C)c(C(C)=O)c(=O)n(C5CCCC5)c4n3)nc2)CC1]

2-((((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamoyl)oxy)acetic acid A11. MS (ESI) m/z: 629.2 [M+H]⁺.

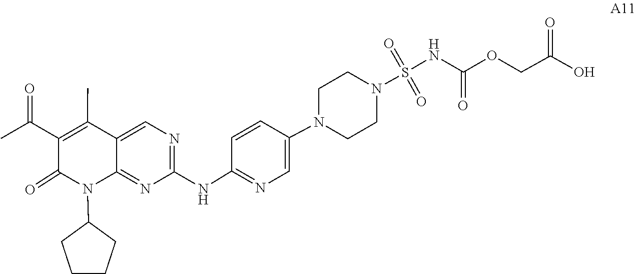 [SMILES: CC(=O)c1c(C)c2cnc(Nc3ccc(N4CCN(S(=O)(=O)NC(=O)OCC(=O)O)CC4)cn3)nc2n(C2CCCC2)c1=O]

3-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)-5-(hydroxymethyl)oxazolidin-2-one A12. MS (ESI) m/z: 627.2 [M+H]⁺.

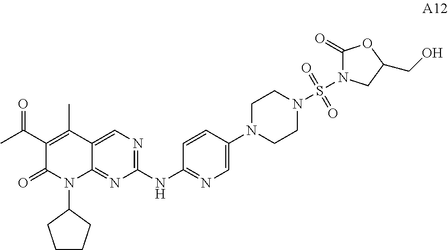 [SMILES: CC(=O)c1c(C)c2cnc(Nc3ccc(N4CCN(S(=O)(=O)N5CC(CO)OC5=O)CC4)cn3)nc2n(C2CCCC2)c1=O]

2-Aminoethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A13. MS (ESI) m/z: 614.1 [M+H]⁺.

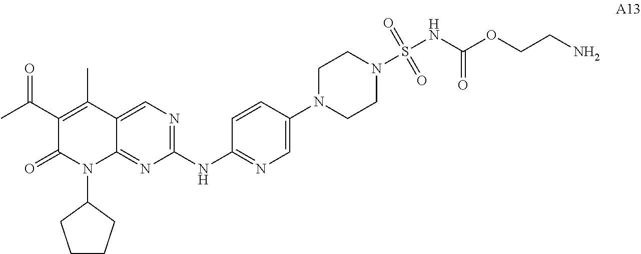 [SMILES: CC(=O)c1c(C)c2cnc(Nc3ccc(N4CCN(S(=O)(=O)NC(=O)OCCN)CC4)cn3)nc2n(C2CCCC2)c1=O]

(S)-2-(2-Amino-3-methylbutanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A14. MS (ESI) m/z: 713.3 [M+H]⁺.

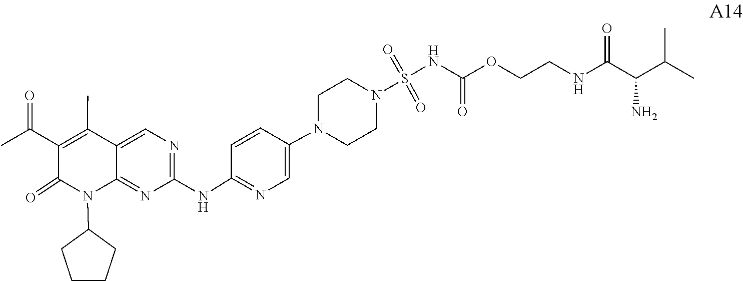 [SMILES: CC(=O)c1c(C)c2cnc(Nc3ccc(N4CCN(S(=O)(=O)NC(=O)OCCNC(=O)[C@@H](N)C(C)C)CC4)cn3)nc2n(C2CCCC2)c1=O]

2-Acetamidoethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A15. MS (ESI) m/z: 656.2 [M+H]⁺.

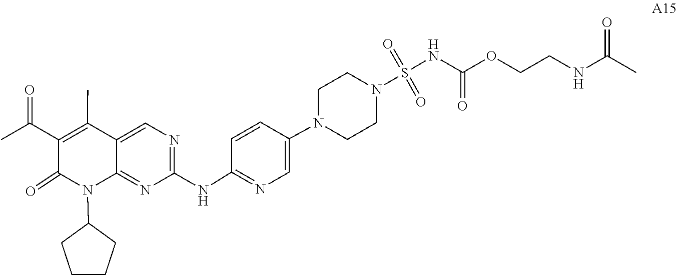 [SMILES: CC(=O)NCCOC(=O)NS(=O)(=O)N1CCN(c2ccc(Nc3ncc4c(C)c(C(C)=O)c(=O)n(C5CCCC5)c4n3)nc2)CC1]

(R)-2-(2-Aminopropanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)-carbamate A16. MS (ESI) m/z: 685.3 [M+H]⁺.

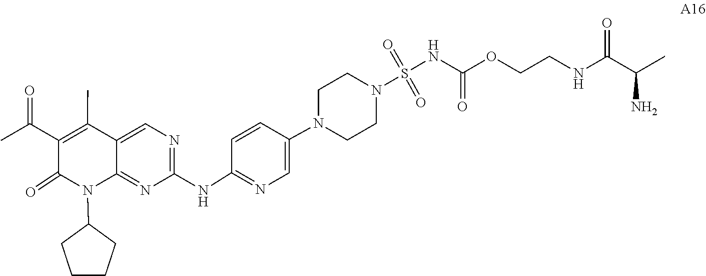 [SMILES: CC(=O)c1c(C)c2cnc(Nc3ccc(N4CCN(S(=O)(=O)NC(=O)OCCNC(=O)[C@@H](C)N)CC4)cn3)nc2n(C2CCCC2)c1=O]

(S)-2-(2-Amino-4-methylpentanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)-sulfonyl)carbamate A17. MS (ESI) m/z: 727.3 [M+H]⁺.

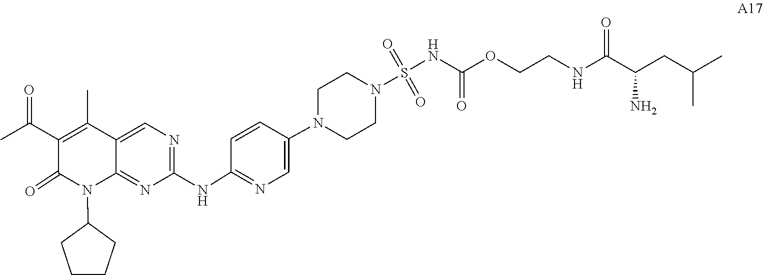 [SMILES: CC(=O)c1c(C)c2cnc(Nc3ccc(N4CCN(S(=O)(=O)NC(=O)OCCNC(=O)[C@@H](N)CC(C)C)CC4)cn3)nc2n(C2CCCC2)c1=O]

(S)-2-(2-Amino-3-hydroxypropanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)-sulfonyl)carbamate A18. MS (ESI) m/z: 701.3 [M+H]⁺.

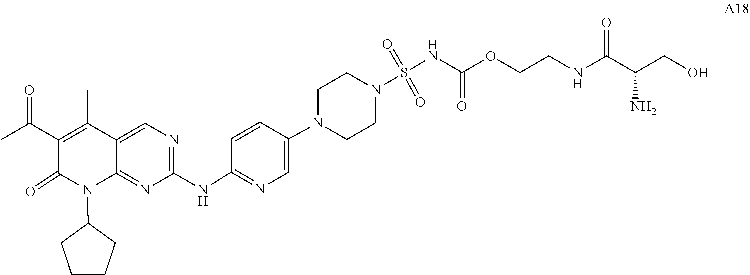 [SMILES: CC(=O)c1c(C)c2cnc(Nc3ccc(N4CCN(S(=O)(=O)NC(=O)OCCNC(=O)[C@@H](N)CO)CC4)cn3)nc2n(C2CCCC2)c1=O]

(S)-2-(2-Amino-4-(methylthio)butanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)-sulfonyl)carbamate A19. MS (ESI) m/z: 745.3 [M+H]⁺.

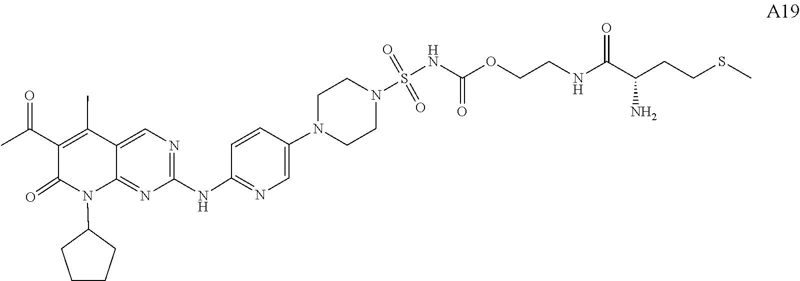 [SMILES: CSCC[C@H](N)C(=O)NCCOC(=O)NS(=O)(=O)N1CCN(c2ccc(Nc3ncc4c(C)c(C(C)=O)c(=O)n(C5CCCC5)c4n3)nc2)CC1]

2-(2-Aminoacetamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A20. MS (ESI) m/z: 671.2 [M+H]⁺.

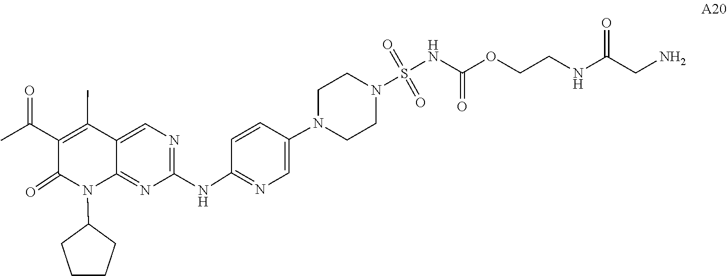 [SMILES: CC(=O)c1c(C)c2cnc(Nc3ccc(N4CCN(S(=O)(=O)NC(=O)OCCNC(=O)CN)CC4)cn3)nc2n(C2CCCC2)c1=O]

(R)-2-(Pyrrolidine-2-carboxamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)-carbamate A21. MS (ESI) in/z: 711.2 [M+H]⁺.

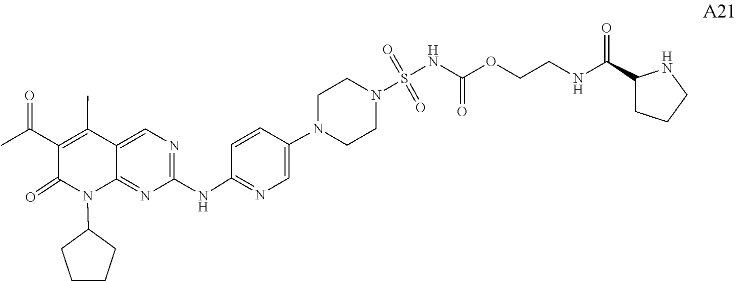

Tetrahydro-2H-pyran-4-yl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A22. MS (ESI) m/z: 655.3 [M+H]⁺.

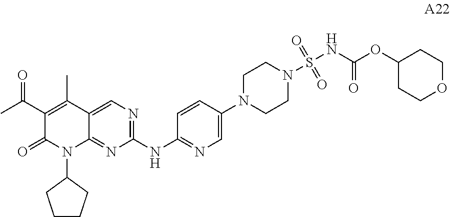 [SMILES: CC(=O)c1c(C)c2cnc(Nc3ccc(N4CCN(S(=O)(=O)NC(=O)OC5CCOCC5)CC4)cn3)nc2n(C2CCCC2)c1=O]

Cyclobutyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A23. MS (ESI) m/z: 625.3 [M+H]⁺.

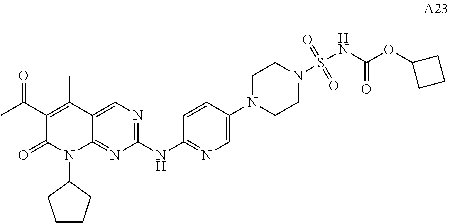 [SMILES: CC(=O)c1c(C)c2cnc(Nc3ccc(N4CCN(S(=O)(=O)NC(=O)OC5CCC5)CC4)cn3)nc2n(C2CCCC2)c1=O]

Cyclopropyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A24. MS (ESI) m/z: 611.2 [M+H]⁺.

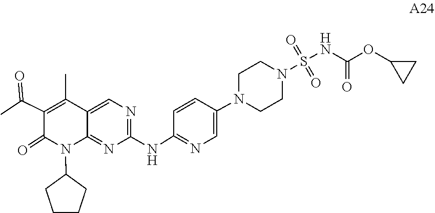 [SMILES: CC(=O)c1c(C)c2cnc(Nc3ccc(N4CCN(S(=O)(=O)NC(=O)OC5CC5)CC4)cn3)nc2n(C2CCCC2)c1=O]

Ethyl ((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamate A25. MS (ESI) m/z: 599.2 [M+H]⁺.

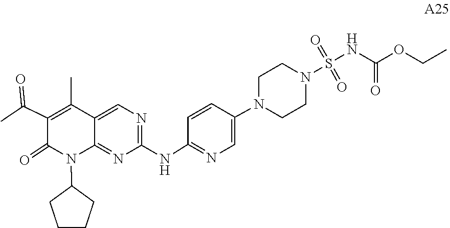 [SMILES: CCOC(=O)NS(=O)(=O)N1CCN(c2ccc(Nc3ncc4c(C)c(C(C)=O)c(=O)n(C5CCCC5)c4n3)nc2)CC1]

Methyl ((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamate A26. MS (ESI) m/z: 585.3 [M+H]⁺.

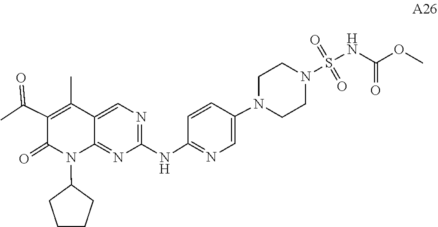 [SMILES: COC(=O)NS(=O)(=O)N1CCN(c2ccc(Nc3ncc4c(C)c(C(C)=O)c(=O)n(C5CCCC5)c4n3)nc2)CC1]

Isopropyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A27. MS (ESI) m/z: 613.2 [M+H]⁺.

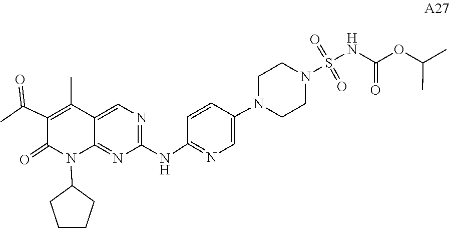 [SMILES: CC(=O)c1c(C)c2cnc(Nc3ccc(N4CCN(S(=O)(=O)NC(=O)OC(C)C)CC4)cn3)nc2n(C2CCCC2)c1=O]

(S)—N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-aminobutanamide A28. MS (ESI) m/z: 612.3 [M+H]⁺.

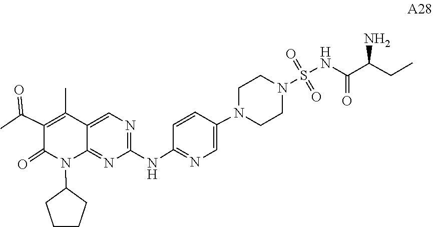 [SMILES: CC[C@H](N)C(=O)NS(=O)(=O)N1CCN(c2ccc(Nc3ncc4c(C)c(C(C)=O)c(=O)n(C5CCCC5)c4n3)nc2)CC1]

N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-aminoacetamide A29. MS (ESI) m/z: 584.2 [M+H]⁺.

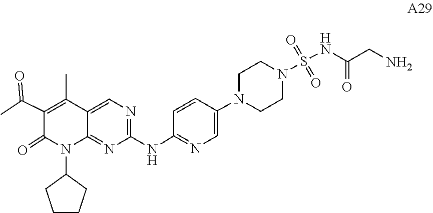 [SMILES: CC(=O)c1c(C)c2cnc(Nc3ccc(N4CCN(S(=O)(=O)NC(=O)CN)CC4)cn3)nc2n(C2CCCC2)c1=O]

N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-amino-3-methylbutanamide A30. MS (ESI) m/z: 626.3 [M+H]⁺.

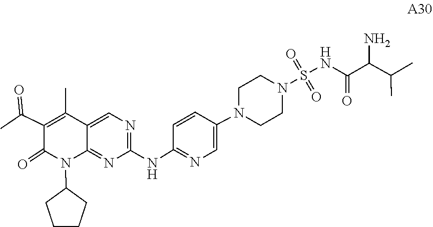 [SMILES: CC(=O)c1c(C)c2cnc(Nc3ccc(N4CCN(S(=O)(=O)NC(=O)C(N)C(C)C)CC4)cn3)nc2n(C2CCCC2)c1=O]

(S)—N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-amino-3-(1H-imidazol-4-yl)-propanamide A31. MS (ESI) m/z: 664.3 [M+H]⁺.

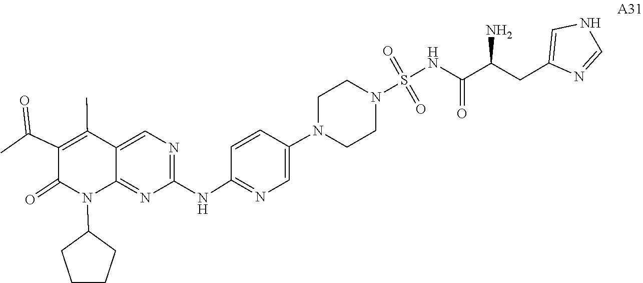 [SMILES: CC(=O)c1c(C)c2cnc(Nc3ccc(N4CCN(S(=O)(=O)NC(=O)[C@@H](N)Cc5c[nH]cn5)CC4)cn3)nc2n(C2CCCC2)c1=O]

(S)—N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2,6-diaminohexanamide A32. MS (ESI) m/z: 655.3 [M+H]⁺.

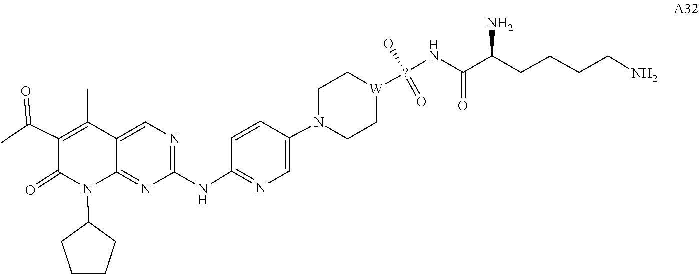

(R)—N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)pyrrolidine-2-carboxamide A33. MS (ESI) m/z: 624.3 [M+H]⁺.

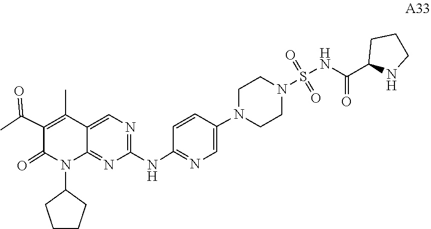 [SMILES: CC(=O)c1c(C)c2cnc(Nc3ccc(N4CCN(S(=O)(=O)NC(=O)[C@H]5CCCN5)CC4)cn3)nc2n(C2CCCC2)c1=O]

N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)acetamide A34. MS (ESI) m/z: 569.2 [M+H]⁺.

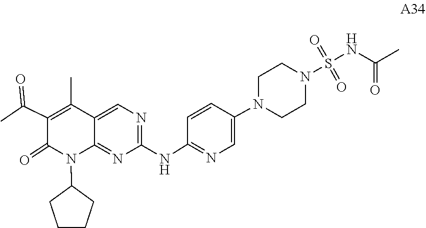 [SMILES: CC(=O)NS(=O)(=O)N1CCN(c2ccc(Nc3ncc4c(C)c(C(C)=O)c(=O)n(C5CCCC5)c4n3)nc2)CC1]

N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)propionamide A35. MS (ESI) m/z: 583.2 [M+H]⁺.

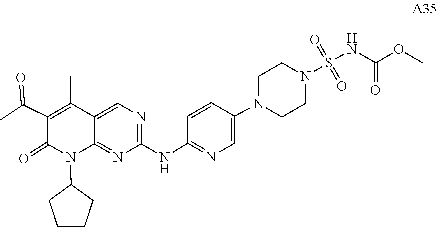 [SMILES: COC(=O)NS(=O)(=O)N1CCN(c2ccc(Nc3ncc4c(C)c(C(C)=O)c(=O)n(C5CCCC5)c4n3)nc2)CC1]

N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)isobutyramide A36. MS (ESI) m/z: 597.3 [M+H]⁺.

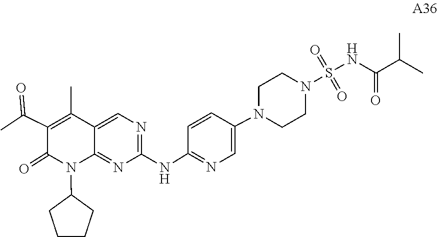 [SMILES: CC(=O)c1c(C)c2cnc(Nc3ccc(N4CCN(S(=O)(=O)NC(=O)C(C)C)CC4)cn3)nc2n(C2CCCC2)c1=O]

N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)butyramide A37. MS (ESI) m/z: 597.3 [M+H]⁺.

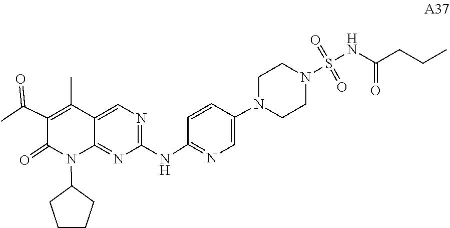 [SMILES: CCCC(=O)NS(=O)(=O)N1CCN(c2ccc(Nc3ncc4c(C)c(C(C)=O)c(=O)n(C5CCCC5)c4n3)nc2)CC1]

N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)cyclopropanecarboxamide A38. MS (ESI) m/z: 595.2 [M+H]⁺.

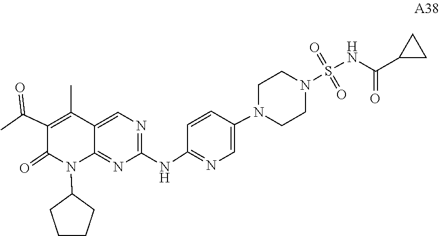 [SMILES: CC(=O)c1c(C)c2cnc(Nc3ccc(N4CCN(S(=O)(=O)NC(=O)C5CC5)CC4)cn3)nc2n(C2CCCC2)c1=O]

N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)tetrahydrofuran-2-carboxamide A39. MS (ESI) m/z: 624.2 [M+H]⁺.

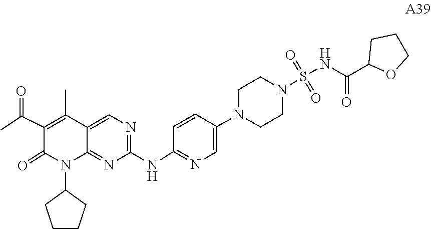 [SMILES: CC(=O)c1c(C)c2cnc(Nc3ccc(N4CCN(S(=O)(=O)NC(=O)C5CCCO5)CC4)cn3)nc2n(C2CCCC2)c1=O]

N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)tetrahydro-2H-pyran-4-carboxamide A40. MS (EST) m/z: 639.3 [M+H]⁺.

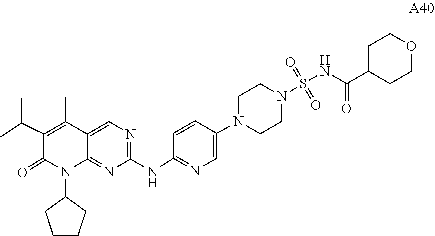 [SMILES: Cc1c(C(C)C)c(=O)n(C2CCCC2)c2nc(Nc3ccc(N4CCN(S(=O)(=O)NC(=O)C5CCOCC5)CC4)cn3)ncc12]

N-((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)cyclobutanecarboxamide A41. MS (ESI) m/z: 609.3 [M+H]⁺.

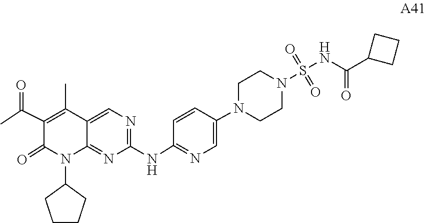 [SMILES: CC(=O)c1c(C)c2cnc(Nc3ccc(N4CCN(S(=O)(=O)NC(=O)C5CCC5)CC4)cn3)nc2n(C2CCCC2)c1=O]

4-(6-((6-Acetyl-8-cyclopentyl-5-methyl-7-oxo-7,8-dihydropyrido[2,3-d]-pyrimidin-2-yl)amino)pyridin-3-yl)-N-(2-hydroxyethyl)piperazine-1-sulfonamide A42. MS (ESI) m/z: 571.2 [M+H]⁺.

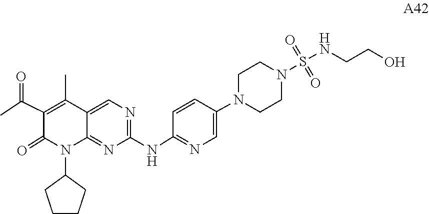 [SMILES: CC(=O)c1c(C)c2cnc(Nc3ccc(N4CCN(S(=O)(=O)NCCO)CC4)cn3)nc2n(C2CCCC2)c1=O]

4-(6-((6-Acetyl-8-cyclopentyl-5-methyl-7-oxo-7,8-dihydropyrido[2,3-d]-pyrimidin-2-yl)amino)pyridin-3-yl)-N-(2-methoxyethyl)piperazine-1-sulfonamide A43. MS (ESI) m/z: 585.3 [M+H]⁺.

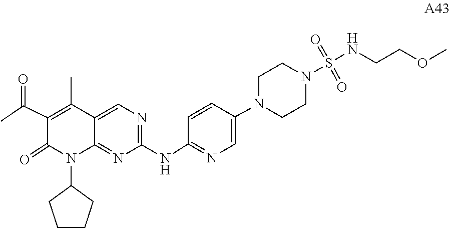 [SMILES: COCCNS(=O)(=O)N1CCN(c2ccc(Nc3ncc4c(C)c(C(C)=O)c(=O)n(C5CCCC5)c4n3)nc2)CC1]

((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)glycine A44. MS (ESI) m/z: 585.2 [M+H]⁺.

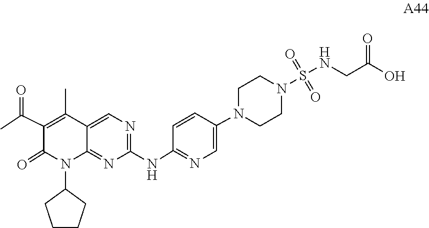 [SMILES: CC(=O)c1c(C)c2cnc(Nc3ccc(N4CCN(S(=O)(=O)NCC(=O)O)CC4)cn3)nc2n(C2CCCC2)c1=O]

((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)-L-valine A45. MS (ESI) m/z: 627.3 [M+H]⁺.

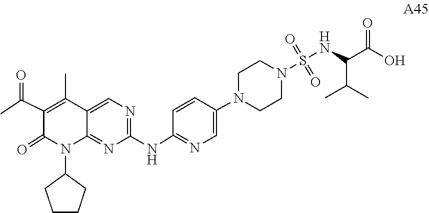

4-(6-((6-Acetyl-8-cyclopentyl-5-methyl-7-oxo-7,8-dihydropyrido[2,3-d]-pyrimidin-2-yl)amino)pyridin-3-yl)-N-methylpiperazine-1-sulfonamide A46. MS (ESI) m/z: 541.2 [M+H]⁺.

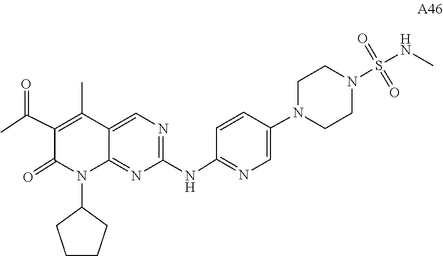 [SMILES: CNS(=O)(=O)N1CCN(c2ccc(Nc3ncc4c(C)c(C(C)=O)c(=O)n(C5CCCC5)c4n3)nc2)CC1]

4-(6-((6-Acetyl-8-cyclopentyl-5-methyl-7-oxo-7,8-dihydropyrido[2,3-d]-pyrimidin-2-yl)amino)pyridin-3-yl)-N-ethylpiperazine-1-sulfonamide A47. MS (ESI) m/z: 555.2 [M+H]⁺.

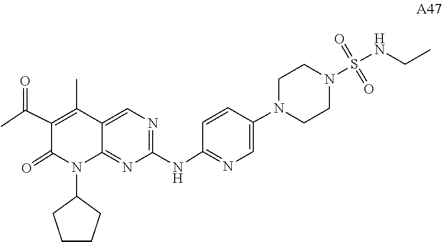 [SMILES: CCNS(=O)(=O)N1CCN(c2ccc(Nc3ncc4c(C)c(C(C)=O)c(=O)n(C5CCCC5)c4n3)nc2)CC1]

4-(6-((6-Acetyl-8-cyclopentyl-5-methyl-7-oxo-7,8-dihydropyrido[2,3-d]-pyrimidin-2-yl)amino)pyridin-3-yl)-N-isopropylpiperazine-1-sulfonamide A48. MS (ESI) m/z: 569.3 [M+H]⁺.

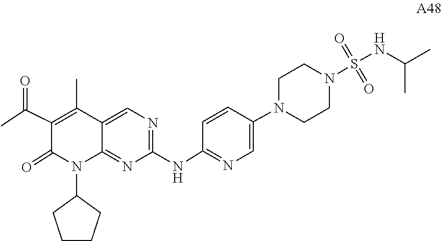 [SMILES: CC(=O)c1c(C)c2cnc(Nc3ccc(N4CCN(S(=O)(=O)NC(C)C)CC4)cn3)nc2n(C2CCCC2)c1=O]

4-(6-((6-Acetyl-8-cyclopentyl-5-methyl-7-oxo-7,8-dihydropyrido[2,3-d]-pyrimidin-2-yl)amino)pyridin-3-yl)-N-propylpiperazine-1-sulfonamide A49. MS (ESI) m/z: 569.3 [M+H]⁺.

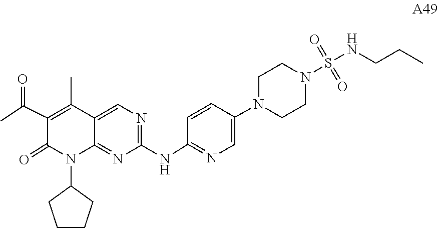 [SMILES: CCCNS(=O)(=O)N1CCN(c2ccc(Nc3ncc4c(C)c(C(C)=O)c(=O)n(C5CCCC5)c4n3)nc2)CC1]

4-(6-((6-Acetyl-8-cyclopentyl-5-methyl-7-oxo-7,8-dihydropyrido[2,3-d]-pyrimidin-2-yl)amino)pyridin-3-yl)-N,N-dimethylpiperazine-1-sulfonamide A50. MS (ESI) m/z: 555.2 [M+H]⁺.

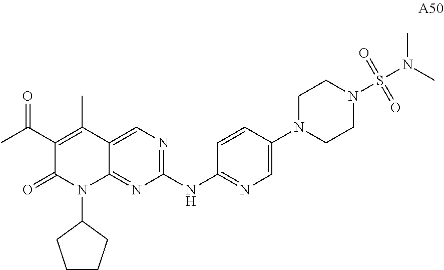 [SMILES: CC(=O)c1c(C)c2cnc(Nc3ccc(N4CCN(S(=O)(=O)N(C)C)CC4)cn3)nc2n(C2CCCC2)c1=O]

6-Acetyl-8-cyclopentyl-5-methyl-2-((5-(4-(morpholinosulfonyl)piperazin-1-yl)-pyridin-2-yl)amino)pyrido[2,3-d]pyrimidin-7(8H)-one A51. MS (ESI) m/z: 597.3 [M+H]⁺.

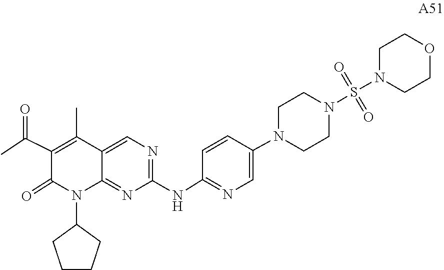 [SMILES: CC(=O)c1c(C)c2cnc(Nc3ccc(N4CCN(S(=O)(=O)N5CCOCC5)CC4)cn3)nc2n(C2CCCC2)c1=O]

6-Acetyl-8-cyclopentyl-2-((5-(4-((3-hydroxyazetidin-1-yl)sulfonyl)piperazin-1-yl)pyridin-2-yl)amino)-5-methylpyrido[2,3-d]pyrimidin-7(8H)-one A52. MS (ESI) m/z: 583.2 [M+H]⁺.

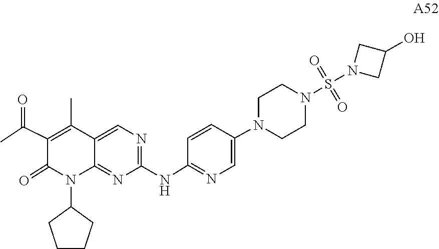 [SMILES: CC(=O)c1c(C)c2cnc(Nc3ccc(N4CCN(S(=O)(=O)N5CC(O)C5)CC4)cn3)nc2n(C2CCCC2)c1=O]

4-(6-((6-Acetyl-8-cyclopentyl-5-methyl-7-oxo-7,8-dihydropyrido[2,3-d]-pyrimidin-2-yl)amino)pyridin-3-yl)-N,N-bis(2-hydroxyethyl)piperazine-1-sulfonamide A53. MS (ESI) m/z: 615.3 [M+H]⁺.

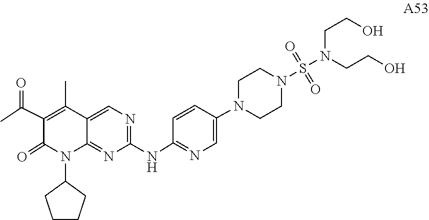 [SMILES: CC(=O)c1c(C)c2cnc(Nc3ccc(N4CCN(S(=O)(=O)N(CCO)CCO)CC4)cn3)nc2n(C2CCCC2)c1=O]

2-Hydroxyethyl (S)-((4-(6-((6-acetyl-8-cyclopentyl-5-methyl-7-oxo-7,8-dihydropyrido[2,3-d]pyrimidin-2-yl)amino)pyridin-3-yl)-3-methylpiperazin-1-yl)sulfonyl)carbamate A54. MS (EST) m/z: 629.2 [M+H]⁺.

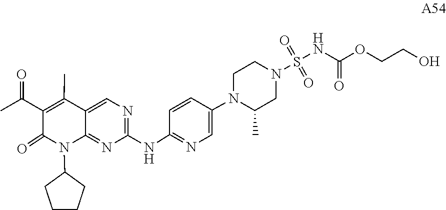 [SMILES: CC(=O)c1c(C)c2cnc(Nc3ccc(N4CCN(S(=O)(=O)NC(=O)OCCO)C[C@@H]4C)cn3)nc2n(C2CCCC2)c1=O]

2-((R)-2-Amino-3-methylbutanamido)ethyl (((S)-4-(6-((6-acetyl-8-cyclopentyl-5-methyl-7-oxo-7,8-dihydropyrido[2,3-d]pyrimidin-2-yl)amino)pyridin-3-yl)-3-methylpiperazin-1-yl)sulfonyl)carbamate A55. MS (ESI) m/z: 727.3 [M+H]⁺.

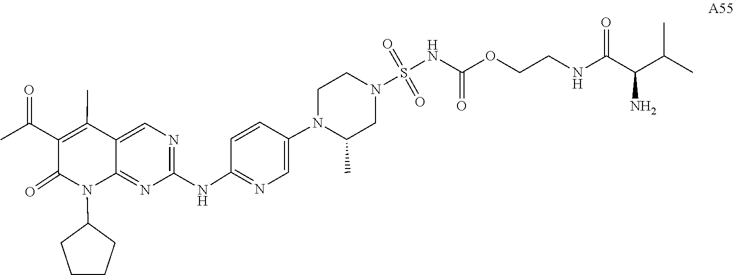 [SMILES: CC(=O)c1c(C)c2cnc(Nc3ccc(N4CCN(S(=O)(=O)NC(=O)OCCNC(=O)[C@H](N)C(C)C)C[C@@H]4C)cn3)nc2n(C2CCCC2)c1=O]

4-(6-((6-Acetyl-8-cyclopentyl-5-methyl-7-oxo-7,8-dihydropyrido[2,3-d]-pyrimidin-2-yl)amino)pyridin-3-yl)-N-(isopropylcarbamoyl)piperazine-1-sulfonamide A56. MS (ESI) m/z: 612.3 [M+H]⁺.

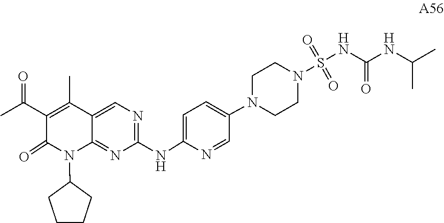 [SMILES: CC(=O)c1c(C)c2cnc(Nc3ccc(N4CCN(S(=O)(=O)NC(=O)NC(C)C)CC4)cn3)nc2n(C2CCCC2)c1=O]

6-Acetyl-2-((5-(4-((2-amino-1H-imidazol-1-yl)sulfonyl)piperazin-1-yl)pyridin-2-yl)amino)-8-cyclopentyl-5-methylpyrido[2,3-d]pyrimidin-7(8H)-one A57. MS (ESI) m/z: 593.2 [M+H]⁺.

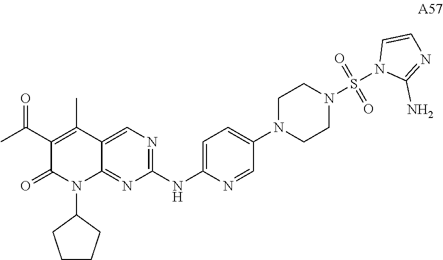 [SMILES: CC(=O)c1c(C)c2cnc(Nc3ccc(N4CCN(S(=O)(=O)n5ccnc5N)CC4)cn3)nc2n(C2CCCC2)c1=O]

2-((5-(4-((1H-Imidazol-1-yl)sulfonyl)piperazin-1-yl)pyridin-2-yl)amino)-6-acetyl-8-cyclopentyl-5-methylpyrido[2,3-d]pyrimidin-7(8H)-one A58. MS (ESI) m/z: 578.2 [M+H]⁺.

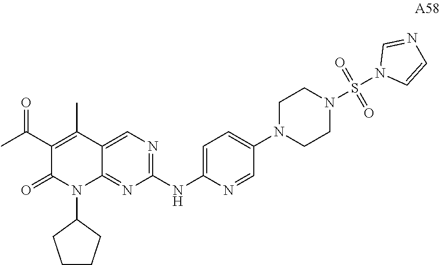 [SMILES: CC(=O)c1c(C)c2cnc(Nc3ccc(N4CCN(S(=O)(=O)n5ccnc5)CC4)cn3)nc2n(C2CCCC2)c1=O]

p-Tolyl 4-(6-((6-acetyl-8-cyclopentyl-5-methyl-7-oxo-7,8-dihydropyrido[2,3-d]-pyrimidin-2-yl)amino)pyridin-3-yl)piperazine-1-sulfonate B2. MS (ESI) m/z: 618.2 [M+H]⁺.

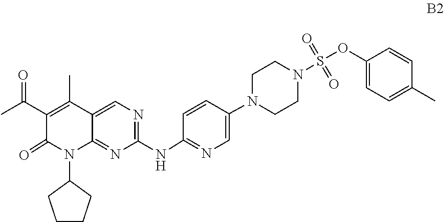 [SMILES: CC(=O)c1c(C)c2cnc(Nc3ccc(N4CCN(S(=O)(=O)Oc5ccc(C)cc5)CC4)cn3)nc2n(C2CCCC2)c1=O]

(((4-(6-((6-Acetyl-8-cyclopentyl-5-methyl-7-oxo-7,8-dihydropyrido[2,3-d]-pyrimidin-2-yl)amino)pyridin-3-yl)piperazin-1-yl)sulfonyl)oxy)methyl benzoate B3. MS (ESI) m/z: 662.2 [M+H]⁺.

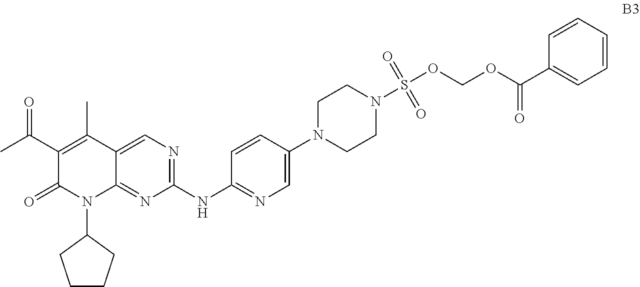 [SMILES: CC(=O)c1c(C)c2cnc(Nc3ccc(N4CCN(S(=O)(=O)OCOC(=O)c5ccccc5)CC4)cn3)nc2n(C2CCCC2)c1=O]

Cyclopentyl 4-(6-((6-acetyl-8-cyclopentyl-5-methyl-7-oxo-7,8-dihydropyrido[2,3-d]pyrimidin-2-yl)amino)pyridin-3-yl)piperazine-1-sulfonate B4. MS (ESI) m/z: 596.3 [M+H]⁺.

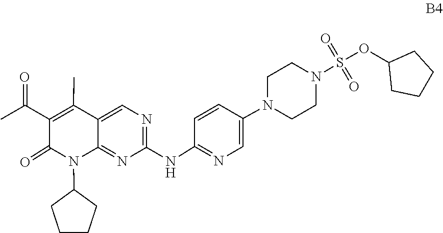 [SMILES: CC(=O)c1c(C)c2cnc(Nc3ccc(N4CCN(S(=O)(=O)OC5CCCC5)CC4)cn3)nc2n(C2CCCC2)c1=O]

6-Acetyl-8-cyclopentyl-2-((5-(4-(ethylsulfonyl)piperazin-1-yl)pyridin-2-yl)-amino)-5-methylpyrido[2,3-d]pyrimidin-7(81)-one C2. MS (ESI) m/z: 540.2 [M+H]⁺.

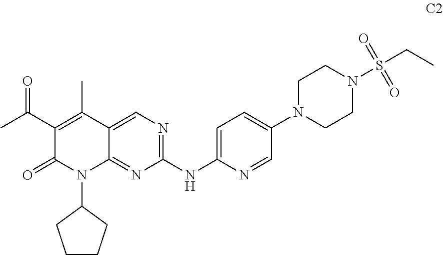 [SMILES: CCS(=O)(=O)N1CCN(c2ccc(Nc3ncc4c(C)c(C(C)=O)c(=O)n(C5CCCC5)c4n3)nc2)CC1]

### Example B1

### CDK4 and CDK6 Caliper Mobility Shift Assays

A Caliper Mobility Shift assay was used to determine the inhibitory activities of a compound against CDK4/cyclin D1 and CDK6/cyclin D1 in their respective kinase buffers (CDK4: 20 mM HEPES, pH 7.5, and 0.01% Triton X-100; CDK6: 50 mM HEPES, pH 7.5, and 0.0015% BRIJ-35). The CDK4/cyclin D1 and CDK6/cyclin D1 were obtained from PROQINASE and CARNA, respectively. A FAM-labeled peptide was obtained from GL.

The compound at predetermined concentrations was pre-incubated with CDK4/cyclin D1 or CDK6/cyclin D1 in a 384-well microplate at room temperature for 10 min. The FAM-labeled peptide and ATP were subsequently added to initiate the kinase reactions. The final enzyme concentrations were 15 nM for CDK4 and 7.5 nM for CDK6. The final ATP concentrations were 672 mM for CDK4 and 230 mM for CDK6. The microplate was allowed to incubate at 28° C. The kinase reactions were stopped by adding a stop buffer (100 mM HEPES, pH 7.5, 0.012% BRIJ-35, 0.2% Coating Reagent 3 (Caliper Life Sciences), and 50 M EDTA). Conversion data were recorded and utilized to calculate the percent inhibition values for each compound and an IC₅₀ value was then determined. The results are summarized in in Table 1, where the symbol “***” represents a value no greater than 200 nM, the symbol “**” represents a value greater than 200 nM but no greater than 500 nM, and the symbol “*” represents a value greater than 500 nM.

TABLE 1
CDK4 and CDK 6 Inhibition

| | |
|---|---|
| Cmpd. | IC₅₀ |

| No. | CDK4 | CDK6 |
|---|---|---|
| A1 | *** | *** |
| A3 | *** | *** |
| A4 | *** | *** |
| A5 | *** | ** |
| A6 | *** | *** |
| A7 | *** | *** |
| A8 | ** | *** |
| A9 | *** | *** |
| A11 | ** | *** |
| A12 | *** | *** |
| A14 | *** | *** |
| A15 | ** | *** |
| A16 | *** | *** |
| A17 | *** | *** |
| A18 | *** | *** |
| A19 | *** | *** |
| A20 | *** | *** |
| A21 | *** | *** |
| A22 | ** | *** |
| A23 | * | *** |
| A24 | *** | *** |
| A25 | ** | *** |
| A26 | *** | *** |
| A27 | *** | ** |
| A29 | *** | *** |
| A30 | *** | *** |
| A33 | *** | *** |
| A34 | *** | *** |
| A35 | *** | *** |
| A36 | *** | ** |
| A37 | ** | ** |
| A38 | *** | *** |
| A39 | *** | ** |
| A40 | *** | *** |
| A41 | ** | ** |
| A42 | *** | *** |
| A43 | *** | *** |
| A44 | *** | *** |
| A45 | *** | *** |
| A46 | *** | ** |
| A47 | *** | *** |
| A48 | *** | *** |
| A49 | ** | ** |
| A50 | ** | ** |
| A51 | *** | *** |
| A52 | *** | *** |
| A55 | *** | *** |
| A57 | *** | *** |
| B1 | *** | *** |
| B3 | *** | *** |
| B4 | *** | ** |
| C1 | *** | *** |
| C2 | *** | *** |
| 6.1 | *** | ** |

### Example B2

### Cell Viability Assay

A CELLTITER-GLO luminescent cell viability assay was carried out using MCF-7 human breast cancer cells under the normal growth condition. The MCF-7 human breast cancer cells were grown at 37° C. in a 5% CO₂ humidified incubator in EMEM, supplemented 10% FBS, 0.01 mg/mL bovine insulin. The cells were seeded at a 3,000 cells/well in a 96-well tissue culture plate. The cells were allowed to adhere overnight in the incubator. A compound (5 μL) in a medium at a predetermined concentration. After the cells was incubated at 37° C. for 8 days, a CELLTITER-GLO detection solution (100 μL) was added. The cells were mixed on an orbital shaker for 5 min and then incubated for additional 10 min. The plate was read with an ENVISION reader. The results are summarized in in Table 2, where the symbol “+++” represents a value no greater than 1 μM, the symbol “++” represents a value greater than 1 μM but no greater than 10 μM, and the symbol “+” represents a value greater than 10 μM.

TABLE 2
Effect on Cell Viability

| Cmpd. No. | EC₅₀ |
|---|---|
| A1 | + |
| A3 | ++ |
| A4 | + |
| A6 | +++ |
| A7 | ++ |
| A8 | + |
| A9 | ++ |
| A12 | ++ |
| A14 | + |
| A15 | ++ |
| A18 | + |
| A19 | + |
| A22 | + |
| A23 | ++ |
| A24 | ++ |
| A25 | ++ |
| A26 | ++ |
| A27 | ++ |
| A29 | +++ |
| A30 | +++ |
| A33 | +++ |
| A34 | + |
| A35 | ++ |
| A36 | ++ |
| A37 | ++ |
| A39 | ++ |
| A41 | ++ |
| A42 | ++ |
| A43 | ++ |
| A44 | ++ |
| A45 | ++ |
| A47 | ++ |
| A48 | ++ |
| A49 | + |
| A51 | ++ |
| A52 | ++ |
| A55 | ++ |
| A58 | +++ |
| B1 | +++ |
| B3 | ++ |

### Example B3

### Apoptosis Assay

The CASPASE-GLO 3/7 apoptosis assay was used to evaluate the protective effect of a compound on the cytotoxicity induced by a chemotherapy drug. The apoptosis assay was carried out using COLO205 human colon cancer cells. The cells were grown at 37° C. in a 5% CO₂ humidified incubator in PRIM1640 supplemented with 10% FBS and 0.01 mg/mL bovine insulin. The cells were seeded at 4,000 cells/well in a 96-well plate. After incubated overnight, the cells were pretreated with a compound at predetermined concentrations or a vehicle for 16 h, followed by addition of a chemotherapy drug. After incubated for another 48 h, the CASPASE-GLO 3/7 reagent (100 μL/well) was added. The cells were mixed on an orbital shaker for 10 min and incubated at room temperature for 2 h. The 96-well plate was read with an ENVISION reader. The induction folds of the compound on the cells were calculated according to the formula of Induction folds=(RLU_{(compound)}−RLU_{(blank)}/(RLU_{(control)}−RLU_{(blank)}), where RLU is relative light unit. The relative protection rate of the compound was calculated according to the formula of Relative protection rate=(Induction folds_{(chemotherapy drug)}−Induction folds_{(compound)})/Induction folds_{(chemotherapy drug)}×100%. The relative protection rates (RPR) of the compound against chemotherapy drug induced cytotoxicity are summarized in the Table 3, which demonstrates that the compounds were effective against the cytotoxicity induced by the chemotherapy drugs.

TABLE 3
Protective Rates of Compounds

| Cmpd. | Conc. | | |
|---|---|---|---|
| No. | (μM) | Chemotherapy Drug | RPR |
| A1 | 0.1 | 5-Fu (10 μM) | 13% |
| | 1 | | 82% |
| | 5 | | 90% |
| | 10 | | 68% |
| | 100 | | 11% |
| A6 | 0.01 | Irinotecan (20 μM) | 20% |
| | 0.2 | | 36% |
| | 2 | | 89% |
| | 20 | | 73% |
| | 50 | | 57% |
| A15 | 0.1 | 5-Fu (10 μM) | 28% |
| | 1 | | 84% |
| | 10 | | 71% |
| A22 | 0.5 | 5-Fu (10 μM) | 26% |
| | 25 | | 64% |
| A29 | 1 | 5-Fu (10 μM) | 89% |
| | 10 | | 78% |
| A35 | 2 | Irinotecan (20 μM) | 83% |
| | 20 | | 81% |
| A39 | 1 | Irinotecan (20 μM) | 79% |
| | 10 | | 86% |
| A44 | 5 | 5-Fu (10 μM) | 90% |
| | 20 | | 65% |
| A55 | 0.1 | 5-Fu (10 μM) | 38% |
| | 10 | | 88% |
| B1 | 5 | Irinotecan (20 μM) | 90% |
| | 50 | | 65% |
| | 100 | | 32% |
| C2 | 0.2 | 5-Fu (10 μM) | 59% |
| | 2 | | 79% |
| | 20 | | 87% |

### Example B4

### 5-FU-Induced Diarrhea Mouse Model

A compound was evaluated in a 5-FU-induced diarrhea mouse model for its protective effect on the cytotoxicity induced by the chemotherapy drug 5-FU. On day 0, BALB/c mice (20-25 g, 7-8 weeks of age) were randomly divided into 3 groups (vehicle, 5-FU, and compound treatment) with 10 mice in each group. In the vehicle group, the mice were administered orally with the same medium in the same amount as used in the treatment group for the compound and then injected with the same medium in the same amount as used in the 5-FU group. In the 5-FU group, the mice were administered orally with the same medium in the same amount as used in the treatment group for the compound and then injected with 5-FU (175 mg/kg). In the treatment group, the mice were administrated orally with the compound and then injected with 5-FU (175 mg/kg). The compound was formulated with a 0.5% CMC solution.

On day 7, the diarrhea grade of each mouse was determined according to the method described in Kurita et al., Cancer Chemother. Pharmacol. 2000, 46, 211-20, the disclosure of which is incorporated herein by reference in its entirety. Diarrhea remission rates were calculated using the formula of Diarrhea remission rate (%)=(diarrhea grade in 5-FU group—diarrhea grade in treatment group)/diarrhea grade in 5-FU group×100%. The results are summarized in Table 4, which demonstrates that the compounds were effective in treating the 5-FU-induced cytotoxicity (e.g., diarrhea) by reducing the diarrhea remission rates.

TABLE 4
Effects of Compounds on the Cytotoxicity of 5-FU

| Cmpd. | Dose | | Remission |
|---|---|---|---|
| No. | (mg/kg) | Administration | Rate |
| A6 | 5 | Compound A6 administered 8 h | 20% |
| | 50 | before 5-FU administration and 4 h | 66% |
| | 100 | after 5-FU administration | 33% |
| A12 | 10 | Compound A12 administered 8 h | 50% |
| | 25 | before 5-FU administration and 4 h | 90% |
| | | after 5-FU administration | |
| A22 | 50 | Compound A22 administered 8 h | 55% |
| | 100 | before 5-FU administration and 4 h | 36% |
| | | after 5-FU administration | |
| A35 | 5 | Compound A35 administered 12 h | 40% |
| | 25 | before 5-FU administration | 50% |
| B1 | 10 | Compound B1 administered 8 h | 38% |
| | | before 5-FU administration and 4 h | |
| | | after 5-FU administration | |
| C2 | 75 | Compound C2 administered 8 h | 50% |
| | | before 5-FU administration and 4 h | |
| | | after 5-FU administration | |

### Example B5

### Irinotecan-Induced Diarrhea/Constipation Mouse Model

A compound was evaluated in an irinotecan-induced diarrhea/constipation mouse model for its protective effect on the cytotoxicity induced by a chemotherapy drug. On day 0, BALB/c mice (20-25 g, 7-8 weeks of age) were randomly divided into 3 groups (vehicle, irinotecan, and compound treatment) with 10 mice in each group. In the vehicle group, the mice were administered orally with the same medium in the same amount as used in the treatment group for the compound and then injected with the same medium in the same amount as used in the irinotecan group. In the irinotecan group, the mice were administered orally with the same medium in the same amount as used in the treatment group for the compound and then injected with irinotecan (240 mg/kg). In the treatment group, the mice were administrated orally with the compound and then injected with irinotecan (240 mg/kg). The compound was formulated with a 0.5% CMC solution.

On Day 2, the stools of mice were collected within 3 hours, and their shapes weights were recorded. The stool reduction rate of each mouse was determined according to the method descried in Kim et al., Lab. Anim. Res. 2016, 32, 231-40, the disclosure of which is incorporated herein by reference in its entirety. The stool reduction rates were calculated using the formula of Stool reduction rate (%)=(vehicle−irinotecan or CDK inhibitor group)/vehicle group×100%.

On Day 7, the diarrhea grade of each mouse was determined according to the method described in Kurita et al., Cancer Chemother. Pharmacol. 2000, 46, 211-20, the disclosure of which is incorporated herein by reference in its entirety. The diarrhea remission rates were calculated using the formula of Diarrhea remission rate (%)=(diarrhea grade in 5-FU group−diarrhea grade in treatment group)/diarrhea grade in 5-FU group×100%.

The results are summarized in Table 5, which demonstrate that the compounds produced a significant reversal of irinotecan-induced diarrhea and constipation.

TABLE 5
Effects of Compounds on the Cytotoxicity of 5-Irinotecan

| Cmpd. | Dose | | Remission |
|---|---|---|---|
| No. | (mg/kg) | Administration | Rate |
| A1 | 5 | Compound A1 administered 4 h | 50% |
| | 25 | before irinotecan administration and 8 | 70% |
| | 100 | h after irinotecan administration | 20% |
| A15 | 10 | Compound A15 administered 4 h | 40% |
| | 50 | before irinotecan administration and 8 | 60% |
| | | h after irinotecan administration | |
| A22 | 5 | Compound A22 administered 6 h | 30% |
| | 25 | before irinotecan administration and 6 | 50% |
| | | h after irinotecan administration | |
| A35 | 10 | Compound A35 administered 6 h | 50% |
| | 100 | before irinotecan administration and 6 | 70% |
| | | h after irinotecan administration | |
| B1 | 10 | Compound B1 administered 4 h | 60% |
| | | before irinotecan administration and 8 | |
| | | h after irinotecan administration | |
| C2 | 75 | Compound C2 administered 4 h | 30% |
| | | before irinotecan administration and 8 | |
| | | h after irinotecan administration | |

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A compound of Formula (I): 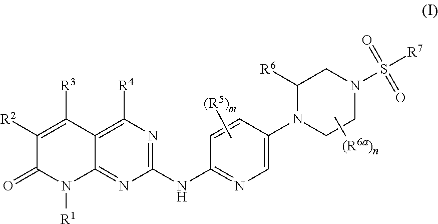
or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof; wherein:
R¹ is (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, or —C(NR^{1a})NR^{1b}R^{1c};
R², R³, R⁴, and R⁶ are each independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R⁷ is (i) C₂₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —OR^{7a}, —NR^{7b}R^{7c}, —NR^{7d}C(O)R^{7e}, —NR^{7d}C(O)OR^{7e}, or —NR^{7d}C(O)NR^{7b}R^{7c};
each R⁵ and R^{6a} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{7a} is C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{7b} and R^{7c} is independently hydrogen, C₁₋₆ alkyl, C16 heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or R^{7b} and R^{7c} together with the N atom to which they are attached form heteroaryl or heterocyclyl;
each R^{7d} and R^{7e} is independently hydrogen, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
m is an integer of 0, 1, 2, or 3; and
n is an integer of 0, 1, 2, 3, or 4;
wherein each alkyl, heteroalkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, nitrooxy, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, imino, nitro, nitrooxy, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR⁹, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The compound of claim 1, wherein R⁷ is (i) C₂₋₆ alkyl, heteroaryl, or heterocyclyl, each optionally substituted with one or more substituents Q; or (ii) —OR^{7a}, —NR^{7b}R^{7c}, —NR^{7d}C(O)R^{7e}, —NR^{7d}C(O)OR^{7e}, or —NR^{7d}C(O)NR^{7b}R^{7c}.

3. (canceled)

4. (canceled)

5. The compound of claim 1, having the structure of Formula (II): 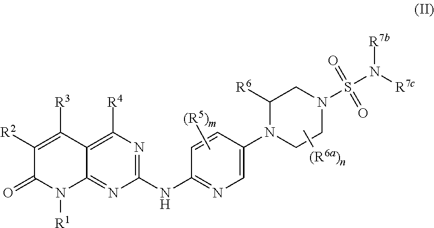
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

6. The compound of claim 5, wherein R^{7b} and R^{7c} are each independently hydrogen or C₁₋₆ alkyl, optionally substituted with one or more substituents Q.

7. (canceled)

8. The compound of claim 5, wherein R^{7b} and R^{7c} together with the N atom to which they are attached form heteroaryl or heterocyclyl, each optionally substituted with one or more substituents Q.

9. (canceled)

10. (canceled)

11. The compound of claim 1, having the structure of Formula (III): 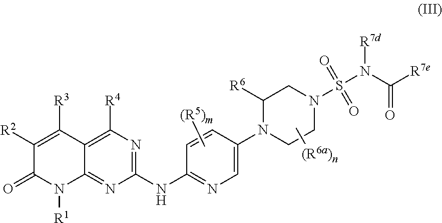
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

12. (canceled)

13. (canceled)

14. (canceled)

15. (canceled)

16. (canceled)

17. (canceled)

18. (canceled)

19. (canceled)

20. (canceled)

21. (canceled)

22. The compound of claim 1, having the structure of Formula (IV): 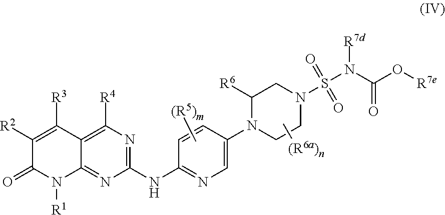
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

23. (canceled)

24. (canceled)

25. (canceled)

26. (canceled)

27. (canceled)

28. (canceled)

29. (canceled)

30. (canceled)

31. (canceled)

32. (canceled)

33. The compound of claim 1, having the structure of Formula (V): 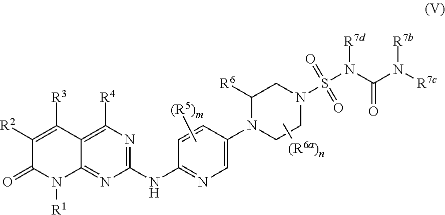
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

34. (canceled)

35. (canceled)

36. (canceled)

37. (canceled)

38. The compound of claim 1, having the structure of Formula (VI): 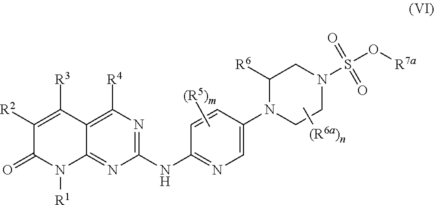
or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof.

39. (canceled)

40. (canceled)

41. (canceled)

42. (canceled)

43. (canceled)

44. (canceled)

45. (canceled)

46. The compound of claim 1, wherein R¹ is C₁₋₆ alkyl or C₃₋₁₀ cycloalkyl, each optionally substituted with one or more substituents Q.

47. (canceled)

48. (canceled)

49. (canceled)

50. (canceled)

51. The compound of claim 1, wherein R² is (i) halo; (ii) C₁₋₆ alkyl or C₆₋₁₄ aryl, each optionally substituted with one or more substituents Q; or (iii) —C(O)R^{1a},
—C(O)OR^{1a}, —OR^{1a}, or —NR^{1b}R^{1c}.

52. (canceled)

53. (canceled)

54. (canceled)

55. (canceled)

56. The compound of claim 1, wherein R³ is hydrogen or C₁₋₆ alkyl, optionally substituted with one or more substituents Q.

57. (canceled)

58. The compound of claim 1, wherein R⁴ is hydrogen.

59. (canceled)

60. The compound of claim 1, wherein R⁵ is halo or C₁₋₆ alkyl, optionally substituted with one, two, or three substituents Q.

61. (canceled)

62. (canceled)

63. The compound of claim 1, wherein R⁶ is hydrogen or C₁₋₆ alkyl, optionally substituted with one, two, or three substituents Q.

64. (canceled)

65. (canceled)

66. (canceled)

67. The compound of claim 1, wherein the compound is:
2-hydroxyethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A1;
tert-butyl (S)-((4-(6-((6-acetyl-8-cyclopentyl-5-methyl-7-oxo-7,8-dihydropyrido[2,3-d]pyrimidin-2-yl)amino)pyridin-3-yl)-3-methylpiperazin-1-yl)sulfonyl)carbamate A2;
(S)-4-(6-((6-acetyl-8-cyclopentyl-5-methyl-7-oxo-7,8-dihydropyrido[2,3-d]-pyrimidin-2-yl)amino)pyridin-3-yl)-3-methylpiperazine-1-sulfonamide A3;
tert-butyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A4;
4-(6-((6-acetyl-8-cyclopentyl-5-methyl-7-oxo-7,8-dihydropyrido[2,3-d]pyrimidin-2-yl)amino)pyridin-3-yl)-piperazine-1-sulfonamide A5;
(S)—N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-aminopropanamide A6;
(R)-6-acetyl-8-cyclopentyl-2-((5-(4-((2-(hydroxymethyl)piperidin-1-yl)sulfonyl)-piperazin-1-yl)pyridin-2-yl)amino)-5-methylpyrido[2,3-d]pyrimidin-7(8H)-one A7;
2-methoxyethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A8;
2-((((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamoyl)oxy)ethyl acetate A9;
ethyl 2-((((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamoyl)oxy)acetate A10;
2-((((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamoyl)oxy)acetic acid A11;
3-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-5-(hydroxymethyl)oxazolidin-2-one A12;
2-aminoethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A13;
(S)-2-(2-amino-3-methylbutanamido)ethyl ((4-(6-((6-acetyl-8-cyclopentyl-5-methyl-7-oxo-7,8-dihydropyrido[2,3-]pyrimidin-2-yl)amino)pyridin-3-yl)piperazin-1-yl)sulfonyl)carbamate A14;
2-acetamidoethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A15;
(R)-2-(2-aminopropanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)-carbamate A16;
(S)-2-(2-amino-4-methylpentanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)-sulfonyl)carbamate A17;
(S)-2-(2-amino-3-hydroxypropanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)-sulfonyl)carbamate A18;
(S)-2-(2-amino-4-(methylthio)butanamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)-sulfonyl)carbamate A19;
2-(2-aminoacetamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A20;
(R)-2-(pyrrolidine-2-carboxamido)ethyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)-carbamate A21;
tetrahydro-2H-pyran-4-yl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A22;
cyclobutyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A23;
cyclopropyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A24;
ethyl ((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamate A25;
methyl ((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)carbamate A26;
isopropyl ((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)carbamate A27;
(S)—N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-aminobutanamide A28;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-aminoacetamide A29;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2-amino-3-methylbutanamide A30;
(S)—N-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)-2-amino-3-(1H-imidazol-4-yl)-propanamide A31;
(S)—N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-2,6-diaminohexanamide A32;
(R)—N-((4-(6-((6-acetyl-8-cyclopentyl-5-methyl-7-oxo-7,8-dihydropyrido[2,3-d]pyrimidin-2-yl)amino)pyridin-3-yl)piperazin-1-yl)sulfonyl)pyrrolidine-2-carboxamide A33;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)acetamide A34;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)propionamide A35;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)isobutyramide A36;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)butyramide A37;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)cyclopropanecarboxamide A38;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)tetrahydrofuran-2-carboxamide A39;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)tetrahydro-2H-pyran-4-carboxamide A40;
N-((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)cyclobutanecarboxamide A41;
4-(6-((6-acetyl-8-cyclopentyl-5-methyl-7-oxo-7,8-dihydropyrido[2,3-d]-pyrimidin-2-yl)amino)pyridin-3-yl)-N-(2-hydroxyethyl)piperazine-1-sulfonamide A42;
4-(6-((6-acetyl-8-cyclopentyl-5-methyl-7-oxo-7,8-dihydropyrido[2,3-d]-pyrimidin-2-yl)amino)pyridin-3-yl)-N-(2-methoxyethyl)piperazine-1-sulfonamide A43;
((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)glycine A44;
((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)-L-valine A45;
4-(6-((6-acetyl-8-cyclopentyl-5-methyl-7-oxo-7,8-dihydropyrido[2,3-d]-pyrimidin-2-yl)amino)pyridin-3-yl)-N-methylpiperazine-1-sulfonamide A46;
4-(6-((6-acetyl-8-cyclopentyl-5-methyl-7-oxo-7,8-dihydropyrido[2,3-d]-pyrimidin-2-yl)amino)pyridin-3-yl)-N-ethylpiperazine-1-sulfonamide A47;
4-(6-((6-Acetyl-8-cyclopentyl-5-methyl-7-oxo-7,8-dihydropyrido[2,3-d]-pyrimidin-2-yl)amino)pyridin-3-yl)-N-isopropylpiperazine-1-sulfonamide A48;
4-(6-((6-acetyl-8-cyclopentyl-5-methyl-7-oxo-7,8-dihydropyrido[2,3-d]-pyrimidin-2-yl)amino)pyridin-3-yl)-N-propylpiperazine-1-sulfonamide A49;
4-(6-((6-acetyl-8-cyclopentyl-5-methyl-7-oxo-7,8-dihydropyrido[2,3-d]-pyrimidin-2-yl)amino)pyridin-3-yl)-N,N-dimethylpiperazine-1-sulfonamide A50;
6-acetyl-8-cyclopentyl-5-methyl-2-((5-(4-(morpholinosulfonyl)piperazin-1-yl)-pyridin-2-yl)amino)pyrido[2,3-d]pyrimidin-7(8H)-one A51;
6-acetyl-8-cyclopentyl-2-((5-(4-((3-hydroxyazetidin-1-yl)sulfonyl)piperazin-1-yl)pyridin-2-yl)amino)-5-methylpyrido[2,3-d]pyrimidin-7(8H)-one A52;
4-(6-((6-acetyl-8-cyclopentyl-5-methyl-7-oxo-7,8-dihydropyrido[2,3-d]-pyrimidin-2-yl)amino)pyridin-3-yl)-N,N-bis(2-hydroxyethyl)piperazine-1-sulfonamide A53;
2-hydroxyethyl (S)-((4-(6-((6-acetyl-8-cyclopentyl-5-methyl-7-oxo-7,8-dihydropyrido[2,3-d]pyrimidin-2-yl)amino)pyridin-3-yl)-3-methylpiperazin-1-yl)sulfonyl)carbamate A54;
2-((R)-2-amino-3-methylbutanamido)ethyl (((S)-4-(6-((6-acetyl-8-cyclopentyl-5-methyl-7-oxo-7,8-dihydropyrido[2,3-d]pyrimidin-2-yl)amino)pyridin-3-yl)-3-methylpiperazin-1-yl)sulfonyl)carbamate A55;
4-(6-((6-acetyl-8-cyclopentyl-5-methyl-7-oxo-7,8-dihydropyrido[2,3-d]-pyrimidin-2-yl)amino)pyridin-3-yl)-N-(isopropylcarbamoyl)piperazine-1-sulfonamide A56;
6-acetyl-2-((5-(4-((2-amino-1H-imidazol-1-yl)sulfonyl)piperazin-1-yl)pyridin-2-yl)amino)-8-cyclopentyl-5-methylpyrido[2,3-d]pyrimidin-7(8H)-one A57;
2-((5-(4-((1H-imidazol-1-yl)sulfonyl)piperazin-1-yl)pyridin-2-yl)amino)-6-acetyl-8-cyclopentyl-5-methylpyrido[2,3-d]pyrimidin-7(8H)-one A58;
(((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)oxy)methyl pivalate B1;
p-tolyl 4-(6-((6-acetyl-8-cyclopentyl-5-methyl-7-oxo-7,8-dihydropyrido[2,3-d]-pyrimidin-2-yl)amino)pyridin-3-yl)piperazine-1-sulfonate B2;
(((4-(6-((6-acetyl-8-cyclopentyl-5-methyl-7-oxo-7,8-dihydropyrido[2,3-d]-pyrimidin-2-yl)amino)pyridin-3-yl)piperazin-1-yl)sulfonyl)oxy)methyl benzoate B3;
cyclopentyl 4-(6-((6-acetyl-8-cyclopentyl-5-methyl-7-oxo-7,8-dihydropyrido[2,3-d]pyrimidin-2-yl)amino)pyridin-3-yl)piperazine-1-sulfonate B4;
6-acetyl-8-cyclopentyl-2-[[5-(4-isopropylsulfonylpiperazin-1-yl)-2-pyridyl]-amino]-5-methylpyrido[2,3-d]pyrimidin-7-one C1; or
6-acetyl-8-cyclopentyl-2-((5-(4-(ethylsulfonyl)piperazin-1-yl)pyridin-2-yl)-amino)-5-methylpyrido[2,3-d]pyrimidin-7(8H)-one C2;
or an enantiomer, a mixture of enantiomers, a diastereomer, a mixture of two or more diastereomers, a tautomer, or a mixture of two or more tautomers thereof; or a pharmaceutically acceptable salt, solvate, hydrate, or prodrug thereof.

68. A pharmaceutical composition comprising the compound of claim 1, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; and a pharmaceutically acceptable excipient.

69. (canceled)

70. A method of treating, preventing, or ameliorating one or more symptoms of a disorder, disease, or condition mediated by a CDK in a subject, comprising administering to the subject in need thereof a therapeutically effective amount of a compound of claim 1.

71. (canceled)

72. A method of preventing or ameliorating a chemotherapy-induced gastrointestinal side effect in a subject, comprising administering to the subject in need thereof a therapeutically effective amount of claim 1.

73. (canceled)

74. (canceled)

75. (canceled)

76. (canceled)

77. A method of inhibiting the activity of a CDK, comprising contacting the CDK with an effective amount of a compound of claim 1.
